(19) United States
(12) Patent Application Publication
(10) Pub. No.: US 2008/0214589 A1
(43) Pub. Date: Sep. 4, 2008

(54) Thrombin Inhibiting 2,4-Dioxo-3,4-Dihydropyrimidine Derivatives

(75) Inventors: Ingemar Nilsson, Molndal (SE); Magnus Polla, Molndal (SE)

Correspondence Address: Pepper Hamilton LLP, 400 Berwyn Park, 899 Cassatt Road, Berwyn, PA, 19312-1183, US

(73) Assignee: ASTRAZENECA AB, Sodertalje (SE)

(21) Appl. No.: 11/917,512

(22) PCT Filed: Jun. 14, 2006
(86) PCT No.: PCT/SE2006/000682

§ 371 Date: Dec. 14, 2007

(30) Foreign Application Priority Data
Jun. 17, 2005 (SE) 0501412-1

(51) Int. Cl. A61K 31/506; C07D 407/14

(52) U.S. Cl. 514/274; 544/316

(57) ABSTRACT

There is provided a compound of formula I wherein R¹ to R⁵, A, G, L and X have meanings given in the description, which compounds are useful as, or are useful as prodrugs of, competitive inhibitors of trypsin-like proteases, such as thrombin, and thus, in particular, in the treatment of conditions where inhibition of thrombin is beneficial (e.g. conditions, such as thrombo-embolisms, where inhibition of thrombin is required or desired, and/or conditions where anticoagulant therapy is indicated).

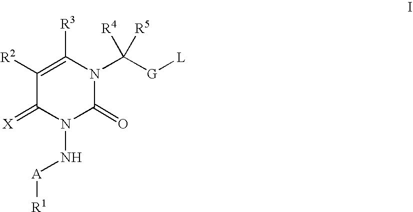

### FIELD OF THE INVENTION

This invention relates to novel pharmaceutically useful compounds, in particular compounds that are, and/or compounds that are metabolised to compounds which are, competitive inhibitors of trypsin-like serine proteases, especially thrombin, their use as medicaments, pharmaceutical compositions containing them and synthetic routes to their production.

### BACKGROUND

Blood coagulation is the key process involved in both haemostasis (i.e. the prevention of blood loss from a damaged vessel) and thrombosis (i.e. the formation of a blood clot in a blood vessel, sometimes leading to vessel obstruction).

Coagulation is the result of a complex series of enzymatic reactions. One of the ultimate steps in this series of reactions is the conversion of the proenzyme prothrombin to the active enzyme thrombin.

Thrombin is known to play a central role in coagulation. It activates platelets, leading to platelet aggregation, converts fibrinogen into fibrin monomers, which polymerise spontaneously into fibrin polymers, and activates factor XIII, which in turn crosslinks the polymers to form insoluble fibrin. Furthermore, thrombin activates factor V, factor VIII and FXI leading to a “positive feedback” generation of thrombin from prothromb in.

By inhibiting the aggregation of platelets and the formation and crosslinking of fibrin, effective inhibitors of thrombin would be expected to exhibit antithrombotic activity. In addition, antithrombotic activity would be expected to be enhanced by effective inhibition of the positive feedback mechanism. Indeed, the convincing antithrombotic effects of a thrombin inhibitor in man has recently been described by S. Schulman et al in N. Engl. J. Med. 349, 1713-1721 (2003).

### PRIOR ART

The early development of low molecular weight inhibitors of thrombin has been described by Claesson in Blood Coagul. Fibrinol. 5, 411 (1994).

Blombäck et al (in J. Clin. Lab. Invest. 24, suppl. 107, 59 (1969)) reported thrombin inhibitors based on the amino acid sequence situated around the cleavage site for the fibrinogen Aa chain. Of the amino acid sequences discussed, these authors suggested the tripeptide sequence Phe-Val-Arg (P9-P2-P1, hereinafter referred to as the P3-P2-P1 sequence) would be the most effective inhibitor.

Thrombin inhibitors based on peptidyl derivatives, having cyclic or acyclic basic groups at the P1-position (e.g. groups containing amino, amidino or guanidino functions), are disclosed in, for example, International Patent Application numbers WO 93/11152, WO 93/18060, WO 94/29336, WO 95/23609, WO 95/35309, WO 96/03374, WO 96/25426, WO 96/31504, WO 96/32110, WO 97/02284, WO 97/23499, WO 97/46577, WO 97/49404, WO 98/06740, WO 98/57932, WO 99/29664, WO 00/35869, WO 00/42059, WO 01/87879, WO 02/14270, WO 02/44145 and WO 03/018551, European Patent Application numbers 185 390, 468 231, 526 877, 542 525, 559 046 and 641 779, 648 780, 669 317 and U.S. Pat. No. 4,346,078.

Inhibitors of serine proteases (e.g. thrombin) based on electrophilic ketones in the PI-position are also known, such as the compounds disclosed in European Patent Application numbers 195 212, 362 002, 364 344 and 530 167.

Inhibitors of trypsin-like serine proteases based on C-terminal boronic acid derivatives of arginine (and isothiouronium analogues thereof) are known from European Patent Application number 293 881.

Achiral thrombin inhibitors having, at the P2-position of the molecule, a phenyl group, and a cyclic or acyclic basic group at the P3-position, are disclosed in International Patent Application numbers WO 94/20467, WO 96/06832, WO 96/06849, WO 97/11693, WO 97/24135, WO 98/01422 and WO 01/68605, as well as in Bioorg. Med. Chem. Lett. 7, 1283 (1997).

International Patent Application numbers WO 99/26920 and WO 01/79155 disclose thrombin inhibitors having groups at the P2-position based, respectively, upon 2-aminophenols and 1,4-benzoquinones. Similar, phenol-based compounds are also disclosed in International Patent Application numbers WO 01/68605 and WO 02/28825.

Further known inhibitors of thrombin and other trypsin-like serine proteases are based (at the P2-position of the molecule) on the 3-amino-2-pyridone structural unit. For example, compounds based upon 3-amino-2-pyridone, 3-amino-2-pyrazinone, 5-amino-6-pyrimidone, 5-amino-2,6-pyrimidione and 5-amino-1,3,4-triazin-6-one are disclosed in International Patent Application numbers WO 96/18644, WO 97/01338, WO 97/30708, WO 98/16547, WO 99/26926, WO 00/73302, WO 00/75134, WO 01/38323, WO 01/04117, WO 01/70229, WO 01/79262, WO 02/057225, WO 02/064140 and WO 03/29224, U.S. Pat. Nos. 5,668,289 and 5,792,779, as well as in Bioorg. Med. Chem. Lett. 8, 817 (1998) and J. Med. Chem. 41, 4466 (1998).

Thrombin inhibitors based upon the pyridin-2-amine 1-oxide structural unit are disclosed in International Patent Application number WO 02/042272 and in US patent application number US 2003/158218.

Thrombin inhibitors based upon 2-oxo-3-amino-substituted saturated azaheterocycles are disclosed in International Patent Application number WO 95/35313. More recently, thrombin inhibitors have been disclosed that are based upon 4-amino-3-morpholinone (see J. Med. Chem. 46, 1165 (2003)). Further, compounds based upon the structural unit 1-amino-2-pyridone, as well as its di- and tetra-hydrogenated analogues, are described in unpublished international patent application numbers PCT/SE2004/001878 and PCT/SE2005/000124.

None of the above-mentioned documents disclose or suggest compounds based (at the P2-position) on the 1-amino-2,6-pyrimidione structural unit.

Moreover, there remains a need for effective inhibitors of trypsin-like serine proteases, such as thrombin. There is also a need for compounds that have a favourable pharmacokinetic profile. Such compounds would be expected to be useful as anticoagulants and therefore in the therapeutic treatment of thrombosis and related disorders.

### DISCLOSURE OF THE INVENTION

According to the invention there is provided a compound of formula I

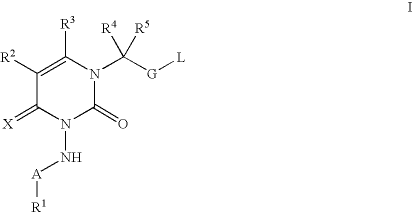

wherein
X represents O or S;
A represents C(O), S(O)₂, C(O)O (in which latter group the 0 moiety is attached to R¹), C(O)NH, S(O)₂NH (in which latter two groups the NH moiety is attached to R¹), a direct bond or C₁₋₆ alkylene (which latter group is optionally substituted, at the C-atom to which the NH moiety is attached, by C(O)OR^{A} or C(O)N(H)R^{A});
R^{A} represents H or C₁₋₄ alkyl;
R¹ represents
(a) C₁₋₁₀ alkyl, C₂₋₁₀ alkenyl, C₂₋₁₀ alkynyl (which latter three groups are optionally substituted by one or more substituents selected from halo, CN, C₃₋₁₀ cycloalkyl (optionally substituted by one or more substituents selected from halo, OH, ═O, C₁₋₆ alkyl, C₁₋₆ alkoxy and aryl), OR^{6a}, S(O)ₙR^{6b}, S(O)₂N(R^{6c})(R^{6d}), N(R^{6e})S(O)₂R^{6f}, N(R^{6g})(R^{6h}), B¹—C(O)—B²—R⁶ⁱ, aryl and Het¹),
(b) C₃₋₁₀ cycloalkyl or C₄₋₁₀ cycloalkenyl, which latter two groups are optionally substituted by one or more substituents selected from halo, ═O, CN, C₁₋₁₀ alkyl, C₃₋₁₀ cycloalkyl (optionally substituted by one or more substituents selected from halo, OH, ═O, C₁₋₆ alkyl, C₁₋₆ alkoxy and aryl), OR^{6a}, S(O)ₙR^{6b}, S(O)₂N(R^{6c})(R^{6d}), N(R^{6e})S(O)₂R^{f}, N(R^{6g})(R^{6h}), B³—C(O)—B⁴—R⁶ⁱ, aryl and Het²,
(c) aryl, or
(d) Het³;
   R^{6a} to R⁶ⁱ independently represent, at each occurrence,
(a) IL
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, aryl and Het⁴),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, aryl and Her),
(d) aryl or
(e) Het⁶,
   provided that R^{6b} does not represent H when n is 1 or 2;
   R² represents H or halo;
   R³ represents
(a) H,
(b) halo,
(c) CN,
(d) C₁₋₆ alkyl, C₂₋₆ alkenyl, C₂₋₆ alkynyl, C₁₋₆ alkoxy (which latter four groups are optionally substituted by one or more substituents selected from halo, OH, CN, C₁₋₄ alkoxy, C(O)OH, C(O)O—C₁₋₄ alkyl and OC(O)—C₁₋₄ alkyl) or
(e) together with R⁴, R³ represents C₂₋₃ n-alkylene, T¹-(C₁₋₂ n-alkylene) or (C₁₋₂ n-alkylene)-T¹, which latter three groups are optionally substituted by halo, or
(f) together with R⁴ and R⁵, R³ represents T²-[C(H)═], wherein T² is bonded to the C-atom to which the group R³ is attached;
   R⁴ and R⁵ independently represent H, F or methyl (which latter group is optionally substituted by one or more F atoms), or
(a) together with R³, R⁴ represents C₂₋₃ n-alkylene, T¹-(C₁₋₂ n-allylene) or (C₁₋₂ n-alkylene)-T₁, which latter three groups are optionally substituted by halo, or
(b) together with R³, R⁴ and R⁵ represent T²-[C(H)═], wherein T² is bonded to the C-atom to which the group R³ is attached;
   T¹ and T² independently represent O, S, or NR⁷;
   R⁷ represents H or C₁₋₄ alkyl;
   G represents
   (a) —C(R^{7a})(R^{7b})N(R^{8a})—[CH(C(O)R⁹)]₀₋₁—C₀₋₃ alkylene-(Q¹)ₐ-,
   (b) —C(R^{7a})(R^{7b})(O)N(R^{8b})—C₂₋₃ alkenylene-(Q¹)ₐ-,
   (c)

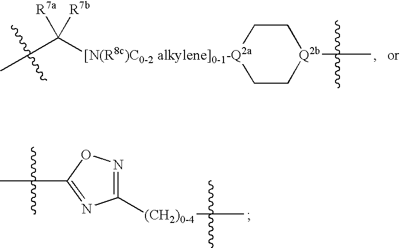

R^{7a} and R^{7b} independently represent H or methyl, or R^{7a} and R^{7b} together represent ═O;
R⁹ represents H or a 5- to 10-membered aromatic heterocyclic group comprising one or two rings and containing, as heteroatom(s), one sulfur or oxygen atom and/or one or more nitrogen atoms, which heterocyclic group is optionally substituted by one or more substituents selected from halo and C₁₋₆ alkyl;
Q¹ represents O, NR^{10a}, [N(H)]₀₋₁C(O)—C₀₋₂ alkylene, C(O)NHNHC(O), or —N═C(R^{10a})—;
a represents 0 or 1;
Q^{2a} represents

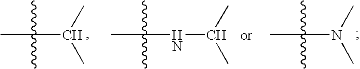

Q^{2b} represents

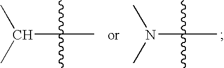

L represents
(a) C₀₋₆ alkylene-R^{a},
(b) C₀₋₂ alkylene-CH═CH—C₀₋₂ alkylene-R^{a},
(c) C₀₋₂ alkylene-C═C—C₀₋₂ alkylene —R^{a},

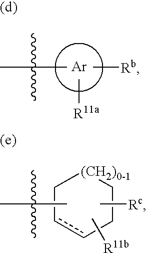

wherein the dashed line represents an optional double bond, or

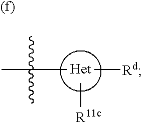

Ar represents phenyl or naphthyl;
Het represents a 5- to 10-membered heterocyclic group comprising one or two rings and containing, as heteroatom(s), one sulfur or oxygen atom and/or one or more nitrogen atoms;
R^{11a} represents H or one or more substituents selected from halo, OH, CN, C₁₋₆ alkyl, C₁₋₆ alkoxy (which latter two groups are optionally substituted by one or more substituents selected from halo, OH, C₁₋₄ alkoxy, C(O)OR^{12a} and C(O)N(R¹²R^{12c}) and S(O)₀₋₂R^{12d};
R^{11b} and R^{11c} independently represent H or one or more substituents selected from halo, OH, CN, C₁₋₆ alkyl, C₁₋₆ alkoxy (which latter two groups are optionally substituted by one or more substituents selected from halo, OH, C₁₋₄ alkoxy, C(O)OR^{12a} and C(O)N(R^{12b})R^{12c}), S(O)₀₋₂R^{12d}, ═O, ═NH, ═NOH and ═N—CN;
R^{12a} to R^{12c} independently represent H, C₁₋₆ alkyl or C₃₋₇ cycloalkyl (which latter two groups are optionally substituted by one OH or N(R^{12e})R^{12f} group or by one or more halo atoms);
R^{12d} represents, independently at each occurrence, C₁₋₆ alkyl optionally substituted by one OH or N(R^{12e})R^{12f} group or by one or more halo atoms;
R^{12e} and R^{12f} represent, independently at each occurrence, H or C₁₋₄ alkyl optionally substituted by one or more halo atoms;
R^{a} to R^{d} independently represent

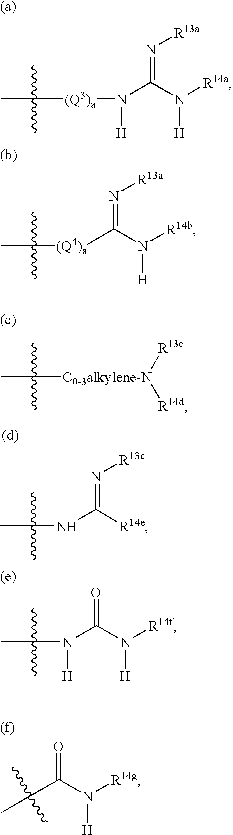

### (g) Het^{x}

or R^{b} to R^{d} may also represent H;
Q³ represents O, N(R^{10c}), S(O)₂, S(O)₂NH, C(O) or —CH═N—;
Q⁴ represents O, S or CH₂;
a represents 0 or 1;
Het^{x} represents a 5- or 6-membered heterocyclic group containing one to four heteroatoms selected from oxygen, nitrogen and/or sulfur, which heterocyclic group may be substituted by one or more substituents selected from halo, ═O, C₁₋₆ alkyl and C₁₋₆ alkoxy (which latter two groups are optionally substituted by one or more halo atoms);
R^{13a} to R^{13c} independently represent
(a) H,
(b) CN,
(c) NH₂,
(d) OR¹⁵ or
(e) C(O)OR¹⁶;
R¹⁵ represents
(a) H,
(b) C₁₋₁₀ alkyl, C₃₋₁₀ alkenyl, C₃₋₁₀ alkynyl,
(c) C₃₋₁₀ cycloalkyl, C₄₋₁₀ cycloalkenyl, which latter two groups are optionally substituted by one or more substituents selected from halo and C₁₋₆ alkyl, or
(d) C₁₋₃ alkyl, which latter group is optionally interrupted by oxygen and is substituted by aryl or —O-aryl;
   R¹⁶ represents
(a) C₁₋₁₀ alky, C₃₋₁₀ alkenyl, C₃₋₁₀ alkynyl, which latter three groups are optionally interrupted by one or more oxygen atoms, or
(b) C₃₋₁₀ cycloalkyl, C₄₋₁₀ cycloalkenyl, which latter two groups are optionally substituted by one or more substituents selected from halo and C₁₋₆ alkyl, or
(c) C₁₋₃ alkyl, which latter group is optionally interrupted by oxygen and is substituted by aryl or —O-aryl;
   R^{8a} to R^{8c}, R^{10a} to R^{10c} and R^{14a} to R^{14g} independently represent
(a) H or
(b) C₁₋₄ alkyl (which latter group is optionally substituted by one or more substituents selected from halo and OH),
   or R^{14a} and R^{14b} independently represent C(O)O—C₁₋₆ alkyl (the alkyl part of which latter group is optionally substituted by aryl and/or one or more halo atoms),
   or R^{14c} represents
(a) C₁₋₄ alkyl substituted by C₃₋₇ cycloalkyl or aryl,
(b) C₃₋₇ cycloalkyl,
(c) C(O)O—C₁₋₆ alkyl (the alkyl part of which latter group is optionally substituted by aryl and/or one or more halo atoms),
(d) C(O)C₁₋₆ alkyl,
(e) C(O)N(H)—C₁₋₆ alkyl (the alkyl part of which latter group is optionally substituted by aryl and/or one or more halo atoms) or
(f) S(O)₂—C₁₋₆ alkyl (the alkyl part of which latter group is optionally substituted by aryl and/or one or more halo atoms),
   or R^{14c} and R^{14d} together represent C₃₋₆ n-alkylene optionally interrupted by O, S, N(H) or N(C₁₋₄ alkyl) and/or substituted by one or more C₁₋₄ alkyl groups;
   each aryl independently represents a C₆₋₁₀ carbocyclic aromatic group, which group may comprise either one or two rings and may be substituted by one or more substituents selected from
(a) halo,
(b) CN,
(c) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, C(O)OH, C(O)O—C₁₋₆ alkyl, phenyl (which latter group is optionally substituted by halo) and Het⁷),
(d) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het⁸),
(e) OR^{17a},
(f) S(O)ₚR^{17b},
(g) S(O)₂N(R^{17c})(R^{17d}),
(h) N(R^{17e})S(O)₂R^{17f},
(i) N(R^{17g})(R^{17h}),
(j) B⁵—C(O)—B⁶—R¹⁷ⁱ,
(k) phenyl (which latter group is optionally substituted by halo),
(l) Het⁹ and
(m) Si(R^{18a})(R^{18b})(R^{18c});
   R^{17a} to R¹⁷ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, phenyl (which latter group is optionally substituted by halo) and Het¹⁰),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het¹¹),
(d) phenyl (which latter group is optionally substituted by halo) or
(e) Het¹²,
   provided that R^{17b} does not represent H when p is 1 or 2;
   Het¹ to Het¹² independently represent 4- to 14-membered heterocyclic groups containing one or more heteroatoms selected from oxygen, nitrogen and/or sulfur, which heterocyclic groups may comprise one, two or three rings and may be substituted by one or more substituents selected from
(a) halo,
(b) CN,
(c) C₁₋₁₀ alky, C₂₋₁₀ alkenyl, C₂₋₁₀ alkynyl (which latter four groups are optionally substituted by one or more substituents selected from halo, OH, C₁₋₆ alkoxy, C(O)OH, C(O)O—C₁₋₆ alkyl, phenyl (which latter group is optionally substituted by halo) and Het^{a}),
(d) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het^{b}),
(e) ═O,
(f) OR^{19a},
(g) S(O)_{q}R^{19b},
(h) S(O)₂N(R^{19c})(R^{19d}),
(i) N(R^{19e})S(O)₂R^{19f},
(j) N(R^{19g})(R^{19h}),
(k) B⁷—C(O)—B⁸—R¹⁹ⁱ,
(l) phenyl (which latter group is optionally substituted by halo),
(m) Het^{c} and
(n) Si(R^{20a})(R^{20b})(R^{20c});
   R^{19a} to R¹⁹ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, phenyl (which latter group is optionally substituted by halo) and Het^{d}),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het^{e}),
(d) phenyl (which latter group is optionally substituted by halo) or
(e) Het^{f},
   provided that R^{19b} does not represent H when q is 1 or 2;
   Het^{a} to Het^{f} independently represent 5- or 6-membered heterocyclic groups containing one to four heteroatoms selected from oxygen, nitrogen and/or sulfur, which heterocyclic groups may be substituted by one or more substituents selected from halo, ═O and C₁₋₆ alkyl;
   B¹ to B⁸ independently represent a direct bond, O, S, NH or N—C₁₋₄ alkyl;
   n, p and q independently represent 0, 1 or 2;
   R^{18a}, R^{18b}, R^{18c}, R^{20a}, R^{20b} and R^{20c} independently represent C₁₋₆ alkyl or phenyl (which latter group is optionally substituted by halo or C₁₋₄ alkyl);
   unless otherwise specified
(i) alkyl, alkenyl, alkynyl, cycloalkyl, cycloalkenyl, alkylene and alkenylene groups, as well as the alkyl part of alkoxy groups, may be substituted by one or more halo atoms, and
(ii) cycloalkyl and cycloalkenyl groups may comprise one or two rings and may additionally be ring-fused to one or two phenyl groups;
   or a pharmaceutically-acceptable derivative thereof,
   which compounds are referred to hereinafter as “the compounds of the invention”.

The term “pharmaceutically-acceptable derivatives” includes pharmaceutically-acceptable salts (e.g. acid addition salts).

For the avoidance of doubt, the definitions of the terms aryl, alkyl, alkenyl, alkynyl, cycloalkyl, cycloalkenyl, alkylene, alkenylene and alkoxy groups provided above apply, unless otherwise stated, at each usage of such terms herein. The term “halo”, when used herein, includes fluoro, chloro, bromo and iodo.

Heterocyclic (Het, Het¹ to Het¹², Het^{a} to Het^{f} and Het^{x}) groups may be fully saturated, partly unsaturated, wholly aromatic or partly aromatic in character. Values of heterocyclic (Het, Het¹ to Het¹², Het^{a} to Het^{f} and Het^{x}) groups that may be mentioned include 1-azabicyclo[2.2.2]octanyl, benzimidazolyl, benzo[c]-isoxazolidinyl, benzisoxazolyl, benzodioxanyl, benzodioxepanyl, benzodioxolyl, benzofuranyl, benzofurazanyl, benzomorpholinyl, 2,1,3-benzoxadiazolyl, benzoxazolidinyl, benzoxazolyl, benzopyrazolyl, benzo[e]pyrimidine, 2,1,3-benzothiadiazolyl, benzothiazolyl, benzothienyl, benzotriazolyl, chromanyl, chromenyl, cinnolinyl, 2,3-dihydrobenzimidazolyl, 2,3-dihydrobenzo[b]furanyl, 1,3-dihydrobenzo[c]furanyl, 1,3-dihydro-2,1-benzisoxazolyl 2,3-dihydro-pyrrolo[2,3-b]pyridinyl, dioxanyl, furanyl, hexahydropyrimidinyl, hydantoinyl, imidazolyl, imidazo[1,2-a]pyridinyl, imidazo[2,3-b]thiazolyl, indolyl, isoquinolinyl, isoxazolidinyl, isoxazolyl, maleimido, morpholinyl, naphtho[1,2-b]furanyl, oxadiazolyl, 1,2- or 1,3-oxazinanyl, oxazolyl, phthalazinyl, piperazinyl, piperidinyl, purinyl, pyranyl, pyrazinyl, pyrazolyl, pyridinyl, pyridonyl, pyrimidinyl, pyrrolidinonyl, pyrrolidinyl, pyrrolinyl, pyrrolo[2,3-b]pyridinyl, pyrrolo[5,1-b]pyridinyl, pyrrolo[2,3-c]pyridinyl, pyrrolyl, quinazolinyl, quinolinyl, sulfolanyl, 3-sulfolenyl, 4,5,6,7-tetrahydrobenzimidazolyl, 4,5,6,7-tetrahydrobenzopyrazolyl, 5,6,7,8-tetrahydrobenzo[e]pyrimidine, tetra-hydrofuranyl, tetrahydropyranyl, 3,4,5,6-tetrahydropyridinyl, 1,2,3,4-tetrahydro-pyrimidinyl, 3,4,5,6-tetrahydropyrimidinyl, thiadiazolyl, thiazolidinyl, thiazolyl, thienyl, thieno[5,1-c]pyridinyl, thiochromanyl, triazolyl, 1,3,4-triazolo[2,3-b]pyrimidinyl, xanthenyl and the like.

Values of Het that may be mentioned include 1-azabicyclo[2.2.2]octanyl, benzimidazolyl, benzo[c]isoxazolidinyl, benzisoxazolyl, benzo[b]furanyl, benzo-pyrazolyl, benzo[e]pyrimidine, benzothiazolyl, benzo[b]thienyl, benzotriazolyl, 2-oxo-2,3-dihydrobenzimidazolyl, 1,3-dihydro-2,1-benzisoxazolyl, 2,3-dihydro-pyrrolo[2,3-b]pyridinyl, furanyl, 2-imino-hexahydropyrimidinyl, imidazolyl, imidazo[1,2-a]pyridinyl, indolyl, isoquinolinyl, isoxazolidinyl, isoxazolyl, 1,2,4-oxadiazolyl, 1,3,4-oxadiazolyl, 1,2-oxazinanyl, 2-imino-1,3-oxazinanyl, piperazinyl, piperidinyl, 2-oxo-piperidinyl, pyrazinyl, pyridinyl, pyrimidinyl, 2-imino-pyrrolidinyl, 3-pyrrolinyl, pyrrolo[2,3-b]pyridinyl, pyrrolo[5,1-b]pyridinyl, pyrrolo[2,3-c]pyridinyl, pyrrolyl, quinolinyl, 4,5,6,7-tetrahydrobenz-imidazolyl, 4,5,6,7-tetrahydrobenzopyrazolyl, 5,6,7,8-tetrahydrobenzo[e]-pyrimidine, 3,4,5,6-tetrahydro-pyridinyl, 3,4,5,6-tetrahydropyrimidinyl, 2-imino-thiazolidinyl, thiazolyl, thienyl and thieno[5,1-c]pyridinyl.

Values of Het¹ that may be mentioned include benzodioxolyl, benzo[b]furanyl, 2,3-dihydrobenzo[b]furanyl, pyridinyl, pyrimidinyl and thienyl.

Values of Het³ that may be mentioned include benzodioxanyl, benzo[b]dioxepanyl, benzodioxolyl, benzomorpholinyl, 2,1,3-benzoxadiazolyl, 2-oxo-benzoxazolidinyl, benzopyrazolyl, 2,1,3-benzothiadiazolyl, benzo[b]-thienyl, 2-oxo-chromenyl, 2,3-dihydrobenzo[b]furanyl, 1-oxo-1,3-dihydro-benzo[c]furanyl, furanyl, imidazolyl, imidazo[2,3-b]thiazolyl, isoquinolinyl, isoxazolyl, naphtho[1,2-b]furanyl, pyrazinyl, pyrazolyl, pyridinyl, pyridonyl, pyrrolyl, quinolinyl, sulfolanyl, 3-sulfolenyl, 2,4-dioxo-1,2,3,4-tetrahydropyrimidinyl, thiazolyl, thienyl, 1,3,4-triazolo[2,3-b]pyrimidinyl and xanthenyl.

Values of Het⁹ that may be mentioned include morpholinyl, 1,3,4-oxadiazolyl, oxazolyl and pyrazolyl.

Values of Het¹⁰ that may be mentioned include isoxazolyl, oxazolyl and thiazolyl.

Values of Het^{c} that may be mentioned include isoxazolyl, morpholinyl, oxazolyl, pyridinyl, thienyl and triazolyl (e.g. 1,3,4-triazolyl).

Values of Het^{x} that may be mentioned include dihydrooxadiazolyl (e.g. 4,5-dihydro-1,2,4-oxadiazol-3-yl), oxadiazolyl (e.g. 1,2,4-oxadiazol-3-yl), tetrazolyl (e.g. triazol-1-yl) and triazolyl (e.g. 1,2,4-triazol-1-yl).

Substituents on heterocyclic (Het, Het¹ to Het¹², Het^{a} to Het^{f} and Het^{x}) groups may, where appropriate, be located on any atom in the ring system including a heteroatom. The point of attachment of heterocyclic (Het, Het¹ to Het¹², Het^{a} to Het^{f} and Het^{x}) groups may be via any atom in the ring system including (where appropriate) a heteroatom, or an atom on any fused carbocyclic ring that may be present as part of the ring system.

For the avoidance of doubt, cycloalkyl and cycloalkenyl groups may be monocyclic or, where the number of C-atoms allows, be bi- or tricyclic (although monocyclic cycloalkyl and cycloalkenyl are particular embodiments that may be mentioned). Further, when a cycloalkyl or cycloalkenyl group is fused to two phenyl groups, the phenyl groups may also be fused to each other (to form a fused tricyclic ring system).

Compounds of formula I may exhibit tautomerism. All tautomeric forms and mixtures thereof are included within the scope of the invention.

Compounds of formula I may also contain one or more asymmetric carbon atoms and may therefore exhibit optical and/or diastereoisomerism. Diastereoisomers may be separated using conventional techniques, e.g chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation, or by derivatisation, for example with a homochiral acid followed by separation of the diastereomeric esters by conventional means (e.g. HPLC, chromatography over silica). All stereoisomers are included within the scope of the invention.

Abbreviations are listed at the end of this specification. The wavy lines on the bonds in structural fragments signify the bond positions of those fragments. Compounds of formula I that may be mentioned include those in which R^{7a} and R^{7b} together represent ═O. In this respect, particular values of G that may be mentioned include:

(a) —C(O)N(R^{8a})—C₀₋₃ alkylene-;
(b) —C(O)N(R^{8a})—CH(C(O)R⁹)—C₀₋₃ alkylene-;
(c) —C(O)N(R^{8a})—C₁₋₃ alkylene-Q¹-;
(d) —C(O)N(R^{8b})—C₂₋₃ alkenylene-;

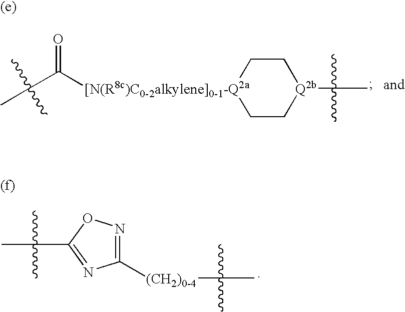

When G represents —C(O)N(R^{8a})—C₀₋₃ alkylene-Q¹-, particular values of L that may be mentioned include:

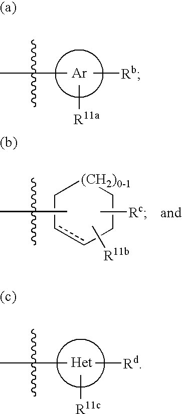

When G represents —C(O)N(R^{8b})—C₂₋₃ alkenylene-,

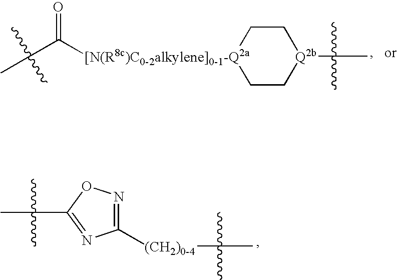

particular values of L that may be mentioned include:

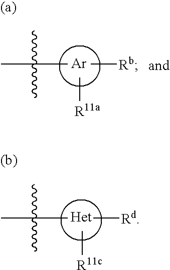

Particular values that may be mentioned in relation to compounds of formula I include those in which:
(1) A represents C(O), S(O)₂, C(O)NH (in which latter group the NH moiety is attached to R¹) or C₁₋₄ alkylene;
(2) R¹ represents
   (a) C₁₋₆ alkyl, C₂₋₆ alkenyl, C₂₋₆ alkynyl (which latter three groups are optionally substituted by one or more substituents selected from halo, CN, C₃₋₈ cycloalkyl (optionally substituted by one or more substituents selected from halo, OH, ═O, C₁₋₆ alkyl, C₁₋₆ alkoxy and aryl), OR^{6a}, SR^{6b}, S(O)₂R^{6b}, S(O)₂N(H)R^{6c}, N(H)S(O)₂R^{6f}, N(R^{6g})(R^{6h}), C(O)R⁶ⁱ, OC(O)R⁶ⁱ, C(O)OR⁶ⁱ, N(H)C(O)R⁶ⁱ, C(O)N(H)R⁶ⁱ, aryl and Het¹),
   (b) C₃₋₈ cycloalkyl or C₄₋₈ cycloalkenyl, which latter two groups are optionally fused to one or two phenyl groups and are optionally substituted by one or more substituents selected from halo, ═O, C₁₋₆ alkyl, C₄₋₆ cycloalkyl (optionally substituted by one or more substituents selected from halo, C₁₋₄ alkyl, C₁₋₄ alkoxy and phenyl), OR^{6a}, SR^{6b}, S(O)₂R^{6b}, S(O)N(H)R^{6c}, N(H)S(O)₂R^{6f}, N(R^{6g})(R^{6h}), OC(O)R⁶ⁱ, C(O)OR⁶ⁱ, N(H)C(O)R⁶ⁱ, C(O)N(H)R⁶ⁱ, aryl and Het²,
   (c) aryl, or
   (d) Het³;
(3) R^{6a} to R⁶ⁱ independently represent, at each occurrence,
   (a) H
   (b) C₁₋₆ alkyl, C₂₋₆ alkenyl, C₂₋₆ alkynyl (which latter three groups are optionally substituted by one or more substituents selected from halo, OH, C₁₋₄ alkoxy, aryl and Het⁴),
   (c) C₄₋₆ cycloalkyl, C₄₋₆ cycloalkenyl (which latter two groups are optionally substituted by one or more substituents selected from halo, ═O and C₁₋₄ alkyl),
   (d) aryl or
   (e) Het⁶,
   provided that R^{6b} does not represent H when n is 1 or 2;
(4) R² represents H, F or Cl;
(5) R³ represents H; halo, CN, C₁₋₄ alkoxy (which latter group is substituted by one or more F atoms) or C₁₋₄ alkyl (which latter group is optionally substituted by one or more substituents selected from halo (e.g. F), OH or methoxy);
(6) R⁴ and R⁵ independently represent H or F;
(7) the group G-L takes any of the following definitions
   (a) C(O)N(R^{8a})—C₀₋₆ alkylene-R^{a},
   (b) C(O)N(R^{8a})—CH(C(O)R⁹)—C₀₋₅ alkylene-R^{a},
   (c) C(O)N(R^{8a})—C₀₋₃ alkylene-CH═CH—C₀₋₂ alkylene-R^{a},
   (d) C(O)N(R^{8a})—C₀₋₃ alkylene-C═C—C₀₋₂ alkylene-R^{a},

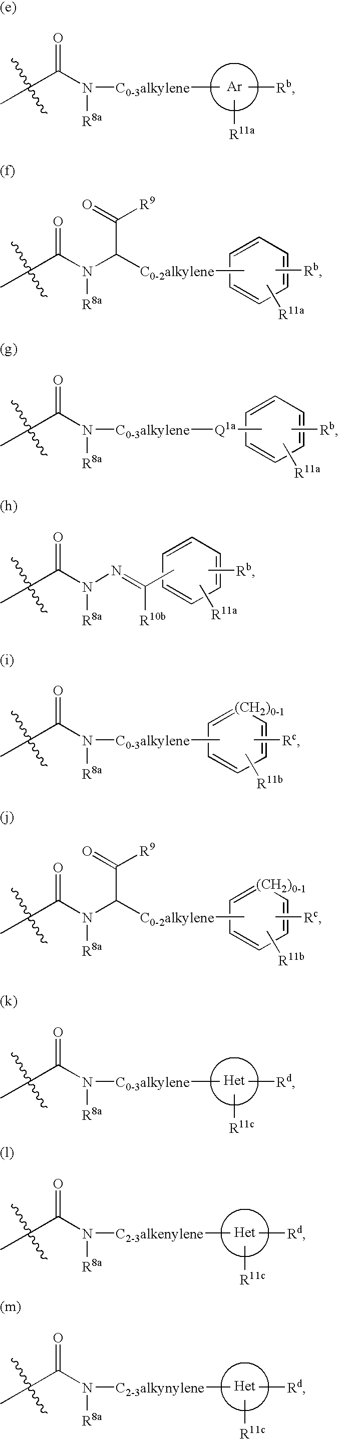

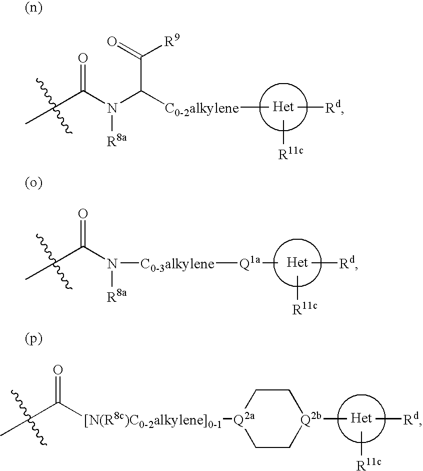

wherein Q^{1a} represents O, NR^{10a} or [N(H)]₀₋₁ C(O)—C₀₋₂ alkylene;
(8) R⁹ represents a 5- to 10-membered aromatic heterocyclic group comprising one or two rings and containing, as heteroatom(s), one sulfur or oxygen atom and/or one to three nitrogen atoms, which heterocyclic group is optionally substituted by one or more substituents selected from halo and C₁₋₄ alkyl;
(9) Het represents a 5- or 6-membered monocyclic, or a 8-, 9- or 10-membered bicyclic heterocyclic group containing, as heteroatom(s), one sulfur or oxygen atom and/or one to four nitrogen atoms;
(10) R^{11a} represents H or one to three substituents selected from halo, OH, CN, C₁₋₄ alkyl and C₁₋₄ alkoxy (which latter two groups are optionally substituted by one or more substituents selected from OH, halo, C(O)OR^{12a} and C(O)N(R^{12b})R^{12c});
(11) R^{11b} represents H or one to three substituents selected from halo, OH, C₁₋₄ alkyl, C₁₋₄ alkoxy and ═O;
(12) R^{11c} represents H or one to three substituents selected from halo, OH, CN, C₁₋₄ alkyl, C₁₋₄ alkoxy (which latter two groups are optionally substituted by one or more substituents selected from halo, OH and C₁₋₂ alkoxy), ═O, ═NH, ═NOH and —N—CN;
(13) R^{12a} to R^{12c} independently represent H, C₁₋₄ alkyl (optionally substituted by one N(R^{12e})R^{12f} group) or C₃₋₆ cycloalkyl;
(14) R^{a} represents

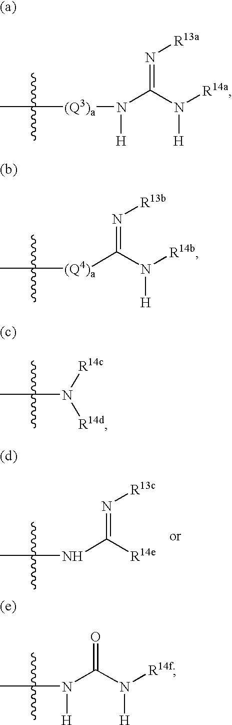

(15) R^{b} represents
   (a) H,

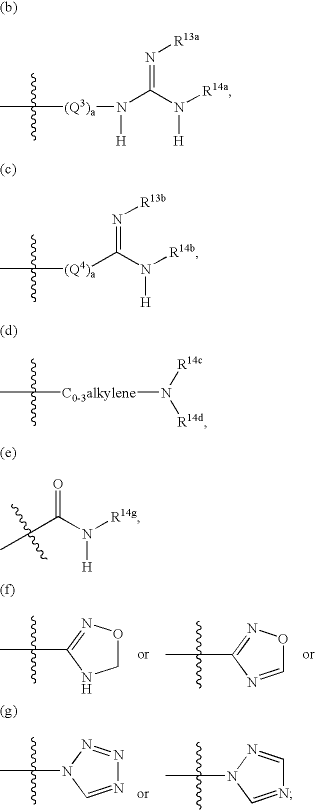

(16) R^{c} and R^{d} independently represent

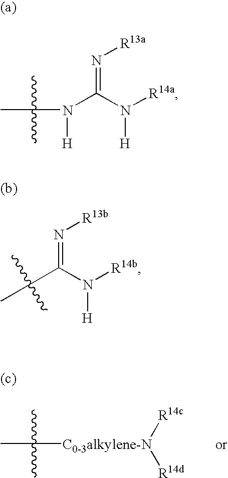

(d) R^{d} may also represent H;
(17) Q³ represents O, S(O)₂, S(O)₂NH, C(O) or —CH═N—;
(18) Q⁴ represents O or S;
(19) R¹⁵ represents H, C₁₋₆ alkyl, C₃₋₆ alkenyl (which latter two groups are optionally interrupted by an oxygen atom), C₃₋₆ cycloalkyl or C₁₋₂ alkyl
   (which latter group is substituted by aryl);
(20) R¹⁶ represents C₁₋₆ alkyl, C₃₋₆ alkenyl, C₃₋₆ cycloalkyl or C₁₋₂ allyl substituted by aryl;
(21) R^{8a} to R^{8c} represent H or methyl;
(22) R^{10a} to R^{10c} independently represent H or C₁₋₃ alkyl (which latter group is optionally substituted by OH or one or more halo atoms);
(23) R^{14a} represents C₁₋₂ alkyl, C(O)O—C₁₋₅ alkyl (the alkyl part of which latter group is optionally substituted by phenyl) or H;
(24) R^{14b} to R^{14g} independently represents H or C₁₋₂ alkyl (which latter group is optionally substituted by one or more halo atoms), or R^{14c} represents C₄₋₆ cycloalkyl or C(O)O—C₁₋₅ alkyl (the alkyl part of which latter group is optionally substituted by phenyl) or R^{4c} and R^{14d} together represent C₄₋₅ n-alkylene optionally interrupted by O;
(25) each aryl independently represents phenyl or naphthyl, each of which groups may be substituted by one or more substituents selected from
   (a) halo,
   (b) CN,
   (c) C₁₋₈ alkyl, C₂₋₄ alkenyl, C₂₋₄ alkynyl (which latter three groups are optionally substituted by one or more substituents selected from halo, OH, C₁₋₂ alkoxy, C(O)OH, C(O)O—C₁₋₂ alkyl and phenyl),
   (d) C₃₋₆ cycloalkyl optionally substituted by one or more substituents selected from halo, ═O and C₁₋₄ alky,
   (e) OR^{17a},
   (f) SR^{17b}, S(O)₂R^{17b},
   (g) S(O)₂N(H)R^{17c},
   (h) N(H)S(O)₂R^{17f},
   (i) N(H)R^{17g},
   (j) C(O)R¹⁷ⁱ, C(O)OR¹⁷ⁱ, OC(O)R¹⁷ⁱ, C(O)N(H)R¹⁷ⁱ, N(H)C(O)R¹⁷ⁱ, N(H)C(O)OR¹⁷ⁱ,
   (k) phenyl (which latter group is optionally substituted by one or more halo atoms),
   (l) Het⁹ and
   (m) Si(CH₃)₃;
(26) R^{17a} to R¹⁷ⁱ independently represent, at each occurrence,
   (a) H,
   (b) C₁₋₅ alkyl optionally substituted by one or more substituents selected from halo, OH, C₁₋₂ alkoxy, phenyl (which latter group is optionally substituted by one or more halo atoms) and Het¹⁰,
   (c) C₃₋₆ cycloalkyl optionally substituted by one or more substituents selected from halo, ═O and C₁₋₄ alkyl,
   (d) phenyl optionally substituted by one or more halo atoms or
   (e) Het¹²,
   provided that R^{17b} does not represent H;
(27) Het¹ to Het¹² independently represent 5- to 13-membered heterocyclic groups containing one to four heteroatoms selected from oxygen, nitrogen and/or sulfur, which heterocyclic groups may comprise one, two or three rings and may be substituted by one or more substituents selected from
   (a) halo,
   (b) CN,
   (c) C₁₋₈ alkyl, C₂₋₄ alkenyl, C₂₋₄ alkynyl (which latter three groups are optionally substituted by one or more substituents selected from halo, OH and C₁₋₂ alkoxy),
   (d) C₃₋₆ cycloalkyl optionally substituted by one or more substituents selected from halo, ═O and C₁₋₄ alkyl,
   (e) ═O,
   (f) OR^{19a},
   (g) S(O)₂R^{19b},
   (h) S(O)₂N(H)R^{19c},
   (i) N(H)S(O)₂R^{19f},
   (j) N(H)R^{19g},
   (j) C(O)R¹⁹ⁱ, C(O)OR¹⁹ⁱ, C(O)N(H)R¹⁹ⁱ, N(H)C(O)R¹⁹ⁱ, N(H)C(O)OR¹⁹ⁱ,
   (l) phenyl (which latter group is optionally substituted by halo) and
   (m) Het^{c};
(28) R^{19a} to R¹⁹ⁱ independently represent, at each occurrence,
   (a) H,
   (b) C₁₋₆ alkyl optionally substituted by one or more substituents selected from halo, OH, C₁₋₂ alkoxy and phenyl,
   (c) C₃₋₆ cycloalkyl optionally substituted by one or more substituents selected from halo, ═O and C₁₋₄ alkyl,
   (d) phenyl optionally substituted by halo or
   (e) Het^{f},
   provided that R^{19b} does not represent H;
(29) Het^{a} to Het^{f} independently represent 5- or 6-membered heterocyclic groups containing, as heteroatoms, one oxygen or sulfur atom and/or one to three nitrogen atoms, which heterocyclic groups may be substituted by one or more substituents selected from halo and C₁₋₄ alkyl;
(30) alkyl, alkenyl, alkynyl, cycloalkyl, cycloalkenyl, alkylene and alkenylene groups, as well as the alkyl part of alkoxy groups, may be substituted by one or more Cl or, particularly, F atoms.

Compounds of formula I that may be mentioned include those in which R⁴ and R⁵ both take the same definition (i.e. compounds in which R⁴ and R⁵ both represent H, both represent F or both represent methyl, CH₂F, CHF₂ or CF₃).

Another embodiment of the invention relates to compounds of formula I in which A represents C(O) or C(O)NH (in which latter group the NH moiety is attached to R¹) and R¹ represents:

(a) C₁₋₆ alkyl, C₂₋₆ alkenyl, C₂₋₆ alkynyl, which latter three groups are
(i) substituted by one substituent selected from C₃₋₈ cycloalkyl (optionally substituted by one or more substituents selected from halo, OH, ═O, C₁₋₆ alkyl, C₁₋₆ alkoxy and aryl), aryl and Het¹, and
(ii) optionally substituted by one or more further substituents selected from halo, CN, C₄₋₆ cycloalkyl (optionally substituted by one or more substituents selected from halo and C₁₋₄ alkyl), OR^{6a}, SR^{6b}, S(O)₂R^{6b}, S(O)₂N(H)R^{6c}, N(H)S(O)₂R^{6f}, N(R^{6g})(R^{6h}), OC(O)R⁶ⁱ C(O)OR⁶ⁱ, N(H)C(O)R⁶ⁱ, C(O)N(H)R⁶ⁱ, aryl and Het¹;
   (b) C₃₋₈ cycloalkyl or C₄₋₈ cycloalkenyl, which latter two groups are
(i) fused to one or two phenyl groups and optionally substituted by one or more substituents selected from halo, C₁₋₄ alkyl and C(O)OR⁶ⁱ, or
(ii) substituted by aryl and optionally further substituted by one or more substituents selected from halo and C₁₋₄ allyl;
   (c) aryl; or

### (d) Het³,

wherein R^{6a} to R^{6c}, R^{6f} to R⁶ⁱ aryl, Het¹ and Het³ are as defined above or below.

Yet another embodiment of the invention relates to compounds of formula I in which A represents S(O)₂ and R¹ represents:
(a) C₁₋₃ alkyl or C₁₋₃ alkenyl, which latter two groups are substituted by aryl and are optionally further substituted by one or more halo atoms;
(b) C₁₋₆ alkyl optionally substituted by one or more substituents selected from halo, OR^{6a} and S(O)₂R^{6b};
(c) C₃₋₆ monocyclic cycloalkyl optionally substituted by one or more substituents selected from halo and C₁₋₄ alkyl;
(d) C₆₋₈ bicyclic cycloalkyl optionally substituted by one or more substituents selected from halo, ═O and C₁₋₆ alkyl;
(c) aryl; or
(d) Het³,
   wherein R^{6a}, R^{6b} and Het³ are as defined above or below.

In a still further embodiment of the invention relates to compounds of formula I in which A represents C₁₋₆ alkylene and R¹ represents:
(a) C₁₋₆ alkyl or C₂₋₆ alkenyl, which latter two groups are optionally substituted by one or more substituents selected from halo and OH;
(b) C₃₋₈ cycloalkyl or C₄₋₈ (e.g. C₄₋₆) cycloalkenyl, which latter two groups are optionally substituted by one to four substituents selected from halo, ═O, OH, C₁₋₄ alkyl, O—C₁₋₄ alkyl (which latter two groups are optionally substituted by one or more halo (e.g. F) atoms) and aryl, or, particularly,
(c) aryl (e.g. naphthyl or, particularly, phenyl), or
(d) Het³,
   wherein Het³ is as defined above or below.

Particular embodiments of the invention that may be mentioned include those in which the group G-L takes any of the definitions provided at (7)(a), (c), (d), (e), (g), (h), (i), (k), (l), (m), (o) and (p) above.

Another particular embodiment of the invention that may be mentioned relates to compounds of formula I in which X represents S, in particular compounds in which X represents S and R³ represents CN or C₁₋₄ alkyl substituted by one or more fluoro atoms (e.g. CH₂F).

More particular values that may be mentioned in relation to compounds of formula I include those in which:
(1) A represents C₁₋₃ alkylene;
(2) R¹ represents
   (a) C₁₋₅ alkyl, C₂₋₄ alkenyl (which latter two groups are optionally substituted by one or more substituents selected from halo, C₆₋₈ bicyclic cycloalkyl, C₃₋₆ monocyclic cycloalkyl (which latter two groups are optionally substituted by one or more substituents selected from halo, ═O, C₁₋₄ alkyl, C₁₋₄ alkoxy and phenyl (which latter group is optionally substituted by one or more substituents selected from halo, C₁₋₄ alkyl and C₁₋₄ alkoxy)), OR^{6a}, SR^{6b}, S(O)₂R^{6b}, C(O)R⁶ⁱ, OC(O)R⁶ⁱ, C(O)OR⁶ⁱ, aryl and Het¹),
   (b) C₃₋₆ cycloalkyl or C₄₋₈ (e.g. C₄₋₆) cycloalkenyl, which latter two groups are optionally fused to one or two phenyl groups and are optionally substituted by one or more substituents selected from halo, ═O, C₁₋₄ alkyl, OR^{6a}, C(O)OR⁶ⁱ and phenyl (which latter group is optionally substituted by one or more substituents selected from halo, C₁₋₄ alkyl and C₁₋₄ alkoxy),
   (c) aryl, or
   (d) Het³;
(3) R^{6a} to R⁶ⁱ independently represent, at each occurrence,
   (a) H,
   (b) C₁₋₆ alkyl, C₂₋₄ alkenyl (which latter two groups are optionally substituted by one or more substituents selected from halo, OH, C₁₋₄ alkoxy and phenyl),
   (c) C₄₋₆ cycloalkyl (which latter group is optionally substituted by one or more substituents selected from halo and C₁₋₂ alkyl) or
   (d) phenyl (which latter group is optionally substituted by one or more substituents selected from halo, C₁₋₄ alkyl and C₁₋₄ alkoxy) provided that R^{6b} does not represent H;
(4) R² represents F or, particularly, H;
(5) R³ represents C₁₋₃ alkyl (which latter group is optionally substituted by one or more F atoms, but in a particular embodiment is unsubstituted);
(6) R⁴ and R⁵ both represent H or both represent F;
(7) the group G-L takes any of the following definitions
   (i) C(O)N(H)—C₀₋₅ alkylene-R^{a1},
   (ii) C(O)N(H)—C₀₋₃ alkylene-CH═CH—R^{a2},
   (iii) C(O)N(H)—C₁₋₃ alkylene-C═C—CH₂—R^{a3},

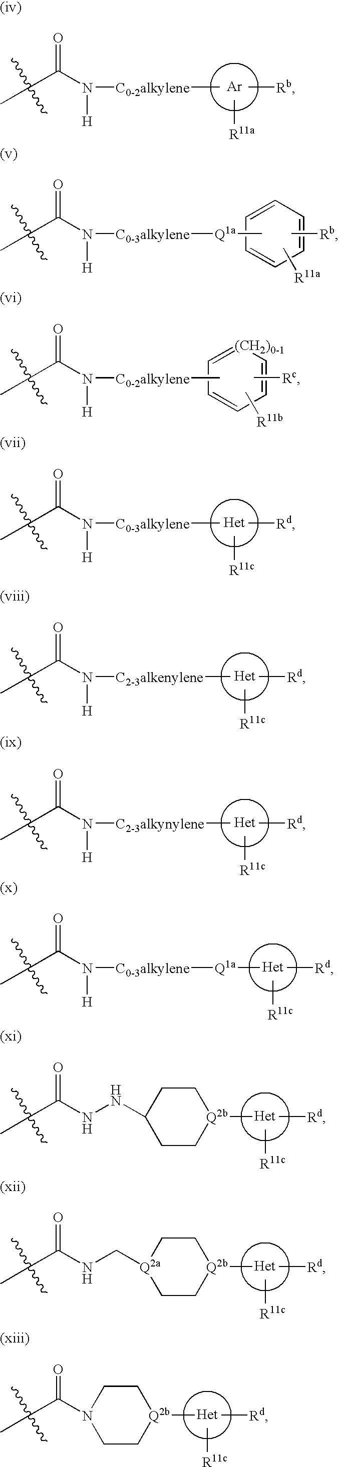

wherein Q^{1a} is as defined above;
(8) Het represents a 5- or 6-membered monocyclic, an 8-membered bicyclic, or a 9- or 10-membered ring-fused bicyclic heterocyclic group containing, as heteroatom(s), one sulfur or oxygen atom and/or one to three nitrogen atoms, which heterocyclic group
   (i) when 5- or 6-membered, is fully aromatic, fully saturated or mono-unsaturated,
   (ii) when 8-membered, is fully aromatic or, particularly, fully saturated, or
   (iii) when 9- or 10-membered, is fully aromatic or part-aromatic;
(9) R^{11a} represents H or one to three substituents selected from halo, OH, CN, C₁₋₃ alkyl and C₁₋₃ alkoxy (which latter two groups are optionally substituted by one or more substituents selected from OH, halo, C(O)OR^{12a} and C(O)N(R^{12b})R^{12c});
(10) R^{11b} represents one or two substituents selected from halo and C₁₋₃ alkyl or, particularly, R^{11b} represents H;
(11) R^{11c} represents H or one to three substituents selected from halo, OH, CN, C₁₋₃ alkyl (which latter group is optionally substituted by one or more substituents selected from halo and OH), ═O, ═NH and ═N—CN;
(12) R^{12a} to R^{12c} independently represent H, C₁₋₃ alkyl (optionally substituted by one N(R^{12e})R^{12f} group) or C₃₋₅ cycloalkyl;
(13) R^{12e} and R^{12f} independently represent H or C₁₋₂ alkyl;
(14) R^{a1}, R^{a2} and R^{a3} represent R^{a} as defined above, but particularly independently represent

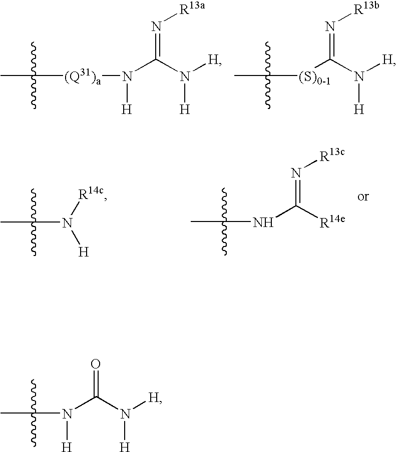

wherein Q³¹ represents O, C(O) or —CH═N— and a represents 0 or, particularly, 1;
(15) R^{b} represents
   (a) H,

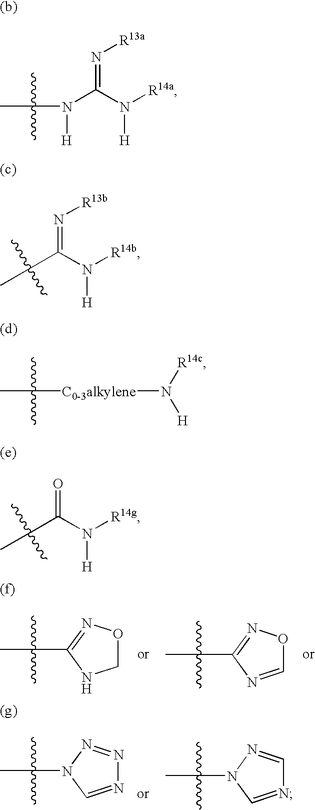

(16) R^{c} represents

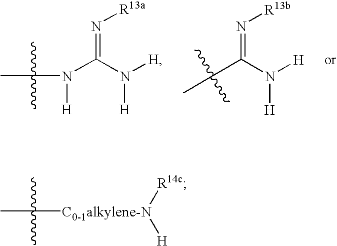

(17) R^{d} represents H,

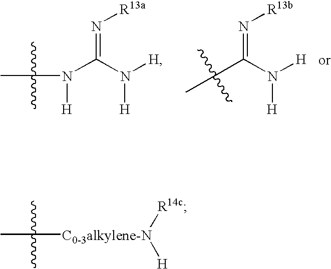

(18) R^{13a} represents H, CN, NH₂ or OR¹⁵;
(19) R^{13b} represents H, NH₂, OR¹⁵ or C(O)OR¹⁶;
(20) R^{13c} represents H or OH;
(21) R¹⁵ represents H or C₁₋₅ alkyl;
(22) R¹⁶ represents C₁₋₂ alkyl substituted by aryl;
(23) R^{10a} represents H or C₁₋₂ alkyl (which latter group is optionally substituted by OH);
(24) R^{14a} represents H, methyl, C(O)O—C₃₋₄ alkyl or C(O)OCH₂-phenyl;
(25) R^{14b} to R^{14d} and R^{14f} to R^{14g} independently represent methyl or, particularly, H,
   or R^{14c} represents
      C₁₋₂ alkyl substituted by one to three halo (e.g. F) atoms,
      C₄₋₅ cycloalkyl (e.g. cyclopentyl),
      C(O)O—C₃₋₄ alkyl or
      C(O)OCH₂-phenyl,
   or R^{14c} and R^{14d} together represent C₄ n-alkylene;
(26) R^{14e} represents H or, particularly, methyl;
(27) each aryl independently represents phenyl or naphthyl, each of which groups may be substituted by one or more substituents selected from
   (a) F, Cl, Br,
   (b) CN,
   (c) C₁₋₆ alkyl, C₂₋₃ alkenyl (which latter two groups are optionally substituted by one or more substituents selected from F, Cl, C(O)OH, C(O)OCH₃ and phenyl),
   (d) C₃₋₅ cycloalkyl,
   (e) OR^{17a},
   (f) S—C₁₋₂ alkyl, S(O)₂—C₁₋₂ alkyl (the alkyl parts of which latter two groups are optionally substituted by one or more F atoms),
   (g) S(O)₂NH₂, S(O)N(H)CH₃,
   (h) N(H)S(O)₂—C₁₋₂ alkyl (the alkyl part of which latter group is optionally substituted by one or more F atoms),
   (i) NH₂, N(H)C₁₋₂ alkyl,
   (j) CHO, C(O)—C₁₋₄ alkyl (the alkyl part of which latter group is optionally substituted by one or more F or Cl atoms), C(O)OH, C(O)O—C₁₋₄ alkyl, C(O)NH₂, C(O)N(H)—C₁₋₄ alkyl, N(H)C(O)—C₁₋₄ alkyl, N(H)C(O)O—C₁₋₄ alkyl,
   (k) phenyl (which latter group is optionally substituted by one to four substituents selected from F, Cl and Br),
   (l) Het⁹ and
   (m) Si(CH₃)₃;
(28) R^{17a} represents
   (a) H,
   (b) C₁₋₅ alkyl optionally substituted by phenyl or one or more substituents selected from F, Cl and Het¹⁰,
   (c) C₃₋₅ cycloalkyl or
   (d) phenyl optionally substituted by one to four substituents selected from F, Cl and Br;
(29) Het¹ represents a 5- to 10-membered heterocyclic group containing one to three heteroatoms selected from oxygen, nitrogen and/or sulfur, which heterocyclic group may comprise one or two rings and may be substituted by one to three substituents selected from F, Cl, Br, C₁₋₄ alkyl, ═O and OH;
(30) Het³ represents a 5- to 13-membered heterocyclic group containing one to four heteroatoms selected from oxygen, nitrogen and/or sulfur, which heterocyclic group may comprise one, two or three rings and may be substituted by one to four substituents selected from
   (a) F, Cl, Br,
   (b) C₁₋₄ alkyl (which latter group is optionally substituted by one or more substituents selected from F, Cl and OH),
   (c) C₃₋₅ cycloalkyl,
   (d) ═O,
   (e) OH, O—C₁₋₂ allyl (which latter group is optionally substituted by one or more substituents selected from F and Cl),
   (g) S(O)₂—C₁₋₂ alkyl (which latter group is optionally substituted by one or more F atoms), S(O)₂-phenyl (the phenyl part of which latter group is optionally substituted by one to four substituents selected from F, Cl, Br, methyl and methoxy),
   (h) S(O)₂NH₂, S(O)₂N(H)—C₁₋₂ alkyl,
   (i) N(O)S(O)—C₁₋₂ alkyl,
   (j) NH₂, N(H)—C₁₋₂ alkyl,
   (j) C(O)—C₁₋₄ alkyl, C(O)-phenyl (the phenyl part of which latter group is optionally substituted by one to four substituents selected from F, Cl, Br, methyl and methoxy), C(O)OH, C(O)O—C₁₋₄ alkyl, C(O)NH₂, C(O)N(H)—C₁₋₄ alkyl, N(H)C(O)—C₁₋₄ alkyl, N(H)C(O)O—C₁₋₄ alkyl,
   (l) phenyl (which latter group is optionally substituted by one to four substituents selected from F, Cl and Br) and
   (m) Het^{c};
(31) Het⁹ represents a 5- or 6-membered monocyclic heterocyclic group containing, as heteroatom(s), one sulfur or oxygen atom and/or one to three nitrogen atoms, which heterocyclic group may be substituted by one or more substituents selected from F, Cl, Br, C₁₋₄ alkyl, ═O and OH;
(32) Het¹⁰ represents a 5- or 6-membered monocyclic heterocyclic group containing, as heteroatom(s), one sulfur or oxygen atom and/or one to three nitrogen atoms, which heterocyclic group may be substituted by one or more substituents selected from F, Cl, Br, C₁₋₄ alkyl and C₁₋₄ alkoxy;
(33) Het represents a 5- or 6-membered heterocyclic group containing, as heteroatom(s), one oxygen or sulfur atom (e.g. one oxygen atom) and/or one to three (e.g. one or two) nitrogen atoms, which heterocyclic group may be substituted by one or more substituents selected from F, Cl, Br, C₁₋₄ alkyl and C₁₋₄ alkoxy.

Particular definitions of R^{a1} that may be mentioned include

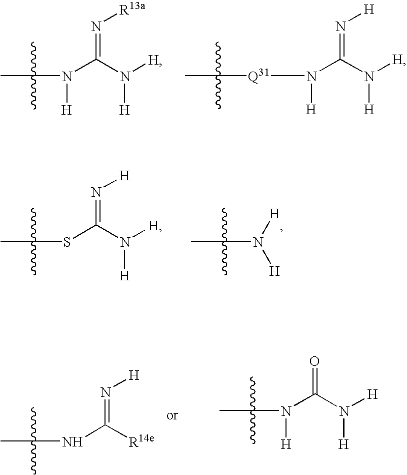

wherein R^{13a} is as defined above, but particularly represents OH, CN or NH₂ and Q³¹ and R^{14e} are as defined above.

Other particular definitions of R¹² and R¹³ that may be mentioned include —N(H)R^{14c}, wherein R^{14c} represents C₁₋₂ alkyl or, particularly, H.

Particular embodiments of the compounds of formula I that may be mentioned include those in which the group GL takes any of the following definitions.

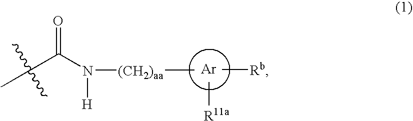

wherein aa represents 0, 1 or 2 (such as 2 or, particularly, 1);
R^{b} is as hereinbefore defined, but particularly represents tetrazol-1-yl, H,

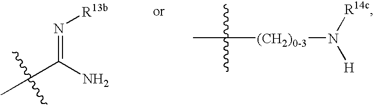

wherein R^{13b} is as hereinbefore defined, but particularly represents NH₂ or, particularly, H;
R^{14c} is as hereinbefore defined, but particularly represents C₁₋₂ alkyl optionally substituted by one to 3 F atoms (e.g. CH₂CF₃), H, cyclopentyl or C(O)O—C₃₋₄ alkyl;
R^{11a} is as hereinbefore defined, but,
(i) when R^{b} represents H, R^{11a} particularly represents one to three substituents selected from F, Cl, OH, methyl (which latter group is optionally substituted by OH or, particularly, C(O)N(R^{12b})R^{12c}) and methoxy (which latter group is substituted by C(O)N(H)R^{12b}),
(ii) when R^{b} represents —C(═NR^{13b})NH₂, R^{11a} particularly represents one or two substituents selected from F and OH or, particularly, R^{11a} represents H,
(iii) when R^{b} represents —(CH₂)₀₋₃—N(H)R^{14c}, R^{11a} particularly represents H or one or two substituents selected from F, Cl, OH, methyl, methoxy and CF₃ (e.g. a single Cl substituent).

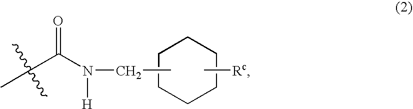

wherein R^{c} represents —C(═NR^{13b})NH₂ or, particularly, —N(H)R^{14c}, which groups are, in a particular embodiment, attached in the 4-position relative to the point of attachment of the CH₂ group;
R^{13b} and R^{14c} are as hereinbefore defined, but particularly represent H.

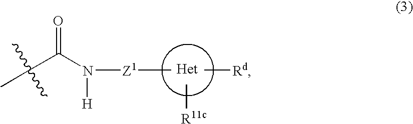

wherein Z¹ represents —CH₂C═C—, —CH═CH—, C(O)CH₂ or, particularly, C(O) or —(CH₂)_{ab}—;
when Z¹ represents —CH₂C═C—, —CH═CH—, Het represents a 5-membered, aromatic heterocyclic group containing one or, particularly, two nitrogen atoms;
when Z¹ represents C(O)CH₂, Het represents a 6-membered, fully saturated heterocyclic group containing one or, particularly, two nitrogen atoms;
when Z¹ represents C(O), Het represents a 6-membered, aromatic heterocyclic group containing two nitrogen atoms or, particularly, one nitrogen atom;
when Z¹ represents —(CH₂)_{ab}— Het represents a 5- or 6-membered monocyclic or 9 or 10-membered ring-fused bicyclic heterocyclic group containing, as heteroatom(s)
(a) a sulfur atom, or
(b) a nitrogen atom and, optionally, one or two further heteroatoms selected from nitrogen, oxygen and sulfur,
which heterocyclic group
(i) when 5- or 6-membered, is fully aromatic or fully saturated,
(ii) when 9- or 10-membered, is fully aromatic or part-aromatic;
ab represents 0 to 3, but particularly represents 1 or 2 or, when Het is 5-membered, also particularly represents 3;
R^{d} represents H, —C(═NR^{13b})NH₂ or —N(H)R^{14c}, but R^{d}, when Het is 5 or 10-membered, particularly represents —N(H)R^{14c};
R^{11c} is as hereinbefore defined, but particularly represents H or
(I) when Het is 6-membered and aromatic (e.g. a pyridinyl group), one or two substituents selected from F, Cl, methyl and CH₂OH,
(II) when Het is 6-membered and fully saturated, a methyl or a ═NH substituent;
R^{13b} is as hereinbefore defined, but particularly represents H;
R^{14c} is as hereinbefore defined, but particularly represents H or, when Het is 6-membered, methyl.

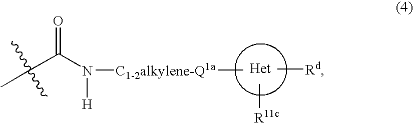

wherein Q^{1a} represents O or NR^{10a};
R^{10a} represents H, methyl or —CH₂CH₂OH;
Het represents a 6-membered or 10-membered, aromatic heterocyclic group containing two nitrogen atoms or, particularly, one nitrogen atom;
R^{d} represents H or —N(H)R^{14c};
R^{14c} is as hereinbefore defined, but particularly represents H;
R^{11c} is as hereinbefore defined, but particularly represents H or, when Het contains two nitrogen atoms, represents Cl.

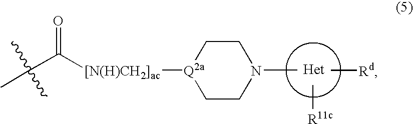

wherein Q^{2a} represents N or CH;
ac represents 0 or 1, but, when Q^{2a} represents CH, particularly represents 1;
Het represents a 6-membered, aromatic heterocyclic group containing two nitrogen atoms or, particularly, one nitrogen atom (e.g. a pyridinyl group, such as a pyridin-4-yl group);
R^{d} and R^{11c} are as hereinbefore defined, but particularly represent H;

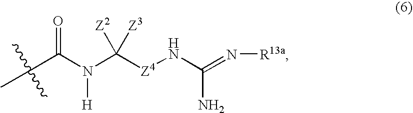

wherein Z² and Z³ independently represent H or F, but, particularly, Z² and Z³ both represent H or both represent F;
Z⁴ represents —(CH₂)₂C(O)— or, particularly, —CH₂C(O)—, —CH₂O—, —CH₂—C(H)═N— or —C(H)═N—;
R^{13a} is as hereinbefore defined, but particularly represents H.

In another embodiment of the invention, the compound of formula I is a compound of formula Ia,

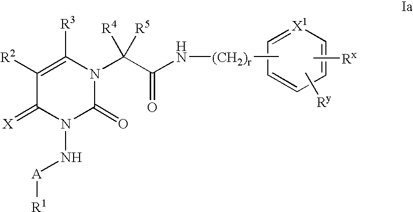

wherein X¹ represents CH or N;
when X¹ represents CH
(a) R^{x} takes the same definitions as R^{b} above, and
(b) R^{y} takes the same definitions as R^{11a} above;
   when X¹ represents N
(a) R^{x} takes the same definitions as R^{d} above, and
(b) R^{y} takes the same definitions as R^{11c} above;
   r represents 1 to 3; and
   R¹ to R⁵, R^{11a}, R^{11c}, R^{b}, R^{d}, A and X are as defined above,
   which compounds are also referred to hereinafter as “the compounds of the invention”.

Particular values that may be mentioned in relation to compounds of formula Ia include those in which:

when X¹ represents CH, R^{x} represents tetrazol-1-yl, H or (CH₂)₁₋₂N(H)R^{14c} (e.g. CH₂N(H)R^{14c});
when X¹ represents N, R^{x} represents H or —N(H)R^{14c};
when X¹ represents CH, R^{y} represents H or one to three substituents selected from halo, C₁₋₂ alkyl, C₁₋₂ alkoxy (which latter two groups are optionally substituted by one or more F atoms), OH, CH₂OH and OCH₂C(O)N(H)R^{12b};
when X¹ represents N, R^{y} represents H or one to three substituents selected from halo and C₁₋₂ alkyl;
R^{12b} represents H or, particularly, C₁₋₃ alkyl optionally substituted by N(CH₃)₂ (e.g. ethyl or (CH₂)₂₋₃N(CH₃)₂, particularly (CH₂)₃N(CH₃)₂);
r represents 2 or, particularly, 1.

More particular values that may be mentioned in relation to compounds of formula Ia include those in which:

A represents C₁₋₃ alkylene optionally substituted by one or more F atoms;
R¹ represents
(a) C₁₋₃ alkyl substituted by phenyl (which latter group is optionally substituted by one or more substituents selected from halo, C₁₋₄ alkyl and C₁₋₄ alkoxy (which latter two groups are optionally substituted by one or more F atoms)),
(b) phenyl or naphthyl (which latter two groups are optionally substituted by one or more substituents selected from CN, halo, C₁₋₄ alkyl, C₁₋₄ alkoxy (which latter two groups are optionally substituted by one or more F atoms), O-phenyl, O—CH₂-Het¹⁰ and Het⁹,
(c) a 5- or 6-membered monocyclic (e.g. aromatic) heterocyclic group containing, as heteroatom(s), an oxygen or sulfur atom and/or one to three nitrogen atoms, which heterocyclic group is optionally substituted by one to four substituents selected from F, Cl, Br, ═O, OH, C₁₋₄ alkyl (which latter group is optionally substituted by one or more halo atoms or by OH), C₁₋₄ alkoxy, S(O)₂-phenyl, C(O)-phenyl, phenyl and Het^{c},
(d) a 9- or 10-membered bicyclic (e.g. part-aromatic) heterocyclic group containing one to three heteroatoms selected from oxygen, nitrogen and/or sulfur (e.g. two oxygen atoms), which heterocyclic group is optionally substituted by one to four substituents selected from F, Cl, Br, C₁₋₄ alkyl and C₁₋₄ alkoxy,
(e) C₁₋₅ alkyl, or
(f) C₄₋₇ cycloalkyl or C₅₋₇ cycloalkenyl, which latter two groups are optionally substituted by one or more methyl groups;
   Het⁹ represents a 5- or 6-membered monocyclic heterocyclic group containing, as heteroatom(s), one sulfur or oxygen atom and/or one or two nitrogen atoms, which heterocyclic group may be substituted by one to three substituents selected from F, Cl and methyl;
   Het¹⁰ represents a 5- or 6-membered monocyclic, aromatic heterocyclic group containing, as heteroatom(s), one sulfur or oxygen atom and/or one or two nitrogen atoms, which heterocyclic group may be substituted by one to three substituents selected from F, Cl, methyl and methoxy;
   Het^{c} represents a 5- or 6-membered monocyclic heterocyclic group containing, as heteroatom(s), an oxygen or sulfur atom and/or or one or two nitrogen atoms, which heterocyclic group is optionally substituted by one to four substituents selected from F, Cl, Br, C₁₋₄ alkyl and C₁₋₄ alkoxy;
   R³ represents methyl (which latter group is optionally substituted by one or more F atoms, providing, for example, CHF);
   R⁴ and R⁵ both represent H;
   when X¹ represents CH and R^{x} represents H, then R^{y} represents one to three substituents selected from OH, methyl, CH₂OH, OCH₂C(O)N(H)R^{12b} and halo (particularly one to three halo atoms (e.g. one to three Cl atoms, such as two Cl atoms attached in the 2- and 5-positions relative to the point of attachment of the (CH₂)ᵣ group));
   when X¹ represents CH and R^{x} represents (CH₂)₁₋₂N(H)R^{14c}, then R^{y} represents H or, particularly, one or two substituents selected from halo, C₁₋₂ alkyl and C₁₋₂ alkoxy (which latter two groups are optionally substituted by one or more F atoms) (and particularly R^{y} represents one or two halo atoms (e.g. one or two Cl atoms, such as a Cl atom attached in the 3-position relative to the point of attachment of the (CH₂)ᵣ group));
   when X¹ represents CH and R^{x} represents tetrazol-1-yl, then R^{y} represents H or one or two halo (e.g. Cl atoms);
   when X¹ represents CH, the group (CH₂)₁₋₂N(H)R^{14c}, if present, is attached at the 5-position or, particularly, the 6-position relative to the point of attachment of the (CH₂)ᵣ group;
   when X¹ represents CH, the tetrazol-1-yl group, if present, is attached at the 6 position relative to the point of attachment of the (CH₂)ᵣ group;
   when X¹ represents N and R^{x} represents H, R^{y} represents H or, particularly, one or two substituents selected from halo (e.g. F) and methyl;
   when X¹ represents N and R^{x} represents —N(H)R^{14c}, R^{y} represents H or one or two methyl groups;
   R^{14c} represents CH₂CF₃, cyclopentyl or C(O)O—C₄ alkyl or, particularly, H.

Still more particular values that may be mentioned in relation to compounds of formula Ia include those in which:

A represents C₁₋₃ (e.g. C₁₋₂) alkylene (optionally gem-disubstituted by two F atoms);
R¹ represents
(a) C₁₋₂ alkyl substituted by phenyl (which latter group is optionally substituted by one or more substituents selected from F, Cl and Br), or
(b) phenyl (which latter group is optionally substituted by one or more substituents selected from F, Cl, Br, CN, C₁₋₃ alkyl, C₁₋₃ alkoxy (which latter group two groups are optionally substituted by one or more F atoms (thus forming, for example, C₁₋₂ alkyl, CF₃, C₁₋₂ alkoxy or OCF₃)), O-phenyl, O—CH₂-Het¹⁰ and Het⁹),
(c) naphthyl (e.g. 1-naphthyl), or
(d) pyridinyl (e.g. pyridin-2-yl or pyridin-3-yl) optionally substituted by one or two substituents selected from F, Cl, (N-)oxo, OH, C₁₋₄ alkyl (such as methyl, which C₁₋₄ alkyl group is optionally substituted by one or more halo atoms or by OH) or, particularly, C₁₋₄ alkoxy (e.g. tert-butoxy or methoxy) or Het^{c},
(e) pyridonyl (e.g. 2-pyridon-3-yl) optionally substituted by one or two substituents selected from F, Cl, and C₁₋₄ alkyl (e.g. methyl);
(f) pyrazinyl (e.g. pyrazin-2-yl) optionally substituted by one or two substituents selected from F, Cl and methyl;
(g) a 5-membered aromatic heterocyclic group containing, as heteroatom(s), an oxygen or sulfur atom and/or one to three nitrogen atoms (e.g. imidazolyl, isoxazolyl, pyrazolyl, pyrrolyl, thiazolyl, or thienyl), which heterocyclic group is optionally substituted by one to four (e.g. one to three) substituents selected from F, Cl, C₁₋₄ alkyl (e.g. methyl or ethyl), C₁₋₄ alkoxy (e.g. methoxy), S(O)₂-phenyl, C(O)-phenyl, phenyl, morpholinyl (e.g. morpholin-4-yl), 1,3,4-triazolyl (e.g. 1,3,4-triazol-1-yl), thienyl (e.g. 2-thienyl) and pyridinyl (e.g. pyridin-2-yl),
(h) 2,3-dihydrobenzofuranyl, benzomorpholinyl, benzodioxanyl, 2,1,3-benzoxadiazolyl, or, particularly, benzodioxolyl or quinolinyl, all of which groups are optionally substituted by one or more (e.g. one to three) substituents selected from F, Cl, C₁₋₂ alkyl and C₁₋₂ alkoxy,
(i) C₁₋₄ alkyl (e.g. isopropyl or tert-butyl), or
(j) cyclopentyl, cyclohexyl or C₇ bicyclic cycloalkenyl (e.g. bicyclo[2.2.1]heptene, which latter three groups are optionally substituted by one to four methyl groups;
   Het⁹ represents a 6-membered, saturated, monocyclic heterocyclic group containing, as heteroatom(s), one oxygen atom and/or one or two nitrogen atoms, which heterocyclic group may be substituted by one or two methyl substituents;
   Het¹⁰ represents a 5-membered, monocyclic, aromatic heterocyclic group containing, as heteroatom(s), one sulfur or oxygen atom and/or one or two nitrogen atoms, which heterocyclic group may be substituted by one to three substituents selected from Cl and methyl;
   Het^{c} represents a 6-membered, saturated, monocyclic heterocyclic group containing, as heteroatom(s), one oxygen atom and/or one or two nitrogen atoms, which heterocyclic group may be substituted by one or two methyl substituents;
   R³ represents methyl;
   X¹ represents CH or N (e.g. CH);
   when X¹ represents CH, R^{x} represents tetrazol-1-yl or, particularly, CH₂N(H)R^{14c} (which latter two groups are attached, for example, in the 6-position relative to the point of attachment of the (CH₂)ᵣ group);
   R^{x} may alternatively represent H when X¹ represents CH and R^{y} represents one to three substituents selected from OH, methyl, CH₂OH, OCH₂C(O)N(H)R^{12b} and halo;
   R^{14c} represents H.

Yet further particular values that may be mentioned in relation to compounds of formula Ia include those in which:

A represents CH(CH₃)CH₂ (in which latter group the CH(CH₃) unit is attached to R¹) or, particularly, CH₂, (CH₂)₂ or CF₂CH₂ (in which latter group the CF₂ unit is attached to R¹);
R¹ represents
(a) isopropyl or tert-butyl,
(b) cyclopentyl, cyclohexyl or bicyclo[2.2.1]hept-5-ene,
(c) phenyl optionally substituted by one or two substituents selected from halo (e.g. F or Cl), CN, methyl, CF₃, methoxy, OCF₃, phenoxy, morpholin-4-yl or O—CH₂-(2-chlorothiazol-5-yl),
(d) imidazolyl optionally substituted by one to three substituents selected from Cl, methyl and phenyl,
(e) isoxazolyl (e.g. isoxazol-3-yl or isoxazol-4-yl) optionally substituted by one or two substituents selected from methyl, phenyl and 2-thienyl,
(f) thiazolyl (e.g. thiazol-5-yl) optionally substituted by one or two methyl groups,
(g) thienyl (e.g. thien-2-yl) optionally substituted by Cl or pyridinyl (e.g. pyridin-2-yl),
(h) pyrazolyl (e.g. pyrazol-4-yl) optionally substituted by one to three substituents selected from Cl, methyl, ethyl, phenyl and morpholin-4-yl,
(i) pyrrolyl (e.g. pyrrol-2-yl or pyrrol-3-yl) optionally substituted by one to three substituents selected from methyl, S(O)₂-phenyl, C(O)-phenyl and 1,3,4-triazol-1-yl,
(j) pyridinyl (e.g. pyridin-2-yl or pyridin-3-yl) optionally substituted by OH, methoxy or morpholin-4-yl, and optionally in the form of an N-oxide,
(k) pyridonyl (e.g. 2-pyridon-3-yl),
(l) pyrazinyl (e.g. pyrazin-2-yl),
(m) benzodioxolyl (e.g. 5-benzodioxolyl) optionally substituted by halo (e.g. Cl),
(n) benzomorpholinyl (e.g. 7-benzomorpholinyl) optionally substituted by methyl;
(o) 2,1,3-benzoxadiazolyl (e.g. 2,1,3-benzoxadiazol-5-yl),
(p) 2,3-dihydrobenzofuranyl (e.g. 2,3-dihydrobenzofuran-5-yl) or
(q) quinolinyl (e.g. 8-quinolinyl);
   the group

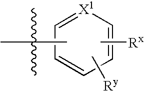

represents

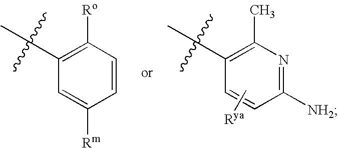

R^{o} represents H, F, Cl, OH, methyl or, particularly, tetrazol-1-yl, OCH₂C(O)N(H)R^{12b} or CH₂N(H)R^{14c};
R^{m} represents H, methyl, CF₃, methoxy, F or, particularly, Cl (for example:
   (a) when R^{o} represents H or Cl, then R^{m} represents Cl;
   (b) when R^{o} represents OH or methyl, then R^{m} represents F or, particularly Cl; and
   (c) when 9 represents tetrazol-1-yl, OCH₂C(O)N(H)R^{12b} or CH₂N(H)R^{14c} then R^{m} represents H, methyl, CF₃, methoxy, F or, particularly, Cl);
      R^{ya} represents H or, particularly, methyl.

Still further particular values that may be mentioned in relation to compounds of formula Ia include those in which

A represents (CH₂)₂ or, particularly, CH₂ or CF₂CH₂ (in which latter group the CF₂ unit is attached to R¹);
R¹ represents:
(a) phenyl optionally substituted by one or two substituents selected from halo (e.g. F or Cl) and methyl (e.g. phenyl substituted by one or two substituents selected from F and Cl),
(b) isoxazol-4-yl optionally substituted by one or two methyl substituents,
(c) pyrazol-4-yl optionally substituted by one to three substituents selected from Cl and methyl, or, particularly,
(d) pyridinyl (e.g. pyridin-3-yl or, particularly, pyridin-2-yl) optionally substituted by OH or halo (e.g. F or Cl), but in a particular embodiment is unsubstituted;
   the group

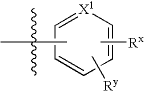

represents

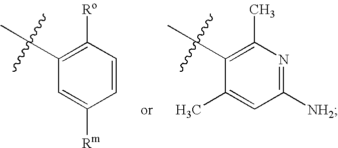

R^{o} represents tetrazol-1-yl, OCH₂C(O)N(H)R^{12b} or CH₂NH₂;
R^{m} represents H or, particularly, Cl;
R^{12b} represents C₁₋₃ alkyl (e.g. ethyl).

For the avoidance of doubt, the particular definitions of groups given above in relation to compounds of formula Ia are also, where relevant, particular definitions of the equivalent groups in compounds of formula I. Moreover, references herein to compounds of formula I also include, where relevant, references to compounds of formula Ia.

Particular embodiments of the invention that may be mentioned include the compounds of the Examples disclosed hereinafter.

### Preparation

Compounds of formula I (including compounds of formula Ia) may be made in accordance with techniques well known to those skilled in the art, for example as described hereinafter.

According to a further aspect of the invention there is provided a process for the preparation of a compound of formula I, which comprises:

(a) for compounds of formula I in which R^{7a} and R^{7b} together represent ═O, coupling of a compound of formula II,

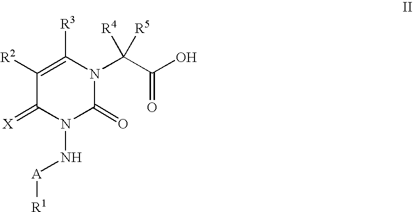

wherein R¹ to R⁵, A and X are as hereinbefore defined, with a compound of formula III,

H-G^{a}-L III

wherein L is as hereinbefore defined and G^{a} represents
(i) —N(R^{8a})—[CH(C(O)R⁹)]₀₋₁—C₀₋₃ alkylene-(Q¹)ₐ-,
(ii) —N(R^{8b})—C₂₋₃ alkenylene-(Q¹)ₐ-,
(iii) —N(R^{8b})—C₂₋₃ alkynylene-(Q¹)ₐ-,

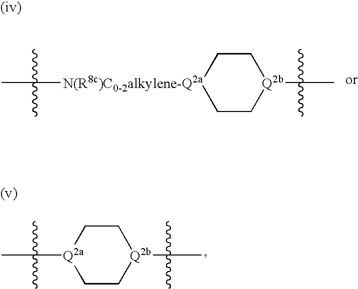

wherein Q^{2a} represents N or NHCH and R^{8a}, R^{8b}, R^{8c}, R⁹, Q¹, Q^{12b} and a are as hereinbefore defined,
   for example in the presence of a coupling agent (e.g. oxalyl chloride in DMF, EDC, DCC, HBTU, HATU, PyBOP or TBTU), an appropriate base (e.g. pyridine, DMAP, TEA, 2,4,6-collidine or DIPEA) and a suitable organic solvent (e.g. DCM, MeCN, EtOAc or DMF);
   (b) for compounds of formula I in which R^{7a} and R^{7b} independently represent H or methyl, reaction of a compound of formula IV,

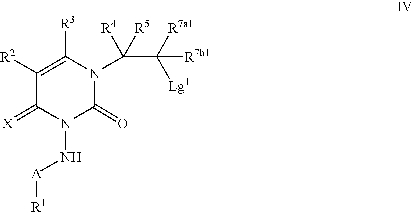

wherein R^{7a1} and R^{7b1} independently represent H or methyl, Lg¹ represents a suitable leaving group (e.g. halo or OS(O)R′, wherein R′ represents, for example, C₁₋₄ alkyl, C₁₋₄ perfluoroalkyl, phenyl, toluoyl or benzyl) and R¹ to R⁵, A and X are as hereinbefore defined, with a compound of formula III, as hereinbefore defined, for example under conditions known to those skilled in the art (such as in the presence of a suitable solvent (e.g. MeCN or DMF) and optionally in the presence of an appropriate base (e.g. TEA or pyridine optionally mono-di- or tri-substituted by C₁₋₄ alkyl) and/or a catalyst (such as NaI));
(c) for compounds of formula I in which R^{7a} represents H and R^{7b} represents H or methyl, reaction of a compound of formula V,

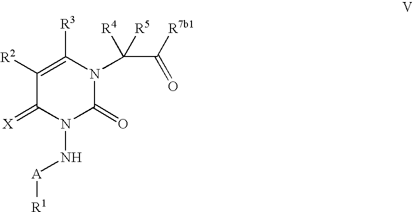

wherein R⁴ to R⁵, R^{7b1}, A and X are as hereinbefore defined, with a compound of formula III, as hereinbefore defined, for example under conditions known to those skilled in the art (such as at between ambient temperature and reflux in the presence of a suitable solvent (e.g. ethanol, methanol, acetic acid or binary mixtures thereof), followed by reduction in the presence of a reducing agent (e.g. NaBH₃CN or NaB(OAc)₃H), for example under conditions known to those skilled in the art (e.g. at ambient temperature (such as 15 to 25° C.) in the presence of a suitable solvent (such as ethanol);
(d) for compounds of formula I in which G represents

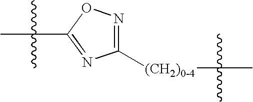

and L represents L^{a}, which latter group represents L as hereinbefore defined, except that it does not represent C₀ alkylene-R^{a}, cyclisation of a compound of formula VI,

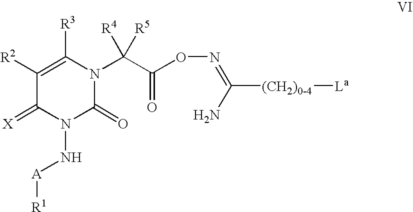

wherein R¹ to R⁵, A, X and L^{a} are as hereinbefore defined, for example at elevated temperature (e.g. 60° C. to reflux) in the presence of a suitable solvent (e.g. pyridine, toluene, 1,4-dioxane or THF) and optionally in the presence of a suitable catalyst (e.g. (n-Bu)₄NF, which may particularly be employed when the reaction solvent is THF);
(e) for compounds of formula I in which R^{a}, R^{b}, R^{c} or R^{d} represents —C(═NH)NH₂, —C(═NNH₂)NH₂ or —C(═NOH)NH₂, reaction of a compound of formula VII,

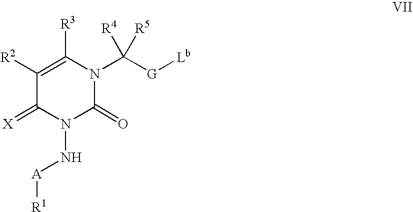

wherein L^{b} represents L as hereinbefore defined, except that R^{a}, R^{b}, R^{c} or R^{d} (as appropriate) is replaced by a cyano or —C(—NH)O—C₁₋₄ alkyl group, and R¹ to R⁵, A, G and X are as hereinbefore defined, with a suitable source of ammonia, hydrazine or hydroxylamine (e.g. ammonia gas, ammonium acetate, hydrazine, hydrazine monohydro-chloride, hydroxylamine or hydroxylamine hydrochloride) under conditions known to those skilled in the art (e.g. conditions such as those described in Tetrahedron Lett. 40, 7067 (1999)), for example from ambient (e.g. 15 to 25° C.) to elevated temperature (e.g. 60° C. to reflux) in the presence of a suitable solvent (e.g. ethanol);
(f) for compounds of formula I in which R^{13a}, R^{13b} or R^{13c} represents H, deprotection of a corresponding compound of formula I in which R^{13a}, R^{13b} or R^{13c} (as appropriate) represents C(O)O—CH₂aryl (e.g. C(O)O-benzyl), for example under conditions known to those skilled in the art (such as hydrogenation in the presence of an appropriate catalyst (e.g. Pt/C or, particularly, Pd/C), a suitable solvent (e.g. an alcohol such as ethanol or, particularly, methanol) and, optionally, an acid (e.g. HCl));
(g) for compounds of formula I in which R^{14c} represents H, deprotection of a corresponding compound of formula I in which R^{14c} represents C(O)O—C₁₋₆ alkyl (e.g. C(O)O-tert-butyl), for example under conditions known to those skilled in the art (e.g. acid or base hydrolysis, such as, for deprotection of compounds in which R^{14c} represents C(O)O-tert-butyl, reaction with HCl gas in the presence of a suitable solvent (e.g. an alcohol such as ethanol or, particularly, methanol), or reaction with trifluoroacetic acid at sub-ambient temperature (e.g. 0 to 4° C.), optionally in the presence of a suitable solvent such as DCM);
(h) reaction of a compound of formula VIII,

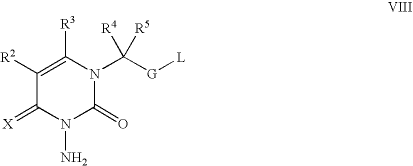

wherein R² to R⁵, G, L and X are as hereinbefore defined, with a compound of formula IX,

R¹-A-Lg² IX

wherein Lg² represents a suitable leaving group (e.g. halo, trifluoromethane-sulfonate or OH) and R¹ and A are as hereinbefore defined, for example under conditions known to those skilled in the art (such as in the presence of an appropriate base (e.g. K₂CO₃, pyridine or 2,6-di-tert-butyl-4-methylpyridine) and a suitable solvent (e.g. DCM or 1,2-dichloroethane));
(i) for compounds of formula I in which A represents C(O)NH, reaction of a compound of formula VIII, as hereinbefore defined, with a compound of formula VIII,

R¹—N═C═O X

wherein R¹ is as hereinbefore defined, for example under conditions known to those skilled in the art (such as at ambient temperature (e.g. 15 to 25° C.) in the presence of a suitable solvent (e.g. DCM));
(j) for compounds of formula I in which A represents C₁₋₆ alkylene, reaction of a compound of formula VIII, as hereinbefore defined, with a compound of formula

R¹—C₀₋₅alkylene-CHO XI

wherein R¹ is as hereinbefore defined, for example under conditions known to those skilled in the art (such as those described at process alternative (c) above) followed by reduction in the presence of a reducing agent (e.g. as described in process alternative (c) above);
(k) for compounds of formula I in which R^{a}, R^{b}, R^{c} or R^{d} represents —C(═NCN)NH₂, reaction of a corresponding compound of formula I in which R^{a}, R^{b}, R^{c} or R^{d}, respectively, represents —C(═NH)NH₂ with cyanogen bromide, for example under conditions known to those skilled in the art (e.g. in the presence of a suitable base (such as an alkali metal alkoxide like sodium ethoxide) and an appropriate solvent (such as a lower alkyl alcohol like ethanol);
(l) reaction of a compound of formula XII,

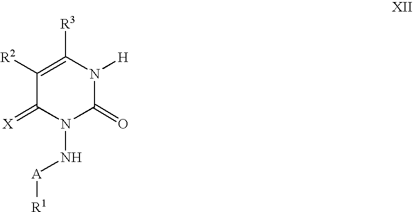

wherein R¹, R², R³, A and X are as hereinbefore defined, with a compound of formula XIII,

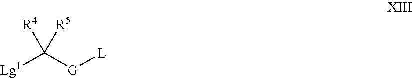

wherein R⁴, R⁵, Lg¹, G and L are as hereinbefore defined, in the presence of a base (such as triethylamine, NaH or Na₂CO₃), for example under conditions known to those skilled in the art (e.g. at between ambient and reflux temperatures in the presence of a suitable solvent (such as DCM, MeCN, THF or DMF)); or
(m) reaction of a compound of formula XII, as hereinbefore defined, with a compound of formula XIV,

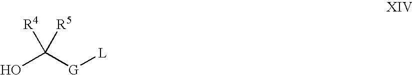

wherein R⁴, R⁵, G and L are as hereinbefore defined, under Mitsunobu conditions, for example in the presence of a suitable dehydrating agent (such as a phosphine (e.g. triphenylphosphine) in combination with an electron-poor diazo compound (e.g. DEAD)).

Compounds of formula II may be prepared by hydrolysis of a compound of formula XV,

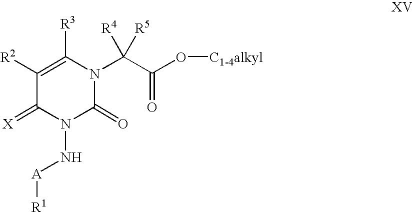

wherein R¹ to R⁵, A and X are as hereinbefore defined, e.g. under conditions known to those skilled in the art (for example: (i) when the C₁₋₄ alkyl group is other than tert-butyl, by basic hydrolysis in the presence of an alkali metal hydroxide (e.g. LiOH or, particularly, NaOH) and a suitable solvent (e.g. water, THF, methanol or a mixture thereof); or (ii) when the C₁₋₄ alkyl group is tert-butyl, by acidic hydrolysis performed, for example, by reaction at ambient temperature with an appropriate volume of ethyl acetate that has saturated with hydrogen chloride gas).

Compounds of formula IV may be prepared by procedures known to those skilled in the art, such as procedures analogous to those described in WO 2005/040137. For example:

(1) for compounds of formula IV in which Lg¹ represents halo, reaction of a corresponding compound of formula XVI,

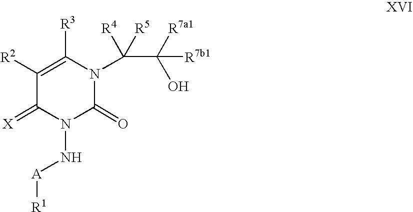

wherein R¹ to R⁵, R^{7a}, R^{7b}, A and X are as hereinbefore defined, with a halogenating agent (such as oxalyl chloride, SOCl₂, SOBr₂, PCl₃, PBr₃, PCl₅, PBr₅, triphenylphosphine dibromide or combinations of: (i) triphenylphosphine or bis(diphenylphosphino)ethane with the halogen (e.g. bromine or iodine); or (ii) triphenylphosphine with CCl₄, CBr₄, hexachloroethane or hexachloroacetone) under conditions known to those skilled in the art; or
(2) for compounds of formula IV in which Lg¹ represents OS(O)₂R′, reaction of a corresponding compound of formula XVI, as hereinbefore defined, with a compound of formula XVII,

R′S(O)₂Cl XVII

wherein R′ is as hereinbefore defined, for example under conditions known to those skilled in the art (such as in the presence of a suitable base (e.g. TEA, pyridine or N,N-diisopropylethylamine) and an appropriate solvent (e.g. DCM or MeCN)).

Compounds of formula V may be prepared by oxidation of a corresponding compound of formula XVI, as hereinbefore defined except that R^{7a1} represents H, in the presence of a suitable oxidising agent, for example under conditions known to those skilled in the art, such as reaction with PCC, oxalyl chloride and DMSO (Swern oxidation) or, particularly, Dess-Martin periodinane in the presence of a suitable solvent (such as DCM).

Compounds of formula VI may be prepared by the coupling of a compound of formula II, as hereinbefore defined, with a compound of formula XVIII,

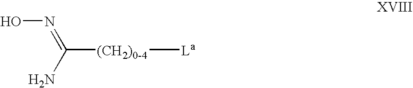

wherein L^{a} is as hereinbefore defined, for example under conditions well know to those skilled in the art (e.g. those described in WO 01/79262, such as at ambient temperature (e.g. 15 to 25° C.) in the presence of a coupling agent (e.g. EDC) and a suitable solvent (e.g. DMF)).

As the skilled person will appreciate, in some instances, compounds of formula VII are identical to certain compounds of formula I (e.g. compounds in which R^{b}, R^{c} or R^{d} represents H and R^{11a}, R^{11b} or R^{11c}, respectively, represents CN). In this respect, compounds of formula VII may be prepared by analogy with the procedures described herein for the preparation of compounds of formula I.

Compounds of formula VIII in which X represents O may be prepared by reduction of a compound of formula XIX,

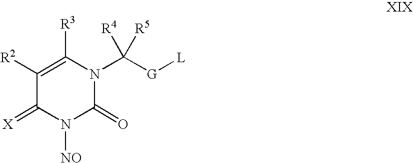

wherein R², R³, R⁴, R⁵, G and L are as hereinbefore defined, for example under conditions that are well known to those skilled in the art (such as by reaction with zinc metal (e.g. zinc powder or iron metal powder) in the presence of an appropriate acid (e.g. acetic acid or hydrochloric acid) and optionally in the presence of a suitable solvent (e.g. methanol)).

Compounds of formula VIII may alternatively be prepared by reaction of a compound of formula XX,

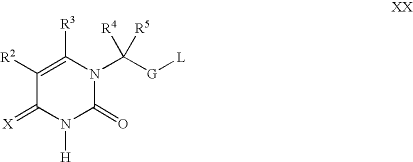

wherein R², R³, R⁴, R⁵, G, L and X are as hereinbefore defined, with O-(diphenylphosphinyl)hydroxylamine or O-(2,4-dinitrophenyl)hydroxylamine, for example under conditions known to those skilled in the art (e.g. at ambient temperature (such as 15 to 25° C.) in the presence of an appropriate base (such as Cs₂CO₃ or NaH) and a suitable solvent (such as DMF)).

Compounds of formula XI may be prepared by oxidation of an alcohol of formula XXI,

R¹—C₀₋₅alkylene-CH₂OH XXI

wherein R¹ is as hereinbefore defined, for example under conditions known to those skilled in the art, such as those described above in relation to the synthesis of compounds of formula V.

Compounds of formula XX may be prepared by analogy with compounds of formula I (see, for example, process alternatives (h) to (j) above).

Compounds of formula XV may be prepared by reaction of a compound of formula XXII,

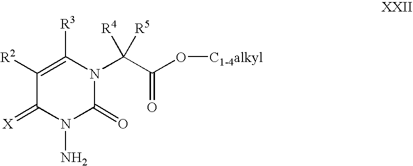

wherein R², R³, R⁴, R⁵ and X are as hereinbefore defined, with a compound of formula IX, X or XI as hereinbefore defined, for example under conditions known to those skilled in the art (e.g. conditions described at process alternatives (h), (i) and (j) above in respect of compounds of formula I).

Compounds of formula XVI in which R^{7a1} and R^{7b1} both represent H may be prepared by reduction of a corresponding compound of formula II or XV, as hereinbefore defined, in the presence of a suitable reducing agent (e.g. a reagent based upon an aluminium or boron hydride, such as LiAlH₄, LiBH₄, borane or diborane), for example under conditions known to those skilled in the art (such as conditions analogous to those disclosed in WO 2005/040137, e.g. reaction at ambient temperature in the presence of a suitable solvent (such as THF)).

Compounds of formula XVIII may be prepared by methods well known to those skilled in the art. For example, compounds of formula XVIII may be prepared by reaction of a compound of formula XXIII or XXIV,

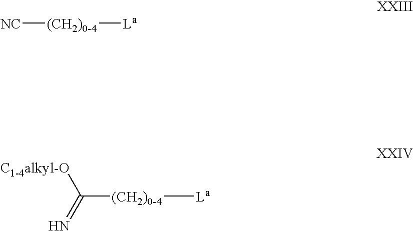

wherein L^{a} is as hereinbefore defined, with hydroxylamine or an acid addition salt thereof, for example under conditions described at process step (c) above in respect of compounds of formula I.

Compounds of formula XIX may be prepared by nitrosation of a corresponding compound of formula XX, as hereinbefore defined, for example under conditions well known b those skilled in the art, e.g. reaction at with a nitrosating agent (such as nitrous acid, NOCl, N₂O₃, N₂O₄ or, particularly, a C₁₋₆ alkyl nitrite (e.g. tert-butyl nitrite)) in the presence of a suitable solvent (e.g. diethyl ether) and optionally in the presence of an appropriate base (e.g. pyridine).

Compounds of formula XX may be prepared by analogy with compounds of formulae I and XXVII.

Compounds of formula XXI may be prepared by reduction of a carboxylic acid of formula XXV,

R¹—C₀₋₅alkylene-C(O)OH XXV

wherein R¹ is as hereinbefore defined, for example under conditions known to those skilled in the art, such as reaction with LiAlH₄ or, particularly, borane in the presence of a suitable solvent (such as THF).

Compounds of formula XXII in which X represents O may be prepared by reduction of a compound of formula XXVI,

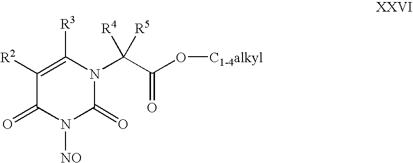

wherein R², R³, R⁴ and R⁵ are as hereinbefore defined, for example under conditions described hereinbefore in respect of the preparation of compounds of formula VIII.

Compounds of formula XXII may alternatively be prepared by reaction of a compound of formula XXVII,

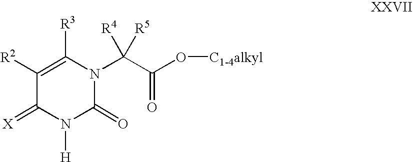

wherein R², R³, R⁴, R⁵ and X are as hereinbefore defined, with O-(diphenylphosphinyl)hydroxylamine or O-(2,4-dinitrophenyl)hydroxylamine, for example under conditions described hereinbefore in respect of the preparation of compounds of formula VI.

Compounds of formula XXVI may be prepared by nitrosation of a corresponding compound of formula XXVII, as hereinbefore defined, for example under conditions described hereinbefore in respect of the preparation of compounds of formula XIX.

Compounds of formula XXVII in which X represents S may be prepared by reaction of a corresponding compound of formula XXVII in which X represents O with P₂S₅ or Lawesson's reagent, for example at between ambient and reflux temperature in the presence of a suitable solvent (such as trichloroethylene or dioxane).

Compounds of formula XXVII in which X represents O, R² represents H and R³ represents C₁₋₆ alkyl optionally substituted by one or more F atoms may be prepared by reaction of a corresponding compound of formula XXVIII,

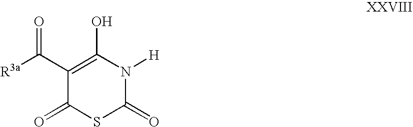

wherein R^{3a} represents C₁₋₆ alkyl optionally substituted by one or more F atoms, with a compound of formula XXIX,

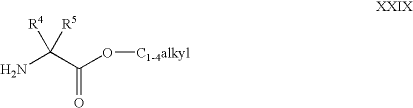

wherein R⁴ and R⁵ are as hereinbefore defined, for example under conditions known to those skilled in the art, such as reaction at between ambient and reflux temperatures in the presence of a solvent and/or a base (e.g. pyridine).

Compounds of formula XXVII may alternatively be prepared by reaction of a compound of formula XXX,

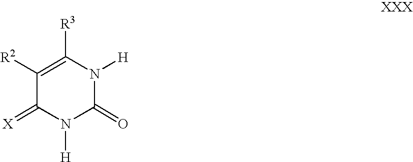

wherein R², R³ and X are as hereinbefore defined, with a compound of formula XXXI,

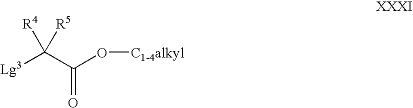

wherein Lg³ represents a suitable leaving group (e.g. halo or OS(O)₂R′, wherein R′ is as hereinbefore defined) or Lg³ represents OH, and R⁴ and R⁵ are as hereinbefore defined, e.g. under conditions known to those skilled in the art (for example: (i) when Lg³ represents a leaving group, reaction at between ambient temperature and reflux in the presence of an appropriate base (e.g. TEA, K₂CO₃) and a suitable solvent (such as DCM, MeCN, DMF or DMSO); and (ii) when Lg³ represents OH, reaction under Mitsunobu conditions (e.g. those described above in respect of the preparation of compounds of formula I (see process alternative (m))).

In another alternative synthesis, compounds of formula XXVII in which R³ represents C₁₋₆ alkyl optionally substituted by one or more F atoms and X represents O may be prepared by reaction of a compound of formula XXXII,

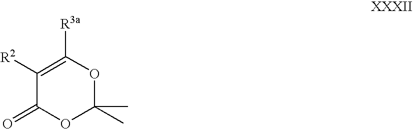

wherein R² and R^{3a} are as hereinbefore defined, with a compound of formula XXXIII,

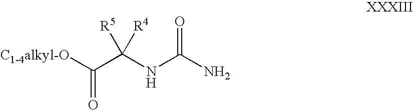

wherein R⁴ and R⁵ are as hereinbefore defined, for example at elevated temperature (such as between 40 and 120° C.), optionally in the presence of a suitable solvent (such as DMF or toluene).

Compounds of formula XXVII in which R³ represents CN may be prepared by reaction of a corresponding compound of formula XXVII in which R³ represents H and R² represents halo (e.g. bromo) with a suitable source of the cyanide anion (e.g. NaCN), for example under conditions known to those skilled in the art (such as reaction at ambient temperature in the presence of a suitable solvent (e.g. DMF)).

Compounds of formula XXVII in which R³ represents C₁₋₆ alkyl substituted by halo and X represents O may be prepared by reaction of a corresponding compound of formula XXVII in which R³ represents C₁₋₆ alkyl substituted by OH and X represents O with a suitable halogenating agent (e.g. the agents described above in relation to the preparation of compounds of formula IV or, when halo is F, diethylaminosulfur trifluoride), for example under conditions known to those skilled in the art.

Compounds of formula XXVII in which R³ represents C₁₋₆ alkyl substituted, on the C-atom that is attached to the pyrimidione ring, by OH and X represents O may be prepared by reaction of a corresponding compound of formula XXVII in which R³ represents C₁₋₆ alkyl and X represents O with a suitable oxidising agent (e.g. selenium dioxide or Na₂S₂O₅), for example under conditions known to those skilled in the art (such as in the presence of a suitable solvent (e.g. dioxane or water)).

Compounds of formula XXVIII may be prepared by reaction of malonic acid with a suitable source of the thiocyanate ion (e.g. potassium thiocyanate) and compounds of formulae XXXIV and XXXV,

{R^{3a}C(O)}₂O XXXIV

R^{3a}C(O)OH XXXV

wherein R^{3a} is as hereinbefore defined, for example under conditions known to those skilled in the art (e.g. by reaction at ambient temperature).

Compounds of formulae III, IX, X, XIII, XIV, XVII, XXIII, XXIV, XXV, XXVII (in which R³ represents H and 2 is halo), XXIX, XXX, XXXI, XXX, XXXIII, XIV, and XXXV are either commercially available, are known in the literature, or may be obtained by analogy with the processes described herein, or by conventional synthetic procedures, in accordance with standard techniques, from readily available starting materials using appropriate reagents and reaction conditions. In this respect, compounds described herein may also be obtained by analogy with synthetic procedures described in the prior art documents mentioned above (and WO 94/20467, WO 94/29336, WO 95/23609, WO 96/06832, WO 96/06849, WO 97/11693, WO 97/24135, WO 98/01422, WO 01/68605, WO 99/26920, WO 01/79155, WO 01/68605, WO 96/18644, WO 97/01338, WO 97/30708, WO 98/16547, WO 99/26926, WO 00/73302, WO 01/04117, WO 01/79262, WO 02/064140, WO 02/057225, WO 03/29224, WO 2005/040137, U.S. Pat. No. 5,668,289, U.S. Pat. No. 5,792,779 and WO 95/35313 in particular).

Substituents on alkyl, alkenyl, cycloalkyl, cycloalkenyl, aryl and heterocyclic groups in compounds of formulae I to XXXV may be introduced and/or interconverted using techniques well known to those skilled in the art by way of standard functional groups interconversions, in accordance with standard techniques, from readily available starting materials using appropriate reagents and reaction conditions. For example, hydroxy may be converted to alkoxy, phenyl may be halogenated to give halophenyl, halo may be displaced by cyano, etc.

The skilled person will also appreciate that various standard substituent or functional group interconversions and transformations within certain compounds of formula I will provide other compounds of formula I. For example, hydroxyamidino may be reduced to amidino.

Compounds of formula I may be isolated from their reaction mixtures using conventional techniques.

In accordance with the present invention, pharmaceutically acceptable derivatives of compounds of formula I also include “protected” derivatives, and/or compounds that act as prodrugs, of compounds of formula I.

Compounds that may act as prodrugs of compounds of formula I that may be mentioned include compounds of formula I in which R^{13a}, R^{13b} or R^{13c} is other than H or R^{14c} represents C(O)O—C₁₋₆ alkyl, the alkyl part of which group is optionally substituted by aryl and/or one or more halo atoms (e.g. compounds in which R^{14c} represents C(O)O-tert-butyl).

The compounds of the invention may exhibit tautomerism. All tautomeric forms and mixtures thereof are included within the scope of the invention. Particular tautomeric forms that may be mentioned include those connected with the position of the double bond in the amidine or guanidine functionalities that the groups R^{a} to R^{d} may represent.

Compounds of the invention may also contain one or more asymmetric carbon atoms and may therefore exhibit optical and/or diastereoisomerism. Diastereoisomers may be separated using conventional techniques, e.g. chromatography. The various stereoisomers may be isolated by separation of a racemic or other mixture of the compounds using conventional, e.g. HPLC techniques. Alternatively the desired optical isomers may be made by reaction of the appropriate optically active starting materials under conditions which will not cause racemisation or epimerisation, or by derivatisation, for example with a homochiral acid followed by separation of the diastereomeric derivatives by conventional means (e.g. HPLC, chromatography over silica). All stereoisomers are included within the scope of the invention.

It will be appreciated by those skilled in the art that in the processes described above and hereinafter the functional groups of intermediate compounds may need to be protected by protecting groups.

Functional groups that it is desirable to protect include hydroxy, amino and carboxylic acid. Suitable protecting groups for hydroxy include optionally substituted and/or unsaturated alkyl groups (e.g. methyl, allyl, benzyl or tert-butyl), trialkylsilyl or diarylalkylsilyl groups (e.g. t-butyldimethylsilyl, t-butyldiphenylsilyl or trimethylsilyl) and tetrahydropyranyl. Suitable protecting groups for carboxylic acid include C₁₋₆ alkyl or benzyl esters. Suitable protecting groups for amino and amidino include t-butyloxycarbonyl, benzyloxycarbonyl or 2-trimethylsilylethoxycarbonyl (Teoc). Amidino nitrogens may also be protected by hydroxy or alkoxy groups, and may be either mono- or diprotected.

The protection and deprotection of functional groups may take place before or after coupling, or before or after any other reaction in the above-mentioned schemes.

Protecting groups may be removed in accordance with techniques that are well known to those skilled in the art and as described hereinafter.

Persons skilled in the art will appreciate that, in order to obtain compounds of the invention in an alternative, and, on some occasions, more convenient, manner, the individual process steps mentioned hereinbefore may be performed in a different order, and/or the individual reactions may be performed at a different stage in the overall route (i.e. substituents may be added to and/or chemical transformations performed upon different intermediates to those mentioned hereinbefore in conjunction with a particular reaction). This may negate, or render necessary, the need for protecting groups.

The type of chemistry involved will dictate the need, and type, of protecting groups as well as the sequence for accomplishing the synthesis.

The use of protecting groups is described in “Protective Groups in Organic Chemistry”, edited by J W F McOmie, Plenum Press (1973), and “Protective Groups in Organic Synthesis”, 3^{rd} edition, T. W. Greene & P. G. M. Wutz, Wiley-Interscience (1999).

Protected derivatives of compounds of the invention may be converted chemically to compounds of the invention using standard deprotection techniques (e.g. hydrogenation). The skilled person will also appreciate that certain compounds of formula I (e.g. compounds in which R^{13a}, R^{13b} or R^{13c} is other than H) may also be referred to as being “protected derivatives” of other compounds of formula I (e.g. those in which R^{13a}, R^{13b} or R^{13c} represents H).

Those skilled in the art will also appreciate that certain compounds of formula I will be useful as intermediates in the synthesis of certain other compounds of formula I.

Some of the intermediates referred to hereinbefore are novel. According to a further aspect of the invention there is thus provided: (a) a compound of formula II, or a protected derivative thereof; (b) a compound of formula IV, or a protected derivative thereof; (c) a compound of formula V, or a protected derivative thereof; (d) a compound of formula VI, or a protected derivative thereof; (e) a compound of formula VII, or a protected derivative thereof; (f) a compound of formula VIII, or a protected derivative thereof; (g) a compound of formula XII, or a protected derivative thereof; (h) a compound of formula XIV, or a protected derivative thereof; (i) a compound of formula XV, or a protected derivative thereof; (j) a compound of formula XVIII, or a protected derivative thereof; (k) a compound of formula XIX, or a protected derivative thereof; (l) a compound of formula XXI, or a protected derivative thereof; (m) a compound of formula XXV, or a protected derivative thereof; and (n) a compound of formula XXVI, or a protected derivative thereof.

### Medical and Pharmaceutical Use

Compounds of the invention may possess pharmacological activity as such. However, other compounds of the invention may not possess such activity, but may be administered parenterally or orally, and may thereafter be metabolised in the body to form compounds that are pharmacologically active. Such compounds (which also includes compounds that may possess some pharmacological activity, but that activity is appreciably lower than that of the “active” compounds to which they are metabolised), may therefore be described as “prodrugs” of the active compounds.

Thus, the compounds of the invention are useful because they possess pharmacological activity, and/or are metabolised in the body following oral or parenteral administration to form compounds which possess pharmacological activity. The compounds of the invention are therefore indicated as pharmaceuticals.

According to a further aspect of the invention there is thus provided the compounds of the invention for use as pharmaceuticals.

In particular, compounds of the invention are potent inhibitors of thrombin either as such and/or (e.g. in the case of prodrugs), are metabolised following administration to form potent inhibitors of thrombin, for example as may be demonstrated in the tests described below.

By “prodrug of a thrombin inhibitor”, we include compounds that form a thrombin inhibitor, in an experimentally-detectable amount, and within a predetermined time (e.g. about 1 hour), following oral or parenteral administration (see, for example, Test E below) or, alternatively, following incubation in the presence of liver microsomes (see, for example, Test F below).

The compounds of the invention are thus expected to be useful in those conditions where inhibition of thrombin is beneficial (as determined by reference to a clinically relevant end-point, e.g. conditions, such as thrombo-embolisms, where inhibition of thrombin is required or desired, and/or conditions where anticoagulant therapy is indicated), including the following:

The treatment and/or prophylaxis of thrombosis and hypercoagulability in blood and/or tissues of animals including man. It is known that hypercoagulability may lead to thrombo-embolic diseases. Conditions associated with hypercoagulability and thrombo-embolic diseases are usually designated as thrombophilia conditions. These conditions include, but are not limited to, inherited or acquired activated protein C resistance, such as the factor V-mutation (factor V Leiden), inherited or acquired deficiencies in antithrombin III, protein C, protein S, heparin cofactor II, and conditions with increased plasma levels of the coagulation factors such as caused by the prothrombin G20210A mutation. Other conditions known to be associated with hypercoagulability and thrombo-embolic disease include circulating antiphospholipid antibodies (Lupus anticoagulant), homocysteinemi, heparin induced thrombocytopenia and defects in fibrinolysis, as well as coagulation syndromes (e.g. disseminated intravascular coagulation (DIC)) and vascular injury in general (e.g. due to trauma or surgery). Furthermore, low physical activity, low cardiac output or high age are known to increase the risk of thrombosis and hypercoagulability may be just one of several factors underlying the increased risk. These conditions include, but are not limited to, prolonged bed rest, prolonged air travelling, hospitalisation for an acute medical disorder such as cardiac insufficiency or respiratory insufficiency. Further conditions with increased risk of thrombosis with hypercoagulability as one component are pregnancy and hormone treatment (e.g. oestrogen).

The treatment of conditions where there is an undesirable excess of thrombin without signs of hypercoagulability, for example in neurodegenerative diseases such as Alzheimer's disease.

Particular disease states which may be mentioned include the therapeutic and/or prophylactic treatment of venous thrombosis (e.g. deep venous thrombosis, DVT) and pulmonary embolism, arterial thrombosis (e.g. in myocardial infarction, unstable angina, thrombosis-based stroke and peripheral arterial thrombosis), and systemic embolism usually from the atrium during atrial fibrillation (e.g. non valvular or valvular atrial fibrillation) or from the left ventricle after transmural myocardial infarction, or caused by congestive heart failure; prophylaxis of re-occlusion (i.e. thrombosis) after thrombolysis, percutaneous trans-luminal angioplasty (PTA) and coronary bypass operations; the prevention of thrombosis after microsurgery and vascular surgery in general.

Further indications include the therapeutic and/or prophylactic treatment of disseminated intravascular coagulation caused by bacteria, multiple trauma, intoxication or any other mechanism; anticoagulant treatment when blood is in contact with foreign surfaces in the body such as vascular grafts, vascular stents, vascular catheters, mechanical and biological prosthetic valves or any other medical device; and anticoagulant treatment when blood is in contact with medical devices outside the body such as during cardiovascular surgery using a heart-lung machine or in haemodialysis; the therapeutic and/or prophylactic treatment of idiopathic and adult respiratory distress syndrome, pulmonary fibrosis following treatment with radiation or chemotherapy, chronic obstructive lung disease, septic shock, septicemia, inflammatory responses, which include, but are not limited to, edema, acute or chronic atherosclerosis such as coronary arterial disease and the formation of atherosclerotic plaques, cardiac insufficiency, cerebral arterial disease, cerebral infarction, cerebral thrombosis, cerebral embolism, peripheral arterial disease, ischaemia, angina (including unstable angina), reperfusion damage, restenosis after percutaneous trans-luminal angioplasty (PTA) and coronary artery bypass surgery.

Compounds of the invention that inhibit trypsin and/or thrombin may also be useful in the treatment of pancreatitis.

The compounds of the invention are thus indicated both in the therapeutic and/or prophylactic treatment of these conditions.

According to a further aspect of the present invention, there is provided a method of treatment of a condition where inhibition of thrombin is required which method comprises administration of a therapeutically effective amount of a compound of the invention to a person suffering from, or susceptible to, such a condition.

The compounds of the invention will normally be administered orally, intravenously, subcutaneously, buccally, rectally, dermally, nasally, tracheally, bronchially, by any other parenteral route or via inhalation, in the form of pharmaceutical preparations comprising compound of the invention either as a free base, or a pharmaceutically acceptable nontoxic organic or inorganic acid addition salt, in a pharmaceutically acceptable dosage form.

Preferred route of administration of compounds of the invention are oral.

Depending upon the disorder and patient to be treated and the route of administration, the compositions may be administered at varying doses.

The compounds of the invention may also be combined and/or co-administered with any antithrombotic agent(s) with a different mechanism of action, such as one or more of the following: the anticoagulants unfractionated heparin, low molecular weight heparin, other heparin derivatives, synthetic heparin derivatives (e.g. fondaparinux), vitamin K antagonists, synthetic or biotechnological inhibitors of other coagulation factors than thrombin (e.g. synthetic FXa, FVIIa and FIXa inhibitors, and rNAPc2), the antiplatelet agents acetylsalicylic acid, ticlopidine and clopidogrel; thromboxane receptor and/or synthetase inhibitors; fibrinogen receptor antagonists; prostacyclin mimetics; phosphodiesterase inhibitors; ADP-receptor (P2X₁, P2Y₁, P2Y₁₂ [P₂T]) antagonists; and inhibitors of carboxypeptidase U (CPU or TAFIa) and inhibitors of plasminogen activator inhibitor-1 (PAI-1).

The compounds of the invention may further be combined and/or co-administered with thrombolytics such as one or more of tissue plasminogen activator (natural, recombinant or modified), streptokinase, urokinase, prourokinase, anisoylated plasminogenstreptokinase activator complex (APSAC), animal salivary gland plasminogen activators, and the like, in the treatment of thrombotic diseases, in particular myocardial infarction.

According to a further aspect of the invention there is thus provided a pharmaceutical formulation including a compound of the invention, in admixture with a pharmaceutically acceptable adjuvant, diluent or carrier.

Suitable daily doses of the compounds of the invention in therapeutic treatment of humans are about 0.001-100 mg/kg body weight at peroral administration and 0.001-50 mg/kg body weight at parenteral administration.

For the avoidance of doubt, as used herein, the term “treatment” includes therapeutic and/or prophylactic treatment.

Compounds of the invention have the advantage that they may be more efficacious, be less toxic, be longer acting, have a broader range of activity, be more selective (e.g. for inhibiting thrombin over other serine proteases, in particular trypsin and those involved in haemostasis), be more potent, produce fewer side effects, be more easily absorbed, and/or have a better pharmacokinetic profile (e.g. higher oral bioavailability and/or lower clearance), than, and/or have other useful pharmacological, physical, or chemical, properties over, compounds known in the prior art.

### Biological Tests

The following test procedures may be employed.

### Test A

### Determination of Thrombin Clotting Time (TT)

The inhibitor solution (25 μL) is incubated with plasma (25 μL) for three minutes. Human thrombin (T 6769; Sigma Chem. Co or Hematologic Technologies) in buffer solution, pH 7.4 (25 μL, 4.0 NIH units/mL), is then added and the clotting time measured in an automatic device (KC 10; Amelung).

The thrombin clotting time (TT) is expressed as absolute values (seconds) as well as the ratio of TT without inhibitor (TT₀) to TT with inhibitor (TTᵢ). The latter ratios (range 1-0) are plotted against the concentration of inhibitor (log transformed) and fitted to sigmoidal dose-response curves according to the equation

y=a/[1+(x/IC₅₀)^{s}]

where: a=maximum range, i.e. 1; s=slope of the dose-response curve; and IC₅₀=the concentration of inhibitor that doubles the clotting time. The calculations are processed on a PC using the software program GraFit Version 3, setting equation equal to: Start at 0, define end=1 (Erithacus Software, Robin Leatherbarrow, Imperial College of Science, London, UK).

### Test B

Determination of Thrombin Inhibition with a Chromogenic, Robotic Assay

The thrombin inhibitor potency is measured with a chromogenic substrate method, in a Plato 3300 robotic microplate processor (Rosys AG, CH-8634 Hombrechtikon, Switzerland), using 96-well, half volume microtitre plates (Costar, Cambridge, Mass., USA; Cat No 3690). Stock solutions of test substance in DMSO (72 μL), 0.1-1 mmol/L, are diluted serially 1:3 (24+48 μL) with DMSO to obtain ten different concentrations, which are analysed as samples in the assay. 2 μL of test sample is diluted with 124 μL assay buffer, 12 μL of chromogenic substrate solution (S-2366, Chromogenix, Mölndal, Sweden) in assay buffer and finally 12 μL of a-thrombin solution (Human a-thrombin, Sigma Chemical Co. or Hematologic Technologies) in assay buffer, are added, and the samples mixed. The final assay concentrations are: test substance 0.00068-133 μmol/L, S-2366 0.30 mmol/L, a-thrombin 0.020 NIHU/mL. The linear absorbance increment during 40 minutes incubation at 37° C. is used for calculation of percentage inhibition for the test samples, as compared to blanks without inhibitor. The IC₅₀-robotic value, corresponding to the inhibitor concentration which causes 50% inhibition of the thrombin activity, is calculated from a log concentration vs. % inhibition curve.

### Test C

### Determination of the Inhibition Constant Kᵢ for Human Thrombin

Kᵢ-determinations are made using a chromogenic substrate method, performed at 37° C. on a Cobas Bio centrifugal analyser (Roche, Basel, Switzerland). Residual enzyme activity after incubation of human 1-thrombin with various concentrations of test compound is determined at three different substrate concentrations, and is measured as the change in optical absorbance at 405 nm.

Test compound solutions (100 μL; normally in buffer or saline containing BSA 10 g/L) are mixed with 200 μL of human a-thrombin (Sigma Chemical Co) in assay buffer (0.05 mol/L Tris-HCl pH 7.4, ionic strength 0.15 adjusted with NaCl) containing BSA (10 g/L), and analysed as samples in the Cobas Bio. A 60 μL sample, together with 20 μL of water, is added to 320 μL of the substrate S-2238 (Chromogenix AB, Mölndal, Sweden) in assay buffer, and the absorbance change (?A/min) is monitored. The final concentrations of S-2238 are 16, 24 and 50 μmol/L and of thrombin 0.125 NIH U/mL.

The steady state reaction rate is used to construct Dixon plots, i.e. diagrams of inhibitor concentration vs. 1/(?A/min). For reversible, competitive inhibitors, the data points for the different substrate concentrations typically form straight lines which intercept at x=−Kᵢ.

### Test D

### Determination of Activated Partial Thromboplastin Time (APTT)

APTT is determined in pooled normal human citrated plasma with the reagent PTT Automated 5 manufactured by Stago. The inhibitors are added to the plasma (10 μL inhibitor solution to 90 μL plasma) and incubated with the APTT reagent for 3 minutes followed by the addition of 100 μL of calcium chloride solution (0.025 M) and APTT is determined by use of the coagulation analyser KC10 (Amelung) according to the instructions of the reagent producer.

The clotting time is expressed as absolute values (seconds) as well as the ratio of APTT without inhibitor (APTT₀) to APTT with inhibitor (APTTᵢ). The latter ratios (range 1-0) are plotted against the concentration of inhibitor (log transformed) and fitted to sigmoidal dose-response curves according to the equation

y=a/[1+(x/IC₅₀)^{s}]

where: a=maximum range, i.e. 1; s=slope of the dose-response curve; and IC₅₀=the concentration of inhibitor that doubles the clotting time. The calculations are processed on a PC using the software program GraFit Version 3, setting equation equal to: Start at 0, define end=1 (Erithacus Software, Robin Leatherbarrow, Imperial College of Science, London, UK).

IC₅₀APTT is defined as the concentration of inhibitor in human plasma that doubled the Activated Partial Thromboplastin Time.

### Test E

### Determination of Plasma Clearance and Oral Bioavailability in Rat

Plasma clearance and oral bioavailability are estimated in female Sprague Dawley rats. The compound is dissolved in water or another appropriate vehicle. For determination of plasma clearance the compound is administered as a subcutaneous (sc) or an intravenous (iv) bolus injection at a dose of 1-4 μmol/kg. Blood samples are collected at frequent intervals up to 24 hours after drug administration. For bioavailability estimates, the compound is administered orally at, 10 μmol/kg via gavage and blood samples are collected frequently up to 24 hours after dosing. The blood samples are collected in heparinized tubes and centrifuged within 30 minutes, in order to separate the plasma from the blood cells. The plasma is transferred to plastic vials with screw caps and stored at −20° C. until analysis. Prior to the analysis, the plasma is thawed and 50 μL of plasma samples are precipitated with 150 μL of cold acetonitrile. The samples are centrifuged for 20 minutes at 4000 rpm. 75 μL of the supernatant is diluted with 75 μL of 0.2% formic acid. 10 μL volumes of the resulting solutions are analysed by LC-MS/S and the concentrations of thrombin inhibitor are determined using standard curves. All pharmacokinetic calculations are performed with the computer program WinNonlin™ Professional (Pharsight Corporation, California, USA), or an equivalent program. Area under the plasma concentration-time profiles (AUC) is estimated using the log/linear trapezoidal rule and extrapolated to infinite time. Plasma clearance (CL) of the compound is then determined as

CL=Dose(iv/sc)/AUC(iv/sc).

The oral bioavailability is calculated as

F=CL×AUC(po)/Dose(po).

Plasma clearance is reported as mL/min/kg and oral bioavailability as percentage (%).

### Test F

### Determination of In Vitro (Liver Microsome) Stability

Liver microsomes are prepared from Sprague-Dawley rats and human liver samples according to internal SOPs. The compounds are incubated at 37° C. at a total microsome protein concentration of 0.5 mg/mL in a 0.1 mol/l potassium phosphate buffer at pH 7.4, in the presence of the cofactor, NADPH (1.0 μmol/L). The initial concentration of compound is 1.0 μmol/L. Samples are taken for analysis at 5 time points, 0, 7, 15, 20 and 30 minutes after the start of the incubation. The enzymatic activity in the collected sample is immediately stopped by adding an equal volume of acetonitrile containing 0.8% formic acid. The concentration of compound remaining in each of the collected samples is determined by means of LC-MS/MS. The elimination rate constant (k) of the thrombin inhibitor is calculated as the slope of the plot of ln [Thrombin inhibitor] against incubation time (minutes). The elimination rate constant is then used to calculate the half-life (T_{1/2}) of the thrombin inhibitor, which is subsequently used to calculate the intrinsic clearance (CLint) of the thrombin inhibitor in liver microsomes as:

$$CLint ( in μ L / min / mg ) = \frac{( l n 2 \times incubation volume )}{( {T}_{1 / 2} \times protein concentration )}$$

### Test G

### Venous Thrombosis Model

The thrombogenic stimuli are vessel damage and blood flow stasis. Rats are anaesthetised and the abdomen is opened. A partial occlusion on the caval vein, caudal to the left kidney-vein, is obtained with a snare around the vein and a cannula, which is than removed. A filter-paper soaked with FeCl₃ is placed on the external surface of the distal part of the caval vein. The abdomen is filled with saline and closed. At the end of the experiment the rat is sacrificed, the caval vein is extirpated, the thrombus harvested and its wet weight determined.

### EXAMPLES

### General Experimental Details

High resolution mass spectra were recorded on a Micromass LCT mass spectrometer equipped with an electrospray interface (LC-HRMS). ¹H NMR measurements were performed on Varian UNITY plus 400, 500 and 600 spectrometers, operating at ¹H frequencies of 400, 500 and 600 MHz respectively. Chemical shifts are given in ppm with the solvent as internal standard. Flash chromatography separations were performed using Merck Silica gel 60 (0.063-0.200 mm). The compounds named below were named using ACD/name version 8.05/13 Apr. 2004 available from Advanced Chemistry Development Inc., Canada.

### Reagents

The following lists of reagents were used in the Preparations and Examples below. Unless otherwise stated, each of these reagents is commercially available.

### List 1

(a) 2-Chloro-5-fluorobenzaldehyde.
(b) 3,5-Dimethylisoxazole-4-carbaldehyde.
(c) 5-Chloro-1,3-dimethyl-1H-pyrazole-4-carbaldehyde.

### List 2

(a) (2-Aminomethyl-4-chlorobenzyl)carbamic acid tert-butyl ester (obtainable as described in WO 02/050056).
(b) tert-Butyl [5-(aminomethyl)-4,6-dimethylpyridin-2-yl]carbamate (obtainable as described in WO 97/01338).
(c) [5-Chloro-2-(1H-tetrazol-1-yl)benzyl]amine (obtainable as described in WO 02/064559).
(d) 2-[2-(Aminomethyl)-4-chlorophenoxy]-N-ethylacetamide (obtainable as described in WO 97/30708).
(e) tert-Butyl [2-(aminomethyl)benzyl]carbamate (obtainable as described in WO 02/057225).

### Preparation of Intermediates

### Preparation 1

### tert-Butyl (3-amino-6-methyl-2,4-dioxo-3,4-dihydropyrimidin-1(2H)-yl)acetate

### (a) 5-Acetyl-4-hydroxy-2H-1,3-thiazine-2,6(3H)-dione

To a suspension of malonic acid (52.0 g, 0.5 mol) and potassium thiocyanate (48.6 g, 0.5 mol) in acetic acid (250 mL) was added acetic anhydride (102 g, 1.0 mol). The resulting yellow solution was stirred at rt for 24 h, giving a thick light yellow precipitate in dark solution. The mixture was diluted with water, and extracted with DCM/MeOH (9:1). The combined organic phases were dried, filtered and concentrated. The residue was suspended in diethyl ether, filtered, washed with diethyl ether and dried to give the product as a light yellow solid (43 g, 46%). This material was used directly in the next step without further purification.

### (b) tert-Butyl (6-methyl-2,4-dioxo-3,4-dihydropydrimidin-1(2H)-yl)acetate

A solution of 5-acetyl-4-hydroxy-2H-1,3-thiazine-2,6(3H)-dione (6.55 g, 35 mmol; see step (a) above) and glycine tert-butyl ester hydrochloride salt (8.80 g, 52.5 mmol) in pyridine (100 mL) was heated at reflux overnight. The mixture was concentrated and the residue was purified (flash chromatography, DCM/EtOAc, 9:1 to 1:1) to give the product as solid. The solid was suspended in diethyl ether/heptane (1:1), filtered and washed with the same solvent mixture to give tert-butyl (6-methyl-2,4-dioxo-3,4-dihydropyrimidin-1(2H)-yl)acetate as a colourless solid (3.50 g, 42%).

### (c) tert-Butyl (3-amino-6-methyl-2,4-dioxo-3,4-dihydropyrimidin-1(2H)-yl)-acetate

To a suspension of NaH (572 mg, 60% in mineral oil, 14.3 mmol) in DMF (10 mL) was added a solution of tert-butyl (6-methyl-2,4-dioxo-3,4-dihydropyrimidin-1(2H)-yl)acetate (3.12 g, 13.0 mmol; see step (b) above) in DMF (30 mL). After ca. 30 min, a solution of O-(2,4-dinitrophenyl)-hydroxylamine (2.85 g, 14.3 mmol) in DMF (30 mL) was added. The mixture was concentrated and the residue was suspended in NaOH (aq. 0.5 M) and extracted with DCM. The combined organic phases were dried, filtered and concentrated. Purification (flash chromatography, DCM/EtOAc, 1:1 to 0:1) gave an oil that solidified on standing. This material was suspended in diethyl ether, the solid was filtered off, washed with diethyl ether and dried to give the title compound as a colourless solid (1.71 g, 52%).

¹H NMR (500 MHz, CDCl₃) d 5.71 (s, 1H), 5.14 (bs, 2H), 4.54 (s, 2H), 2.17 (s, 3H), 1.46 (s, 9H)

### Preparation 2

### tert-Butyl [3-[(2,2-difluoro-2-pyridin-2-ylethyl)amino]-6-methyl-2,4-dioxo-3,4-dihydropyridin-1(2H)-yl]acetate

2,6-Di-tert-butyl-4-methylpyridine (148 mg, 0.72 mmol) was added to a solution of 2,2-difluoro-2-pyridin-2-ylethyl trifluoromethanesulfonate (140 mg, 0.48 mmol; prepared according to the method described in Organic Process & Development, 2004, 8 (2), 192-200 and tert-butyl (3-amino-6-methyl-2,4-dioxo-3,4-dihydropyrimidin-1(2H)-yl)acetate (80 mg, 0.31 mmol) in 1,2-dichloroethane (4 mL). The mixture was heated in a microwave oven at 120° C. for 20 min and was then concentrated. Purification (flash chromatography (heptane/EtOAc, 3:7 to 0:1) gave 153 mg (80.3%) of the title compound.

### Preparation 3

### tert-Butyl [3-[(2-chloro-5-fluorobenzyl)amino]-6-methyl-2,4-dioxo-3,4-dihydro-pyrimidin-1(2H)-yl]acetate

### (a) tert-Butyl [3-{[(1E)-(2-chloro-5-fluorophenyl)methylene]amino}-6-methyl-2,4-dioxo-3,4-dihydropyrimidin-1(2H)-yl]acetate

A solution of 2-chloro-5-fluorobenzaldehyde (250 mg, 0.98 mmol) and tert-butyl (3-amino-6-methyl-2,4-dioxo-3,4-dihydropyrimidin-1 (2H)-yl)acetate (186 mg, 1.18 mmol; see Preparation 1 above) in MeOH (10 mL) and HOAc (2 mL) was stirred overnight at 40° C. under nitrogen. The reaction mixture was concentrated and purified by flash chromatography (heptane/EtOAc, 1:1) to give 356 mg (91.8%) of the sub-title compound.

### (b) tert-Butyl [3-[(2-chloro-5-fluorobenzyl)amino]-6-methyl-2,4-dioxo-3,4-dihydropyrimidin-1(2H)-yl]acetate

Sodium cyanoborohydride (142.9 mg, 2.27 mmol) was added to a solution of tert-butyl [3-{[(1E)-(2-chloro-5-fluorophenyl)methylene]amino}-6-methyl-2,4-dioxo-3,4-dihydropyrimidin-1(2H)-yl]acetate (300 mg, 0.75792 mmol; see step (a) above) in AcOH (2 mL) and MeOH (6 mL) and the mixture was stirred at rt overnight. The reaction mixture was concentrated, diluted with dichloromethane and washed with saturated aqueous NaHCO₃. The organic phase was filtered through a phase separator and concentrated to give 297 mg (98.5%) of the title compound.

### Preparation 4

### [3-[(2,2-Difluoro-2-pyridin-2-ylethyl)amino]-6-methyl-2,4-dioxo-3,4-dihydropyrimidin-1(2H)-yl]acetic acid

A solution of tert-butyl [3-[(2,2-difluoro-2-pyridin-2-ylethyl)amino]-6-methyl-2,4-dioxo-3,4-dihydropyrimidin-1(2H)-yl]acetate (153 mg, 0.386 mmol; see Preparation 2 above) in TFA (3 mL) was stirred at rt for 3 h and was then concentrated. The residue was dissolved in EtOAc (5 mL, saturated with HCl (g)) and the mixture was stirred for 20 min. Concentration gave the title compound as the hydrochloride salt (125 mg, 86%).

### Preparation 5

### [3-[(2-chloro-5-fluorobenzyl)amino]-6-methyl-2,4-dioxo-3,4-dihydro-pyrimidin-1(2H)-yl]acetic acid

The title compound was prepared according to a procedure analogous to that described in Preparation 4 above, using tert-butyl [3-[(2-chloro-5-fluorobenzyl)amino]-6-methyl-2,4-dioxo-3,4-dihydro-pyrimidin-1(2H)-yl]acetate (see Preparation 3 above) in place of tert-butyl [3-[(2,2-difluoro-2-pyridin-2-ylethyl)amino]-6-methyl-2,4-dioxo-3,4-dihydropyrimidin-1(2H)-yl]acetate.

### Preparation 6

Using procedures analogous to those described in Preparations 3, 4 and 7 and, in the reaction equivalent to step (a) of Preparation 3, employing the appropriate aldehyde from List 1 above in place of 2-chloro-5-fluorobenzaldehyde, the following compounds were prepared.
(a) {[(3,5-Dimethylisoxazol-4-yl)methyl]amino}-6-methyl-2,4-dioxo-3,4-dihydro-pyrimidin-1(2H)-yl]acetic acid.
(b) {[(5-chloro-1,3-dimethyl-1H-pyrazol-4-yl)methyl]amino}-6-methyl-2,4-dioxo-3,4-dihydro-pyrimidin-1(2H)-yl]acetic acid.
(c) 2-[3-[(2-chloro-5-fluoro-phenyl)methylamino]-6-methyl-2-oxo-4-thioxo-pyrimidin-1-yl]acetic acid.

### Preparation 7

### tert-butyl 2-[3-[(2-chloro-5-fluoro-phenyl)methylamino]-6-methyl-2-oxo-4-thioxo-pyrimidin-1-yl]acetate

tert-butyl 2-[3-[(2-chloro-5-fluoro-phenyl)methylamino]-6-methyl-2,4-dioxo-pyrimidin-1-yl]acetate (127 mg, 0.26 mmol, see Preparation 3 above) was added to pyridine (3 mL). Lawesson's reagent was added (120 mg, 0.30 mmol). The reaction was heated to reflux overnight. The pyridine was removed by evaporation. Flash chromatography of the crude (Toluene/Acetone gradient 20:1 to 1:1, followed by addition of MeOH) yielded 35 mg of the title compound.

### Synthesis of Compounds of Formula I

### Example 1

### tert-Butyl {4-chloro-2-[({[3-[(2,2-difluoro-2-pyridin-2-ylethyl)amino]-6-methyl-2,4-dioxo-3,4-dihydropyrimidin-1(2H)-yl]acetyl}amino)methyl]benzyl}carbamate

A solution of [3-[(2,2-difluoro-2-pyridin-2-ylethyl)amino]-6-methyl-2,4-dioxo-3,4-dihydropyrimidin-1(2H)-yl]acetic acid hydrochloride (50 mg, 0.147 mmol; see Preparation 4 above), (2-aminomethyl-4-chlorobenzyl)carbamic acid tert-butyl ester (59.7 mg, 0.22 mmol; see List 2 above), HOAt (40 mg, 0.29 mmol), EDC (84.5 mg, 0.44 mmol) and triethylamine (123 μL, 0.88 mmol) in DMF (2 mL) was stirred at rt for 72 h. The resulting crude product was purified by HPLC (C8 column, 20×2500 mm, 15 mL/min, MeCN/water and 0.1 M ammonium acetate, gradient 5%-60% MeCN). Lyophilization then gave 72 mg (82.6%) of the title compound.

¹H NMR (400 MHz, CDCl₃): d 8.60 (d, 1H), 7.79 (t, 1H), 7.71 (br s, 1H), 7.68 (d, 1H), 7.35 (t, 1H), 7.24 (s, 1H), 7.22 (s, 1H), 6.18 (s, 1H), 6.11 (t, 1H), 5.57 (s, 1H), 5.34 (t, 1H), 4.46 (s, 2H), 4.40 (d, 2H), 4.24 (d, 2H), 3.86 (dt, 2H), 2.19 (s, 3H), 1.39 (s, 9H)

### Example 2

### N-[2-(Aminomethyl)-5-chlorobenzyl]-2-[3-[(2,2-difluoro-2-pyridin-2-ylethyl)-amino]-6-methyl-2,4-dioxo-3,4-dihydropyrimidin-1(2H)-yl]acetamide

tert-Butyl {4-chloro-2-[({[3-[(2,2-difluoro-2-pyridin-2-ylethyl)amino]-6-methyl-2,4-dioxo-3,4-dihydropyrimidin-1(2H)-yl]acetyl}amino)methyl]benzyl}carbamate (72 mg, 0.121 mmol; see Example 1 above) was dissolved in EtOAc (saturated with HCl (g)) and the mixture was stirred at rt overnight. The reaction mixture was concentrated to give 62 mg (96.5%) of the hydrochloride salt of the title compound.

¹H NMR (400 MHz, DMSO): d 8.86 (t, 1H), 8.55 (d, 1H), 7.89 (t, 1H), 7.65 (d, 1H), 7.50-7.30 (m, 4H), 5.55 (s, 1H), 4.43 (s, 2H), 4.34 (d, 2H), 4.02 (d, 2H), 3.70 (t, 2H), 2.05 (s, 3H)

HRMS (ESI) calculated for C₂₂H₂₄N₆O₃F₂Cl 493.1566 (M+H)⁺. found 493.1559.

### Example 3

Using procedures analogous to those set out in Example 1 above, employing an acid reagent from one of Preparations 4 to 6 above and an appropriate amine reagent from List 2 above, the following compounds were prepared.
(a) N-[5-Chloro-2-(1H-tetrazol-1-yl)benzyl]-2-[3-[(2,2-difluoro-2-pyridin-2-ylethyl)amino]-6-methyl-2,4-dioxo-3,4-dihydropyrimidin-1(2H)-yl]acetamide.

¹H NMR (400 MHz, DMSO-d₆): d 9.79 (s, 1H), 8.70 (t, 1H), 8.57 (d, 1H), 7.91 (t, 1H), 7.68 (d, 1H), 7.59 (s, 3H), 7.48 (t, 1H), 6.16 (t, 1H), 5.57 (s, 1H), 4.38 (s, 2H), 4.12 (d, 2H), 3.79-3.65 (m, 2H), 2.05 (s, 3H)

HRMS (ESI) calculated for C₂₂H₂₁N₉O₃ClF₂ 532.1424 (M+H)⁺. found 532.4136.
(b) 2-{4-Chloro-2-[({[3-[(2,2-difluoro-2-pyridin-2-ylethyl)amino]-6-methyl-2,4-dioxo-3,4-dihydropyrimidin-1(2H)-yl]acetyl}amino)methyl]phenoxy}-N-ethyl-acetamide.

¹H NMR (400 MHz, CDCl₃): d 8.58 (d, 1H), 7.79 (t, 1H), 7.66 (s, 1H), 7.64 (s, 1H), 7.43 (t, 1H), 7.35 (t, 1H), 7.26-7.14 (m, 2H), 6.94 (t, 1H), 6.71 (d, 1H), 6.14 (t, 1H), 5.57 (s, 1H), 4.51-4.36 (m, 6H), 3.83 (dt, 2H), 3.32 (q, 2H), 2.21 (s, 3H), 1.14 (t, 3H)

HRMS (ESI) calculated for C₂₅H₂₈N₆O₅ClF₂ 565.1778 (M+H)⁺. found 565.1771.
(c) 2-{4-Chloro-2-[({[3-[(2-chloro-5-fluorobenzyl)amino]-6-methyl-2,4-dioxo-3,4-dihydropyrimidin-1(2H)-yl]acetyl}amino)methyl]phenoxy}-N-ethyl-acetamide.

¹H NMR (400 MHz, CDCl₃): d 7.40 (t, 1H), 7.31-7.15 (m, 3H), 7.10 (dd, 1H), 6.96-6.83 (m, 2H), 6.72 (d, 1H), 5.99 (t, 1H), 5.60 (s, 1H), 4.47 (d, 2H), 4.42 (s, 4H), 4.14 (d, 2H), 3.32 (qv, 2H), 2.22 (s, 3H), 1.14 (t, 3H)

HRMS (ESI) calculated for C₂₅H₂₇N₅O₅Cl₂F 566.1373 (M+H)⁺. found 566.1368.
(d) 2-[3-[(2-Chloro-5-fluorobenzyl)amino]-6-methyl-2,4-dioxo-3,4-dihydro-pyrimidin-1(2H)-yl]-N-[5-chloro-2-(1H-tetrazol-1-yl)benzyl]acetamide.

¹H NMR (400 MHz, CD₃OD): d 8.95 (s, 1H), 7.63 (d, 1H), 7.45 (dd, 1H), 7.30-7.24 (m, 2H), 7.11 (dd, 1H), 6.90 (dt, 1H), 6.82 (t, 1H), 6.03 (t, 1H), 5.62 (s, 1H), 4.42 (s, 2H), 4.21 (dd, 4H), 2.22 (s, 3H)

HRMS (ESI) calculated for C₂₂H₂₀N₈O₃Cl₂F 533.1019 (M+H)⁺. found 533.1029.
(e) 2-{4-Chloro-2-[({[3-{[(3,5-dimethylisoxazol-4-yl)methyl]amino}-6-methyl-2,4-dioxo-3,4-dihydropyrimidin-1(2H)-yl]acetyl}amino)methyl]phenoxy}-N-ethylacetamide.

¹H NMR (500 MHz, DMSO-d₆): d 8.73 (t, 1H), 8.02 (t, 1H), 7.31-7.28 (m, 2H), 6.96 (d, 1H), 5.94 (t, 1H), 5.66 (s, 1H), 4.55 (s, 2H), 4.50 (s, 2H), 4.39 (d, 2H), 3.78 (d, 2H), 3.15 (q, 2H), 2.23 (s, 3H), 2.22 (s, 3H), 2.15 (s, 3H), 1.02 (t, 3H).

HRMS (ESI) calculated for C₂₄H₂₉ClN₆O₆ 533.1915 (M+H)⁺. found 533.1909.
(f) N-[5-Chloro-2-(1H-tetrazol-1-yl)benzyl]-2-[3-{[(3,5-dimethylisoxazol-4-yl)-methyl]amino}-6-methyl-2,4-dioxo-3,4-dihydropyrimidin-1(2H)-yl]acetamide.

¹H NMR (500 MHz, DMSO-d₆): d 9.84 (s, 1H), 8.76 (t, 1H), 7.67-7.64 (m, 3H), 5.94 (t, 1H), 5.64 (s, 1H), 4.48 (s, 2H), 4.18 (d, 2H), 3.78 (d, 2H), 2.22 (s, 3H), 2.21 (s, 3H), 2.12 (s, 3H).

HRMS (ESI) calculated for C₂₁H₂₂ClN₉O₄ 500.1562 (M+H)⁺. found 500.1559.
(g) 2-{4-Chloro-2-[({[3-{[(5-chloro-1,3-dimethyl-1H-pyrazol-4-yl)methyl]-amino}-6-methyl-2,4-dioxo-3,4-dihydropyrimidin-1(2H)-yl]acetyl}amino)-methyl]phenoxy}-N-ethylacetamide.

¹H NMR (400 MHz, DMSO-d₆): d 7.25 (s, 1H), 7.24 (s, 1H), 6.91 (d, 1H), 5.70 (t, 1H), 5.62 (s, 1H), 4.51 (s, 2H), 4.45 (s, 1H), 4.35 (s, 2H), 3.74 (d, 2H), 3.61 (s 3H), 3.27 (s 2H), 3.24 (d, 1H), 3.07 (m, 2H), 2.11 (s, 6H), 0.98 (t, 3H)
(h) 2-[3-{[(5-Chloro-1,3-dimethyl-1H-pyrazol-4-yl)methyl]amino}-6-methyl-2,4-dioxo-3,4-dihydropyrimidin-1(2H)-yl]-N-[5-chloro-2-(1H-tetrazol-1-yl)benzyl]-acetamide.

¹H NMR (400 MHz, CD₃OD): d 9.47 (s, 1H), 7.64 (s 1H), 7.51-7.42 (dd, 2H), 5.59 (s, 1H), 4.47 (s, 2H), 4.21 (s, 2H), 3.87 (s, 2H), 3.63 (s, 3H), 2.16 (s, 3H), 2.14 (d 3H)
(i) 2-{3-[(5-Chloro-1,3-dimethyl-1H-pyrazol-4-ylmethyl)-amino]-6-methyl-2,4-dioxo-3,4-dihydro-2H-pyrimidin-1-yl}-N-(5-chloro-2-tetrazol-1-yl-benzyl)-acetamide.

¹H NMR (400 MHz, CD₃OD): d 9.47 (s, 1H), 7.64 (s 1H), 7.51-7.42 (dd, 2H), 5.59 (s, 1H), 4.47 (s, 2H), 4.21 (s, 2H), 3.87 (s, 2H), 3.63 (s, 3H), 2.16 (s, 3H), 2.14 (d 3H)

HRMS (ESI) calculated for C₂₁H₂₂Cl₂N₁₀O₃ 533.1332 (M+H)⁺. found 533.1302.
(j) 2-[3-[(2-chloro-5-fluoro-phenyl)methylamino]-6-methyl-2-oxo-4-thioxo-pyrimidin-1-yl]-N-[[5-chloro-2-(tetrazol-1-yl)phenyl]methyl]acetamide.

¹H NMR (400 MHz, CD₃OD): d 9.37 (s, 1H), 7.61 (d, 1H), 7.43 (dd, 1H), 7.34 (d, 1H), 7.26 (dd, 1H), 7.18 (dd, 1H) 6.95-6.86 (m, 1H), 6.46 (s, 1H), 4.49 (s, 2H), 4.23 (s, 2H), 4.19 (s, 2H), 2.12 (s, 3H). HRMS (ESI) calculated for C₂₂H₂₀Cl₂F N₈O₂S 549.0791 (M+H)⁺. found 549.0804.

### Example 4

Using procedures analogous to that set out in Example 1 above, and employing an acid reagent from one of Preparations 4 to 6 above and an appropriate amine reagent from List 2 above, the following compounds were prepared.
(a) tert-Butyl {5-[({[3-[(2,2-difluoro-2-pyridin-2-ylethyl)amino]-6-methyl-2,4-dioxo-3,4-dihydropyrimidin-1(2H)-yl]acetyl}amino)methyl]-4,6-dimethylpyridin-2-yl}carbamate.
(b) tert-Butyl {5-[({[3-[(2-chloro-5-fluorobenzyl)amino]-6-methyl-2,4-dioxo-3,4-dihydropyrimidin-1(2H)-yl]acetyl}amino)methyl]-4,6-dimethylpyridin-2-yl}carbamate.

¹H NMR (400 MHz, CDCl₃): d 7.55 (dd, 1H), 7.27 (dd, 1H), 7.24 (s, 1H), 7.16 (s, 1H), 7.13 (dd, 1H), 6.91 (dt, 1H), 6.25 (t, 1H), 5.93 (t, 1H), 5.59 (s, 1H), 4.45-4.34 (m, 4H), 4.13 (d, 2H), 2.39 (s, 3H), 2.29 (s, 3H), 2.26 (s, 3H), 1.48 (s, 9H)
(c) tert-Butyl {2-[({[3-[(2-chloro-5-fluorobenzyl)amino]-6-methyl-2,4-dioxo-3,4-dihydropyrimidin-1(2H)-yl]acetyl}amino)methyl]benzyl}carbamate.

¹H NMR (400 MHz, CDCl₃): d 7.69 (s, 1H), 7.31-7.11 (m, 6H), 6.89 (dt, 1H), 5.98 (s, 1H), 5.57 (s, 1H), 5.32 (t, 1H), 4.50-4.40 (m, 4H), 4.27 (d, 2H), 4.16 (s, 2H), 2.16 (s, 3H), 1.39 (s, 9H)
(d) tert-Butyl {4-chloro-2-[({[3-[(2-chloro-5-fluorobenzyl)amino]-6-methyl-2,4-dioxo-3,4-dihydropyrimidin-1(2H)-yl]acetyl}amino)methyl]benzyl}carbamate.

¹H NMR (400 MHz, CDCl₃): d 7.74 (s, 1H), 7.31-7.10 (m, 5H), 6.89 (dt, 1H), 6.00 (s, 1H), 5.57 (s, 1H), 5.37 (t, 1H), 4.44 (s, 2H), 4.39 (d, 2H), 4.23 (d, 2H) 4.17 (s, 2H), 2.18 (s, 3H), 1.39 (s, 9H)
(e) tert-Butyl {5-[({[3-{[(3,5-dimethylisoxazol-4-yl)methyl]amino}-6-methyl-2,4-dioxo-3,4-dihydropyrimidin-1(2H)-yl]acetyl}amino)methyl]-4,6-dimethylpyridin-2-yl}carbamate.
(f) tert-Butyl {4-chloro-2-[({[3-{[(3,5-dimethylisoxazol-4-yl)methyl]amino}-6-methyl-2,4-dioxo-3,4-dihydropyrimidin-1(2H)-yl]acetyl}amino)methyl]benzyl}-carbamate.
(g) tert-Butyl {5-[({[3-{[(5-chloro-1,3-dimethyl-1H-pyrazol-4-yl)methyl]amino}-6-methyl-2,4-dioxo-3,4-dihydropyrimidin-1(2H)-yl]acetyl}amino)methyl]-4,6-dimethylpyridin-2-yl}carbamate.
(h) tert-Butyl {4-chloro-2-[({[3-{[(5-chloro-1,3-dimethyl-1H-pyrazol-4-yl)-methyl]amino}-6-methyl-2,4-dioxo-3,4-dihydropyrimidin-1(2H)-yl]acetyl}-amino)methyl]benzyl}carbamate.

### Example 5

Using procedures analogous to that set out in Example 2, and employing Boc-protected compounds from Example 4 above in place of tert-butyl {4-chloro-2-[({[3-[(2,2-difluoro-2-pyridin-2-ylethyl)amino]-6-methyl-2,4-dioxo-3,4-dihydro-pyrimidin-1(2H)-yl]acetyl}amino)methyl]benzyl}carbamate, the following compounds were prepared.
(a) N-[(6-Amino-2,4-dimethylpyridin-3-yl)methyl]-2-[3-[(2,2-difluoro-2-pyridin-2-ylethyl)amino]-6-methyl-2,4-dioxo-3,4-dihydropyrimidin-1(2H)-yl]acetamide hydrochloride salt.

¹H NMR (400 MHz, D₂O): d 8.69 (d, 1H), 8.35 (t, 1H), 7.97 (d, 1H), 7.86 (t, 1H), 6.48 (s, 1H), 5.65 (s, 1H), 4.46 (s, 2H), 4.21 (s, 2H), 3.68 (t, 2H), 2.34 (s, 3H), 2.21 (s, 3H), 2.07 (s, 3H)

HRMS (ESI) calculated for C₂₂H₂₆N₇O₃F₂ 474.2065 (M+H)⁺. found 474.2071.
(b) N-[(6-Amino-2,4-dimethylpyridin-3-yl)methyl]-2-[3-[(2-chloro-5-fluorobenzyl)amino]-6-methyl-2,4-dioxo-3,4-dihydropyrimidin-1(2H)-yl]acetamide hydrochloride salt.

¹H NMR (400 MHz, D₂O): d 8.42 (t, 1H), 7.23 (dd, 1H), 6.90 (dt, 1H), 6.84 (dd, 1H), 6.55 (s, 1H), 5.57 (s, 1H), 4.40 (s, 2H), 4.19 (s, 2H), 4.01 (s, 2H), 2.37 (s, 3H), 2.25 (s, 3H), 2.06 (s, 3H)

HRMS (ESI) calculated for C₂₂H₂₅N₆O₃FCl₂ 475.1661 (+H)⁺. found 475.1681.
(c) N-[2-(Aminomethyl)benzyl]-2-[3-[(2-chloro-5-fluorobenzyl)amino]-6-methyl-2,4-dioxo-3,4-dihydropyrimidin-1(2H)-yl]acetamide hydrochloride salt.

¹H NMR (400 MHz, CD₃OD): d 7.47-7.30 (m, 5H), 7.26 (dd, 1H), 7.00 (dt, 1H), 5.63 (s, 1H), 4.59 (s, 2H), 4.47 (s, 2H), 4.24 (s, 2H), 4.18 (s, 2H), 2.16 (s, 3H)

HRMS (ESI) calculated for C₂₂H₂₄N₅O₃Cl₂F 460.1552 (M+H)⁺. found 460.1537.
(d) N-[2-(Aminomethyl)-5-chlorobenzyl]-2-[3-[(2-chloro-5-fluorobenzyl)amino]-6-methyl-2,4-dioxo-3,4-dihydropyrimidin-1(2H)-yl]acetamide hydrochloride salt.

¹H NMR (400 MHz, CD₃OD): d 7.47 (d, 1H), 7.42-7.30 (m, 3H), 7.25 (dd, 1H), 7.00 (dd, 1H), 5.63 (s, 1H), 4.60 (s, 2H), 4.45 (s, 2H), 4.23 (s, 2H), 4.17 (s, 2H), 2.17 (s, 3H)

HRMS (ESI) calculated for C₂₂H₂₃N₅O₃Cl₂F 494.1162 (M+H)⁺. found 494.1151.
(e) N-[(6-Amino-2,4-dimethylpyridin-3-yl)methyl]-2-[3-{[(3,5-dimethylisoxazol-4-yl)methyl]amino}-6-methyl-2,4-dioxo-3,4-dihydropyrimidin-1(2H)-yl]-acetamide acetate salt.

¹H NMR (500 MHz, DMSO-d₆): d 8.20 (t, 1H), 6.13 (s, 1H), 5.97 (t, 1H), 5.70 (broad s, 2H), 5.64 (s, 1H), 4.44 (s, 2H), 4.18 (d, 2H), 3.78 (d, 2H), 2.28 (s, 3H), 2.23 (s, 3H), 2.22 (s, 3H), 2.14 (s, 3H), 2.13 (s, 3H).

HRMS (ESI) calculated for C₂₁H₂₇N₇O₄ 442.2203 (M+H)⁺. found 442.2192.
(f) N-[2-(Aminomethyl)-5-chlorobenzyl]-2-[3-{[(3,5-dimethylisoxazol-4-yl)-methyl]amino}-6-methyl-2,4-dioxo-3,4-dihydropyrimidin-1(2H)-yl]acetamide acetate salt.

¹H NMR (500 MHz, CD₃OD): d 7.48-7.37 (m, 1H), 5.70 (s, 1H), 4.63 (s, 2H), 4.48 (s, 2H), 4.20 (s, 2H), 3.91 (s, 2H), 2.30 (s, 6H), 2.22 (s, 3H).

HRMS (ESI) calculated for C₂₁H₂₅ClN₆O₄ 461.1704 (M+H)⁺. found 461.1707.
(g) N-[(6-Amino-2,4-dimethylpyridin-3-yl)methyl]-2-[3-{[(5-chloro-1,3-dimethyl-1H-pyrazol-4-yl)methyl]amino}-6-methyl-2,4-dioxo-3,4-dihydropyrimidin-1(2H)-yl]acetamide.
(h) N-[2-(Aminomethyl)-5-chlorobenzyl]-2-[3-{[(5-chloro-1,3-dimethyl-1H-pyrazol-4-yl)methyl]amino}-6-methyl-2,4-dioxo-3,4-dihydropyrimidin-1(2H)-yl]acetamide.

### Example 6

Compounds of the Examples were tested in Test B above and were found to exhibit IC₅₀TT values of less than 50 μM. Indeed, the compounds of Examples 2 and 5(a) were found to exhibit IC₅₀ values of 32.7 nM and 169 nM, respectively.

### ABBREVIATIONS

aq.=aqueous
AUC=area under the curve
Boc=tert-butyloxycarbonyl
BSA=bovine serum albumin
d=(in relation to NMR) doublet
DCC=dicyclohexyl carbodiimide
DCE=1,2-dichloroethane
DCM=dichloromethane
DEAD=diethylazodicarboxylate
DIPEA=diisopropylethylamine
DMAP=4-(N,N-dimethyl amino)pyridine
DMF=dimethylformamide
DMSO=dimethylsulfoxide
DVT=deep vein thrombosis
EDC=1-(3-dimethylaminopropyl)-3-ethylcarbodiimide hydrochloride
ESI electron spray ionisation
Et=ethyl
ether=diethyl ether
Et₃N=triethylamine
EtOAc=ethyl acetate
EtOH=ethanol
Et₂O=diethyl ether
h=hour(s)
HATU=O-(azabenzotriazol 1-yl)-N,N,N′,N′-tetramethyluronium hexafluorophosphate
HBTU=[N,N,N′,N′-tetramethyl-O-(benzotriazol-1-yl)uronium hexafluorophosphate]
HCl hydrochloric acid, hydrogen chloride gas or hydrochloride salt (depending on context)
HOAt=1-hydroxy-7-azabenzotriazole
HOBt=1-hydroxybenzotriazole
HPLC=high performance liquid chromatography
HRMS=high resolution mass spectrometry
LC=liquid chromatography mCPBA meta-chloroperbenzoic acid
Me=methyl
MeCN=acetonitrile
MeOH=methanol
min=minute(s)
MS=mass spectroscopy
NADH=nicotinamide adenine dinucleotide, reduced form
NADPH=nicotinamide adenine dinucleotide phosphate, reduced form

### NBS=N-Bromosuccinimide

### NIH=National Institute of Health (US)

NIHU=National Institute of Health units
OAc=acetate
PCC=pyridinium chlorochromate
Ph=phenyl
Pr=propyl
PyBOP=(benzotriazol 1-yloxy)tripyrrolidinophosphonium hexafluorophosphate
rt/RT=room temperature
SOPs=standard operating procedures
TBA=tetrabutylammonium
TBME=tert-butyl methyl ether
TBTU=[N,N,N′,N′-tetramethyl-O-(benzotriazol-1-yl)uronium tetrafluoroborate]
TEA=triethylamine
TFA=trifluoroacetic acid
THF=tetrahydrofuran

Prefixes n, s, i and t have their usual meanings: normal, secondary, iso and tertiary. The prefix c means cyclo.

1. A compound of formula I 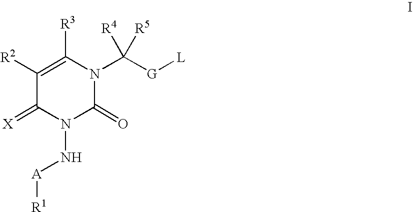
wherein
X represents O or S;
A represents C(O), S(O)₂, C(O)O (in which latter group the O moiety is attached to R¹), C(O)NH, S(O)₂NH (in which latter two groups the NH moiety is attached to R¹), a direct bond or C₁₋₆ alkylene (which latter group is optionally substituted, at the C-atom to which the NH moiety is attached, by C(O)OR^{A} or C(O)N(H)R^{A});
R^{A} represents H or C₁₋₄ alkyl;
R¹ represents
(a) C₁₋₁₀ alkyl, C₂₋₁₀ alkenyl, C₂₋₁₀ alkynyl (which latter three groups are optionally substituted by one or more substituents selected from halo, CN, C₃₋₁₀ cycloalkyl (optionally substituted by one or more substituents selected from halo, OH, ═O, C₁₋₆ alkyl, C₁₋₆ alkoxy and aryl), OR^{6a}, S(O)ₙR^{6b}, S(O)₂N(R^{6c})(R^{6d}), N(R^{6e})S(O)₂R⁶, N(R^{6g})(R^{6h}), B¹—C(O)—B²—R⁶ⁱ, aryl and Het¹),
(b) C₃₋₁₀ cycloalkyl or C₄₋₁₀ cycloalkenyl, which latter two groups are optionally substituted by one or more substituents selected from halo, ═O, CN, C₁₋₁₀ alkyl, C₃₋₁₀ cycloalkyl (optionally substituted by one or more substituents selected from halo, OH, ═O, C₁₋₆ alkyl, C₁₋₆ alkoxy and aryl), OR^{6a}, S(O)R^{6b}, S(O)₂N(R^{6c})(R^{6d}), N(R^{6e})S(O)₂R^{6f}, N(R^{6g})(R^{6h}), B³—C(O)—B⁴—R⁶ⁱ, aryl and Het²,
(c) aryl, or
(d) Het³;
R^{6a} to R⁶ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, aryl and Het⁴),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, aryl and Het⁵),
(d) aryl or
(e) Het⁶,
provided that R^{6b} does not represent H when n is 1 or 2;
R² represents H or halo;
R³ represents
(a) H,
(b) halo,
(c) CN,
(d) C₁₋₆ alkyl, C₂₋₆ alkenyl, C₂₋₆ alkynyl, C₁₋₆ alkoxy (which latter four groups are optionally substituted by one or more substituents selected from halo, OH, CN, C₁₋₄ alkoxy, C(O)OH, C(O)O—C₁₋₄ alkyl and OC(O)—C₁₋₄ alkyl) or
(e) together with R⁴, R³ represents C₂₋₃ n-alkylene, T¹-(C₁₋₂ n-alkylene) or (C₁₋₂ n-alkylene)-T¹, which latter three groups are optionally substituted by halo, or
(f) together with R⁴ and R⁵, R³ represents T²-[C(H)═], wherein T² is bonded to the C-atom to which the group R³ is attached;
R⁴ and R⁵ independently represent H, F or methyl (which latter group is optionally substituted by one or more F atoms), or
(a) together with R³, R⁴ represents C₂₋₃ n-alkylene, T¹-(C₁₋₂ n-alkylene) or (C₁₋₂ n-alkylene)-T¹, which latter three groups are optionally substituted by halo, or
(b) together with R³, R⁴ and R⁵ represent T²-[C(H)═], wherein T² is bonded to the C-atom to which the group R³ is attached;
T¹ and T² independently represent O, S, or NR⁷;
R⁷ represents H or C₁₋₄ alkyl;
G represents
(a) —C(R^{7a})(R^{7b})N(R^{8a})—[CH(C(O)R⁹)]₀₋₁—C₀₋₃ alkylene-(Q¹)ₐ-,
(b) —C(R^{7a})(R^{7b})(O)N(R^{8b})—C₂₋₃ alkenylene-(Q¹)ₐ-, 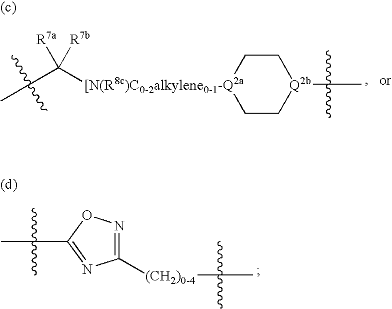
R^{7a} and R^{7b} independently represent H or methyl, or R^{7a} and R^{7b} together represent ═O;
R⁹ represents H or a 5- to 10-membered aromatic heterocyclic group comprising one or two rings and containing, as heteroatom(s), one sulfur or oxygen atom and/or one or more nitrogen atoms, which heterocyclic group is optionally substituted by one or more substituents selected from halo and C₁₋₆ alkyl;
Q¹ represents O, NR^{10a}, [N(H)]₀₋₁C(O)—C₀₋₂ alkylene, C(O)NHNHC(O), or —N═C(R^{10b})—;
a represents 0 or 1;
Q^{2a} represents 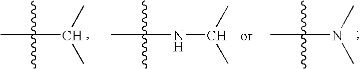
Q^{2b} represents 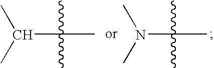
L represents
(a) C₀₋₆ alkylene-R^{a},
(b) C₀₋₂ alkylene-CH═CH—C₀₋₂ alkylene-R^{a},
(c) C₀₋₂ alkylene-C≡C—C₀₋₂ alkylene-R^{a}, 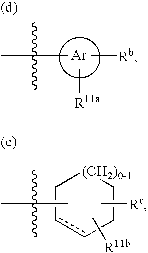
wherein the dashed line represents an optional double bond, or 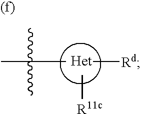
Ar represents phenyl or naphthyl;
Het represents a 5- to 10-membered heterocyclic group comprising one or two rings and containing, as heteroatom(s), one sulfur or oxygen atom and/or one or more nitrogen atoms;
R^{11a} represents H or one or more substituents selected from halo, OH, CN, C₁₋₆ alkyl, C₁₋₆ alkoxy (which latter two groups are optionally substituted by one or more substituents selected from halo, OH, C₁₋₄ alkoxy, C(O)OR^{12a} and C(O)N(R^{12b})R^{2c} and S(O)₀₋₂R^{12d};
R^{11b} and R^{11c} independently represent H or one or more substituents selected from halo, OH, CN, C₁₋₆ alkyl, C₁₋₆ alkoxy (which latter two groups are optionally substituted by one or more substituents selected from halo, OH, C₁₋₄ alkoxy, C(O)OR^{12a} and C(O)N(R^{12b})R^{12c}, S(O)₀₋₂R^{12d}, ═O, ═NH, ═NOH and ═N—CN;
R^{12a} to R^{12c} independently represent H, C₁₋₆ alkyl or C₃₋₇ cycloalkyl (which latter two groups are optionally substituted by one OH or N(R^{12e})R^{12f} group or by one or more halo atoms);
R^{12d} represents, independently at each occurrence, C₁₋₆ alkyl optionally substituted by one OH or N(R^{12e})R^{12f} group or by one or more halo atoms;
R^{12e} and R^{12f} represent, independently at each occurrence, H or C₁₋₄ alkyl optionally substituted by one or more halo atoms;
R^{a} to R^{d} independently represent 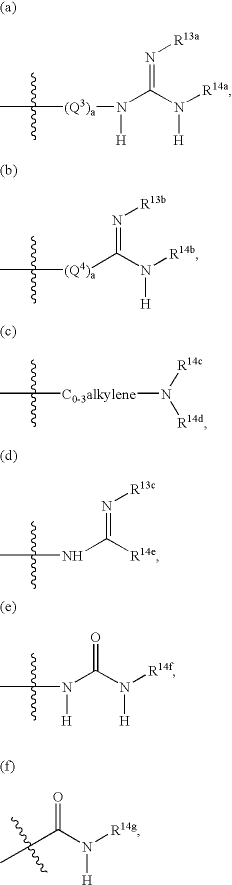
(g) Het^{x}
or R^{b} to R^{d} may also represent H;
Q³ represents O, N(R^{10c}), S(O)₂, S(O)₂NH, C(O) or —CH═N—;
Q⁴ represents O, S or CH₂;
Het^{x} represents a 5- or 6-membered heterocyclic group containing one to four heteroatoms selected from oxygen, nitrogen and/or sulfur, which heterocyclic group may be substituted by one or more substituents selected from halo, ═O, C₁₋₆ alkyl and C₁₋₆ alkoxy (which latter two groups are optionally substituted by one or more halo atoms);
R^{13a} to R^{13c} independently represent
(a) H,
(b) CN,
(c) NH₂,
(d) OR¹⁵ or
(e) C(O)OR¹⁶;
R¹⁵ represents
(a) H,
(b) C₁₋₁₀ alkyl, C₃₋₁₀ alkenyl, or C₃₋₁₀ alkynyl,
(c) C₃₋₁₀ cycloalkyl, C₄₋₁₀ cycloalkenyl, which latter two groups are optionally substituted by one or more substituents selected from halo and C₁₋₆ alkyl, or
(d) C₁₋₃ alkyl, which latter group is optionally interrupted by oxygen and is substituted by aryl or —O-aryl;
R¹⁶ represents
(a) C₁₋₁₀ alkyl, C₃₋₁₀ alkenyl, C₃₋₁₀ alkynyl, which latter three groups are optionally interrupted by one or more oxygen atoms, or
(b) C₃₋₁₀ cycloalkyl, C₄₋₁₀ cycloalkenyl, which latter two groups are optionally substituted by one or more substituents selected from halo and C₁₋₆ alkyl, or
(c) C₁₋₃ alkyl, which latter group is optionally interrupted by oxygen and is substituted by aryl or —O-aryl;
R^{8a} to R^{8c}, R^{10a} to R^{10c} and R^{14a} to R^{14g} independently represent
(a) H or
(b) C₁₋₄ alkyl (which latter group is optionally substituted by one or more substituents selected from halo and OH),
or R^{14a} and R^{14b} independently represent C(O)O—C₁₋₁₆ alkyl (the alkyl part of which latter group is optionally substituted by aryl and/or one or more halo atoms),
or R^{14c} represents
(a) C₁₋₄ alkyl substituted by C₃₋₇ cycloalkyl or aryl,
(b) C₃₋₇ cycloalkyl,
(c) C(O)O—C₁₋₆ alkyl (the alkyl part of which latter group is optionally substituted by aryl and/or one or more halo atoms),
(d) C(O)C₁₋₆ alkyl,
(e) C(O)N(H)—C₁₋₆ alkyl (the alkyl part of which latter group is optionally substituted by aryl and/or one or more halo atoms) or
(f) S(O)₂—C₁₋₆ alkyl (the alkyl part of which latter group is optionally substituted by aryl and/or one or more halo atoms),
or R^{14c} and R^{14d} together represent C₃₋₆ n-alkylene optionally interrupted by O, S, N(H) or N(C₁₋₄ alkyl) and/or substituted by one or more C₁₋₄ alkyl groups;
each aryl independently represents a C₆₋₁₀ carbocyclic aromatic group, which group may comprise either one or two rings and may be substituted by one or more substituents selected from
(a) halo,
(b) CN,
(c) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, C(O)OH, C(O)O—C₁₋₆ alkyl, phenyl (which latter group is optionally substituted by halo) and Het⁷),
(d) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het⁸),
(e) OR^{17a},
(f) S(O)ₚR^{17b}
(g) S(O)₂N(R^{17c})(R^{17d}),
(h) N(R^{17e})S(O)₂R^{17f},
(i) N(R^{17g})(R^{17h}),
(j) B⁵—C(O)—B⁶—R¹⁷ⁱ
(k) phenyl (which latter group is optionally substituted by halo),
(l) Het⁹ and
(m) Si(R^{18a})(R^{18b})(R^{18c});
R^{17a} to R¹⁷ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, phenyl (which latter group is optionally substituted by halo) and Het¹⁰),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het¹¹),
(d) phenyl (which latter group is optionally substituted by halo) or
(e) Het¹²,
provided that R^{17b} does not represent H when p is 1 or 2;
Het¹ to Het¹² independently represent 4- to 14-membered heterocyclic groups containing one or more heteroatoms selected from oxygen, nitrogen and/or sulfur, which heterocyclic groups may comprise one, two or three rings and may be substituted by one or more substituents selected from
(a) halo,
(b) CN,
(c) C₁₋₁₀ alkyl, C₂₋₁₀ alkenyl, C₂₋₁₀ alkynyl (which latter four groups are optionally substituted by one or more substituents selected from halo, OH, C₁₋₆ alkoxy, C(O)OH, C(O)O—C₁₋₆ alkyl, phenyl (which latter group is optionally substituted by halo) and Het^{a}),
(d) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het^{b}),
(e) ═O,
(f) OR^{19a},
(g) S(O)_{q}R^{19b},
(h) S(O)₂N(R^{19c})(R^{19d}),
(i) N(R^{19e})S(O)₂R^{19f},
(j) N(R^{19g})(R^{19h}),
(k) B⁷—C(O)—B⁸—R¹⁹ⁱ,
(l) phenyl (which latter group is optionally substituted by halo),
(m) Het^{c} and
(n) Si(R^{20a})(R^{20b})(R^{20c});
R^{19a} to R¹⁹ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, phenyl (which latter group is optionally substituted by halo) and Het^{d}),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het^{e}),
(d) phenyl (which latter group is optionally substituted by halo) or
(e) Het^{e},
provided that R^{19b} does not represent H when q is 1 or 2;
Het^{a} to Het^{f} independently represent 5- or 6-membered heterocyclic groups containing one to four heteroatoms selected from oxygen, nitrogen and/or sulfur, which heterocyclic groups may be substituted by one or more substituents selected from halo, ═O and C₁₋₆ alkyl;
B¹ to B⁸ independently represent a direct bond, O, S, NH or N—C₁₋₄ alkyl;
n, p and q independently represent 0, 1 or 2;
R^{18a}, R^{18b}, R^{18c}, R^{20a}, R^{20b} and R^{20c} independently represent C₁₋₆ alkyl or phenyl (which latter group is optionally substituted by halo or C₁₋₄ alkyl);
unless otherwise specified
(i) alkyl, alkenyl, alkynyl, cycloalkyl, cycloalkenyl, alkylene and alkenylene groups, as well as the alkyl part of alkoxy groups, may be substituted by one or more halo atoms, and
(ii) cycloalkyl and cycloalkenyl groups may comprise one or two rings and may additionally be ring-fused to one or two phenyl groups;
or a pharmaceutically-acceptable derivative thereof.

2. A compound as claimed in claim 1, which is a compound of formula Ia, 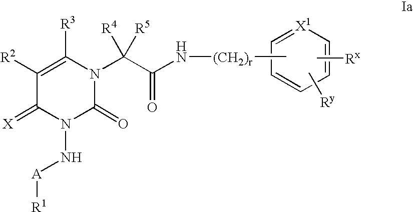
wherein X¹ represents CH or N;
when X¹ represents CH
(a) R^{x} takes the same definitions as R^{b} as defined in claim 1, and
(b) R^{y} takes the same definitions as R^{11a} as defined in claim 1;
when X¹ represents N
(a) R^{x} takes the same definitions as R^{d} as defined in claim 1, and
(b) R^{y} takes the same definitions as R^{11e} as defined in claim 1;
r represents 1 to 3; and
R¹ to R⁵, R^{11a}, R^{11c}, R^{b}, R^{d}, A and X are as defined in claim 1,

3. A compound as claimed in claim 2, wherein:
X represents O;
A represents (CH₂)₂, CH₂ or CF₂CH₂ (in which latter group the CF₂ unit is attached to R¹);
R¹ represents
(a) phenyl optionally substituted by one or two substituents selected from halo and methyl,
(b) isoxazol-4-yl optionally substituted by one or two methyl substituents,
(c) pyrazol-4-yl optionally substituted by one to three substituents selected from Cl and methyl, or
(d) pyridinyl optionally substituted by OH or halo;
R² represents H or F;
R³ represents methyl;
R⁴ and R⁵ both represent H;
r represents 1 or 2;
the group 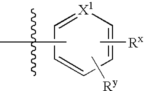
represents 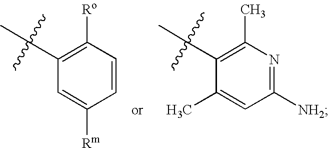
wherein R^{o} represents tetrazol-1-yl, OCH₂C(O)N(H)R^{12b} or CH₂NH₂;
R^{m} represents H or Cl; and
R^{12b} represents C₁₋₃ alkyl.

4. A compound as claimed in claim 1 wherein X represents S and R³ represents CN or C₁₋₄ alkyl substituted by one or more fluoro atoms.

5. A pharmaceutical formulation comprising a compound according to claim 1, or a pharmaceutically acceptable derivative thereof, in admixture with a pharmaceutically acceptable adjuvant, diluent or carrier.

6-7. (canceled)

8. A method of treatment of a hypercoagulability and/or thrombo-embolic disease or condition, which method comprises administration of a therapeutically effective amount of a compound according to claim 1, or a pharmaceutically acceptable derivative thereof, to a person suffering from, or susceptible to, such a disease or condition.

9. A process for the preparation of a compound of formula I as defined in claim 1, which comprises:
(a) for compounds of formula I in which R^{7a} and R^{7b} together represent ═O, coupling of a compound of formula II, 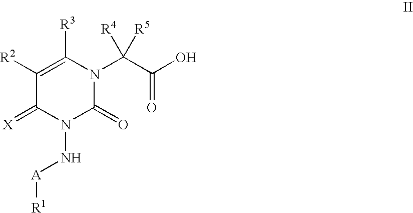
wherein R¹ to R⁵, A and X are as defined in claim 1, with a compound of formula III,
H-G^{a}-L III
wherein L is as defined in claim 1 and G^{a} represents
(i) —N(R^{8a})—[CH(C(O)R⁹)]₀₋₁—C₀₋₃ alkylene-(Q¹)ₐ-,
(ii) —N(R^{8b})—C₂₋₃ alkenylene-(Q¹)ₐ-,
(iii) —N(R^{8b})—C₂₋₃ alkynylene-(Q¹)ₐ-, 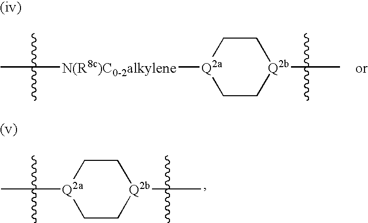
wherein Q^{2a} represents N or NHCH and R^{8a}, R^{8b}, R^{8c}, R⁹, Q¹, Q^{2b} and a are as defined in claim 1;
(b) for compounds of formula I in which R^{7a} and R^{7b} independently represent H or methyl, reaction of a compound of formula IV, 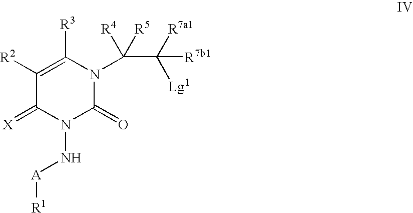
wherein R^{7a1} and R^{7b1} independently represent H or methyl, Lg¹ represents a leaving group and R¹ to R⁵, A and X are as defined in claim 1, with a compound of formula III, as defined above;
(c) for compounds of formula I in which R^{7a} represents H and R^{7b} represents H or methyl, reaction of a compound of formula V, 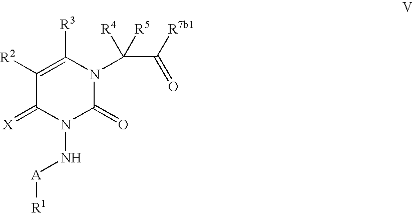
wherein R¹ to R⁵, A and X are as defined in claim 1 and R^{7b1} is as defined above, with a compound of formula III, as defined above, followed by reduction in the presence of a reducing agent;
(d) for compounds of formula I in which G represents 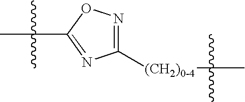
and L represents L^{a}, which latter group represents L as defined in claim 1, except that it does not represent C₀ alkylene-R^{a}, cyclisation of a compound of formula VI, 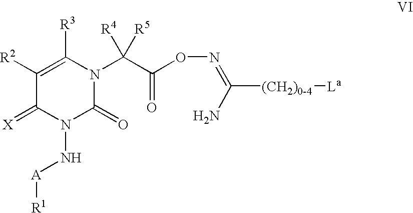
wherein R¹ to R⁵, A and X are as defined in claim 1 and L^{a} is as defined above;
(e) for compounds of formula I in which R^{a}, R^{b}, R^{c} or R^{d} represents —C(═NH)NH₂, —C(═NNH₂)NH₂ or —C(═NOH)NH₂, reaction of a compound of formula VII, 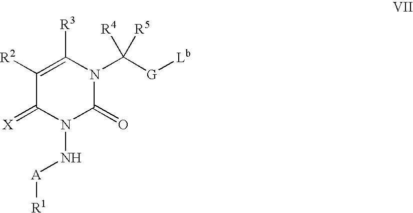
wherein L^{b} represents L as defined in claim 1, except that R^{a}, R^{b}, R^{c} or R^{d} (as appropriate) is replaced by a cyano or —C(═NH)O—C₁₋₄ alkyl group, and R¹ to R⁵, A, G and X are as defined in claim 1, with a suitable source of ammonia, hydrazine or hydroxylamine;
(f) for compounds of formula I in which R^{13a}, R^{13b} or R^{13c} represents H, deprotection of a corresponding compound of formula I in which R^{13a}, R^{13b} or R^{13c} (as appropriate) represents C(O)O—CH₂aryl;
(g) for compounds of formula I in which R^{14c} represents H, deprotection of a corresponding compound of formula I in which R^{14c} represents C(O)O—C₁₋₆ alkyl;
(h) reaction of a compound of formula VIII, 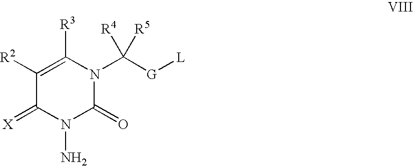
wherein R² to R⁵, G, L and X are as defined in claim 1, with a compound of formula IX,
R¹-A-Lg² IX
wherein Lg² represents a leaving group and R¹ and A are as defined in claim 1;
(i) for compounds of formula I in which A represents C(O)NH, reaction of a compound of formula VIII, as defined above, with a compound of formula X,
R¹—N═C═O X
wherein R¹ is as defined in claim 1;
(j) for compounds of formula I in which A represents C₁₋₆ alkylene, reaction of a compound of formula VIII, as defined above, with a compound of formula XI,
R¹—C₀₋₅ alkylene-CHO XI
wherein R¹ is as defined in claim 1, followed by reduction in the presence of a reducing agent;
(k) for compounds of formula I in which R^{a}, R^{b}, R^{c} or R^{d} represents —C(═NCN)NH₂, reaction of a corresponding compound of formula I in which R^{a}, R^{b}, R^{c} or R^{d}, respectively, represents —C(═NH)NH₂ with cyanogen bromide;
(l) reaction of a compound of formula XII, 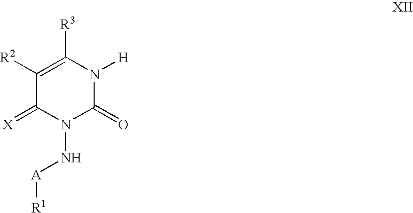
wherein R¹, R², R³, A and X are as defined in claim 1, with a compound of formula XIII, 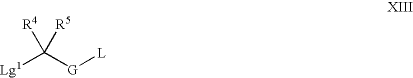
wherein R⁴, R⁵, G and L are as defined in claim 1 and Lg¹ is as defined above, in the presence of a base;
(m) reaction of a compound of formula XII, as defined above, with a compound of formula XIV, 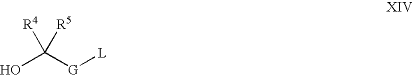
wherein R⁴, R⁵, G and L are as defined in claim 1, under Mitsunobu conditions; or
(f) deprotection of a protected derivative of a compound as claimed in claim 1.

10. A compound of formula II, 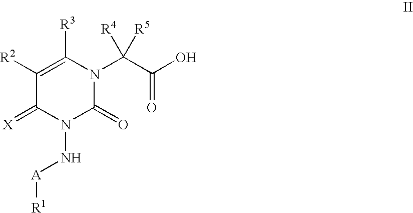
wherein:
X represents O or S;
A represents C(O), S(O)₂, C(O)O (in which latter group the O moiety is attached to R¹), C(O)NH, S(O)₂NH (in which latter two groups the NH moiety is attached to R¹), a direct bond or C₁₋₆ alkylene (which latter group is optionally substituted, at the C-atom to which the NH moiety is attached by C(O)OR^{A} or C(O)N(H)R^{A});
R^{A} represents H or C₁₋₄ alkyl
R¹ represents
(a) C₁₋₁₀ alkyl C₂₋₁₀ alkenyl, C₂₋₁₀ alkynyl (which latter three groups are optionally substituted by one or more substituents selected from halo, CN, C₃₋₁₀ cycloalkyl (optionally substituted by one or more substituents selected from halo, OH, ═O, C₁₋₆ alkyl, C₁₋₆ alkoxy and aryl), OR^{6a}, S(O)ₙR^{6b}, S(O)₂N(R^{6c})(R^{6d}), N(R^{6e})S(O)₂R^{6f}, N(R^{6g})(R^{6h}) B¹—C(O)—B²—R⁶ⁱ, aryl and Het¹)
(b)C₃₋₁₀ cycloalkyl or C₄₋₁₀ cycloalkenyl (which latter two groups are optionally substituted by one or more substituents selected from halo, ═O, CN, C₁₋₁₀ alkyl, C₃₋₁₀ cycloalkyl (optionally substituted by one or more substituents selected from halo, OH, ═O, C₁₋₆ alkyl, C₁₋₆ alkoxy and aryl), OR^{6a}, S(O)ₙR^{6b}, S(O)N(R^{6c})(R^{6d}) N(R^{6e})S(O)₂R^{6f}, N(R^{6g})(R^{6h}) B³—C(O)—B⁴—R⁶ⁱ, aryl and Het²)
(c) aryl, or
(d) Het³;
R^{6a} to R⁶ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, aryl and Het⁴),
(c) C₃₋₁₀ cycloalkyl, C₄₋₁₀ cycloalkenyl (which latter two groups are optionally substituted by one or more substituents selected from halo, OH, ═O, C₁₋₆ alky, C₁₋₆ alkoxy, ary and Het⁵),
(d) aryl or
(e) Het⁶,
provided that R^{6b} does not represent H when n is 1 or 2;
R² represents H or halo;
R³ represents
(a) H,
(b) halo,
(c) CN,
(d) C₁₋₆ alkyl, C₂₋₆ alkenyl, C₂₋₆ alkynyl, C₁₋₆ alkoxy (which latter four groups are optionally substituted by one or more substituents selected from halo, OH, CN, C₁₋₄ alkoxy, C(O)OH, C(O)O—C₁₋₄ alkyl and OC(O)—C₁₋₄ alky) or
(e) together with R⁴, R³ represents C₂₋₃ n-alkylene, T¹-(C₁₋₂ n-alkylene) or (C₁₋₂ n-alkylene)-T¹, which latter three groups are optionally substituted by halo, or
(f) together with R⁴ and R⁵, R³ represents T²-[C(H)═], wherein T² is bonded to the C-atom to which the group R³ is attached;
R⁴ and R⁵ independently represent H, F or methyl (which latter group is optionally substituted by one or more F atoms), or
(a) together with R³, R⁴ represents C₂₋₃ n-alkylene, T¹-(C₁₋₂ n-alkylene) or (C₁₋₂ n-alkylene)-T¹, which latter three groups are optionally substituted by halo, or
(b) together with R³, R⁴ and R⁵ represent T²-[C(H)═], wherein T² is bonded to the C-atom to which the group R³ is attached;
T¹ and T² independently represent O, S, or NR⁷;
R⁷ represents H or C₁₋₄ alkyl;
n, p, and q, independently, represent 0, 1 or 2;
each aryl independently represents a C₆₋₁₀ carbocyclic aromatic group, which group may comprise either one or two rings and may be substituted by one or more substituents selected from
(a) halo,
(b) CN,
(c) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, C(O)OH, C(O)O—C₁₋₆ alkyl, phenyl (which latter group is optionally substituted by halo) and Het⁷),
(d) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het⁷),
(e) OR^{17a},
(f) S(O)ₚR^{17b},
(g) S(O)₂N(R^{17c})(R^{17d}),
(h) N(R^{17e})S(O)₂R^{17f},
(i) N(R^{17g})(R^{17h}),
(j) B⁵—C(O)—B⁶—R¹⁷ⁱ,
(k) phenyl (which latter group is optionally substituted by halo),
(l) Het⁹ and
(m) Si(R^{18a})(R^{18b})(R^{18c});
R^{17a} to R¹⁷ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, phenyl (which latter group is optionally substituted by halo) and Het¹⁰),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het¹¹),
(d) phenyl (which latter group is optionally substituted by halo) or
(e) Het¹²,
provided that R^{17b} does not represent H when p is 1 or 2;
B¹ to B⁸ independently represent a direct bond, O, S, NH or N—C₁₋₄ alkyl;
Het¹ to Het¹² independently represent 4- to 14-membered heterocyclic groups containing one or more heteroatoms selected from oxygen, nitrogen and/or sulfur, which heterocyclic groups may comprise one, two or three rings and may be substituted by one or more substituents selected from
(a) halo,
(b) CN,
(c) C₁₋₁₀ alkyl, C₂₋₁₀ alkenyl, C₂₋₁₀ alkynyl (which latter four groups are optionally substituted by one or more substituents selected from halo, OH, C₁₋₆ alkoxy, C(O)OH, C(O)O—C₁₋₆ alkyl, phenyl (which latter group is optionally substituted by halo) and Het^{a}),
(d) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het^{b}),
(e) ═O,
(f) OR^{19a},
(g) S(O)_{q}R^{19b},
(h) S(O)₂N(R^{19c})(R^{19d}),
(i) N(R^{19e})S(O)₂R^{19f},
(j) N(R^{19g})(R^{19h}),
(k) B⁷—C(O)—B⁸—R¹⁹ⁱ,
(l) phenyl (which latter group is optionally substituted by halo),
(m) Het^{c} and
(n) Si(R^{20a})(R^{20b})(R^{20c});
R^{19a} to R¹⁹ⁱ independently represent, at each occurrence
(a) H,
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, phenyl (which latter group is optionally substituted by halo) and Het^{d}),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het^{e}),
(d) phenyl (which latter group is optionally substituted by halo) or
(e) Het^{f},
provided that R^{19b} does not represent H when q is 1 or 2;
Het^{a} to Het^{f} independently represent 5- or 6-membered heterocyclic groups containing one to four heteroatoms selected from oxygen, nitrogen and/or sulfur, which heterocyclic groups may be substituted by one or more substituents selected from halo, ═O and C₁₋₆ alkyl;
R^{18a}, R^{18b}, R^{18c}, R^{20a}, R^{20b} and R^{20c} independently represent C₁₋₆ alkyl or phenyl (which latter group is optionally substituted by halo or C₁₋₄ alkyl
unless otherwise specified
(i) alkyl, alkenyl, alkynyl, cycloalkyl, cycloalkenyl, alkylene and alkenylene groups, as well as the alkyl part of alkoxy groups, may be substituted by one or more halo atoms, and
(ii) cycloalkyl and cycloalkenyl groups may comprise one or two rings and may additionally be ring-fused to one or two phenyl groups;
or a protected derivative thereof.

11. A compound of formula IV, 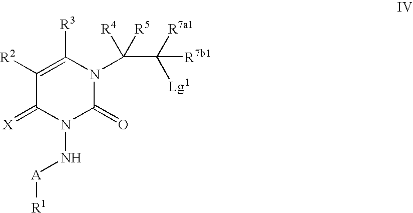
wherein
R^{7a1} and R^{7b1} independently represent H or methyl;
Lg¹ represents a leaving group;
X represents O or S;
A represents C(O), S(O)₂, C(O)O (in which latter group the O moiety is attached to R¹), C(O)NH, S(O)₂NH (in which latter two groups the NH moiety is attached to R¹), a direct bond or C₁₋₆ alkylene (which latter group is optionally substituted at the C-atom to which the NH moiety is attached, by C(O)OR^{A} or C(O)N(H)R^{A});
R^{A} represents H or C₁₋₄ alkyl;
R¹ represents
(a) C₁₋₁₀ alkyl, C₂₋₁₀ alkenyl, C₂₋₁₀ alkynyl (which latter three groups are optionally substituted by one or more substituents selected from halo, CN, C₃₋₁₀ cycloalkyl (optionally substituted by one or more substituents selected from halo, OH, ═O, C₁₋₆ alkyl, C₁₋₆ alkoxy and aryl), OR^{6a}, S(O)ₙR^{6b}, S(O)₂(R^{6c})(R^{6d}), N(R^{6e})S(O)₂R^{6f}, N(R^{6g})(R^{6h}), B¹—C(O)—B²—R⁶ⁱ, aryl and Het¹),
(b) C₃₋₁₀ cycloalkyl or C₄₋₁₀ cycloalkenyl, which latter two groups are optionally substituted by one or more substituents selected from halo, ═O, CN, C₁₋₁₀ alkyl, C₃₋₁₀ cycloalkyl (optionally substituted by one or more substituents selected from halo, OH, ═O, C₁₋₆ alkyl, C₁₋₆ alkoxy and aryl) OR^{6a}, S(O)ₙR^{6b}, S(O)₂N(R^{6c})(R^{6d}), N(R^{6e})S(O)₂R^{6f}, N(R^{6g})(R^{6h}), B³—C(O)—B⁴—R⁶ⁱ, aryl and Het²,
(c) aryl or
(d) Het³;
R^{6a} to R⁶ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, aryl and Het⁴),
(c) C₃₋₁₀ cycloalkyl, C₄₋₁₀ cycloalkenyl (which latter two groups are optionally substituted by one or more substituents selected from halo, OH, ═O, C₁₋₆ alky, C₁₋₆ alkoxy, ary and Het⁵),
(d) aryl or
(e) Het⁶,
provided that R^{6b} does not represent H when n is 1 or 2;
R² represents H or halo;
R³ represents
(a) H,
(b) halo,
(c) CN,
(d) C₁₋₆ alkyl, C₂₋₆ alkenyl, C₂₋₆ alkynyl C₁₋₆ alkoxy (which latter four groups are optionally substituted by one or more substituents selected from halo, OH, CN, C₁₋₄ alkoxy, C(O)OH, C(O)O—C₁₋₄ alkyl and OC(O)—C₁₋₄ alky) or
(e) together with R⁴R³ represents C₂₋₃ n-alkylene, T¹-(C₁₋₂ n-alkylene) or (C₁₋₂ n-alkylene)-T¹, which latter three groups are optionally substituted by halo, or
(f) together with R⁴ and R⁵, R³ represents T²-[C(H)═], wherein T² is bonded to the C-atom to which the group R³ is attached;
R⁴ and R⁵ independently represent H, F or methyl (which latter group is optionally substituted by one or more F atoms) or
(a) together with R³, R⁴ represents C₂₋₃ n-alkylene, T¹-(C₁₋₂ n-alkylene) or (C₁₋₂ n-alkylene)-T¹, which latter three groups are optionally substituted by halo, or
(b) together with R³, R⁴ and R⁵ represent T²-[C(H)═], wherein T² is bonded to the C-atom to which the group R³ is attached;
T¹ and T² independently represent O, S, or NR⁷;
R⁷ represents H or C₁₋₄ alkyl;
n, p, and g, independently, represent 0, 1 or 2;
each aryl independently represents a C₆₋₁₀ carbocyclic aromatic group, which group may comprise either one or two rings and may be substituted by one or more substituents selected from
(a) halo,
(b) CN,
(c) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, C(O)OH, C(O)O—C₁₋₆ alkyl, phenyl (which latter group is optionally substituted by halo) and Het⁷),
(d) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het⁸),
(e) OR^{17a},
(f) S(O)ₚR^{17b},
(g) S(O)₂N(R^{17c})(R^{17d}),
(h) N(R^{17e})S(O)₂R^{17f},
(i) N(R^{17g})(R^{17h}),
(j) B⁵—C(O)—B⁶—R¹⁷ⁱ,
(k) phenyl (which latter group is optionally substituted by halo),
(l) Het⁹ and
(m) Si(R^{18a})(R^{18b})(R^{18c});
R^{17a} to R¹⁷ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, phenyl (which latter group is optionally substituted by halo) and Het¹⁰),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het¹¹),
(d) phenyl (which latter group is optionally substituted by halo) or
(e) Het¹²,
provided that R^{17b} does not represent H when p is 1 or 2;
B¹ to B⁸ independently represent a direct bond, O, S, NH or N—C₁₋₄ alkyl;
Het¹ to Het¹² independently represent 4- to 14-membered heterocyclic groups containing one or more heteroatoms selected from oxygen, nitrogen and/or sulfur, which heterocyclic groups may comprise one, two or three rings and may be substituted by one or more substituents selected from
(a) halo,
(b) CN,
(c) C₁₋₁₀ alkyl, C₂₋₁₀ alkenyl, C₂₋₁₀ alkynyl (which latter four groups are optionally substituted by one or more substituents selected from halo, OH, C₁₋₆ alkoxy, C(O)OH, C(O)O—C₁₋₆ alkyl, phenyl (which latter group is optionally substituted by halo) and Het^{a}),
(d) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het^{b}),
(e) ═O
(f) OR^{19a},
(g) S(O)_{q}R^{19b},
(h) S(O)₂N(R^{19c})(R^{19d}),
(i) N(R^{19e})S(O)₂R^{19f},
(j) N(R^{19g})(R^{19h}),
(k) B⁷—C(O)—B⁸—R¹⁹ⁱ,
(l) phenyl (which latter group is optionally substituted by halo),
(m) Het^{c} and
(n) Si(R^{20a})(R^{20b})(R^{20c});
R^{19a} to R¹⁹ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, phenyl (which latter group is optionally substituted by halo) and Het^{d}),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het^{e}),
(d) phenyl (which latter group is optionally substituted by halo) or
(e) Het^{f},
provided that R^{19b} does not represent H when q is 1 or 2;
Het^{a} to Het^{f} independently represent 5- or 6-membered heterocyclic groups containing one to four heteroatoms selected from oxygen, nitrogen and/or sulfur, which heterocyclic groups may be substituted by one or more substituents selected from halo, ═O and C₁₋₆ alkyl;
R^{18a}, R^{18b}, R^{18c}, R^{20a}, R^{20b} and R^{20c} independently represent C₁₋₆ alkyl or phenyl (which latter group is optionally substituted by halo or C₁₋₄ alkyl);
unless otherwise specified
(i) alkyl, alkenyl, alkynyl, cycloalkyl, cycloalkenyl, alkylene and alkenylene groups, as well as the alkyl part of alkoxy groups may be substituted by one or more halo atoms, and
(ii) cycloalkyl and cycloalkenyl groups may comprise one or two rings and may additionally be ring-fused to one or two phenyl groups;
or a protected derivative thereof.

12. A compound of formula V, 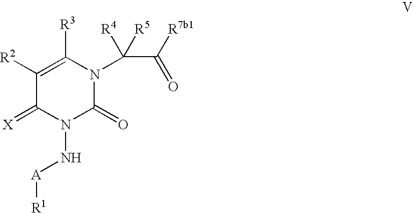
wherein
R^{7b1} is H or methyl;
X represents O or S;
A represents C(O), S(O)₂, C(O)O (in which latter group the O moiety is attached to R¹), C(O)NH, S(O)₂NH (in which latter two groups the NH moiety is attached to R¹), a direct bond or C₁₋₁₆ alkylene (which latter group is optionally substituted, at the C-atom to which the NH moiety is attached by C(O)OR^{A} or C(O)N(H)R^{A});
R^{A} represents H or C₁₋₄ alkyl;
R¹ represents
(a) C₁₋₁₀ alkyl, C₂₋₁₀ alkenyl, C₂₋₁₀ alkynyl (which latter three groups are optionally substituted by one or more substituents selected from halo, CN, C₃₋₁₀ cycloalkyl (optionally substituted by one or more substituents selected from halo, OH, ═O, C₁₋₆ alkyl, C₁₋₆ alkoxy and aryl), OR^{6a}, S(O)ₙR^{6b}, S(O)₂N(R^{6c})(R^{6d}), N(R^{6e})S(O)₂R^{6f}, N(R^{6g})(R^{6h}), B¹—C(O)—B²—R⁶ⁱ, aryl and Het¹),
(b) C₃₋₁₀ cycloalkyl or C₄₋₁₀ cycloalkenyl, which latter two groups are optionally substituted by one or more substituents selected from halo, ═O, CN, C₁₋₁₀ alkyl, C₃₋₁₀ cycloalkyl (optionally substituted by one or more substituents selected from halo, OH, ═O, C₁₋₆ alkyl, C₁₋₆ alkoxy and aryl), OR^{6a}, S(O)ₙR^{6b}, S(O)₂N(R^{6c})(R^{6d}), N(R^{6e})S(O)₂R^{6f}, N(R^{6g})(R^{6h}), B³—C(O)—B⁴—R⁶ⁱ, aryl and Het²,
(c) aryl, or
(d) Het³;
R^{6a} to R⁶ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, aryl and Het⁴),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, aryl and Het⁵),
(d) aryl or
(e) Het⁶,
provided that R^{6b} does not represent H when n is 1 or 2;
R² represents H or halo;
R³ represents
(a) H,
(b) halo,
(c) CN,
(d) C₁₋₆ alkyl, C₂₋₆ alkenyl, C₂₋₆ alkynyl, C₁₋₆ alkoxy (which latter four groups are optionally substituted by one or more substituents selected from halo, OH, CN, C₁₋₄ alkoxy, C(O)OH, C(O)O—C₁₋₄ alkyl and OC(O)—C₁₋₄ alkyl) or
(e) together with R⁴, R³ represents C₂₋₃ n-alkylene, T¹-(C₁₋₂ n-alkylene) or (C₁₋₂ n-alkylene)-T¹, which latter three groups are optionally substituted by halo, or
(f) together with R⁴ and R⁵, R³ represents T²-[C(H)═], wherein T² is bonded to the C-atom to which the group R³ is attached;
R⁴ and R⁵ independently represent H, F or methyl (which latter group is optionally substituted by one or more F atoms) or
(a) together with R³, R⁴ represents C₂₋₃ n-alkylene, T¹-(C₁₋₂ n-alkylene) or (C₁₋₂ n-alkylene)-T¹, which latter three groups are optionally substituted by halo, or
(b) together with R³, R⁴ and R⁵ represent T²-[C(H)═], wherein T² is bonded to the C-atom to which the group R³ is attached;
T¹ and T² independently represent O, S, or NR⁷;
R⁷ represents H or C₁₋₄ alkyl
n, p, and q, independently represent 0, 1 or 2;
each aryl independently represents a C₆₋₁₀ carbocyclic aromatic group, which group may comprise either one or two rings and may be substituted by one or more substituents selected from
(a) halo,
(b) CN,
(c) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, C(O)OH, C(O)O—C₁₋₆ alkyl, phenyl (which latter group is optionally substituted by halo) and Het⁷),
(d) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het⁸),
(e) OR^{17a},
(f) S(O)ₚR^{17b},
(g) S(O)₂N(R^{17c})(R^{17d}),
(h) N(R^{17e})S(O)₂R^{17f},
(i) N(R^{17g})(R^{17h}),
(j) B⁵—C(O)—B⁶—R¹⁷ⁱ,
(k) phenyl (which latter group is optionally substituted by halo),
(l) Het⁹ and
(m) Si(R^{18a})(R^{18b})(R^{18c}),
R^{17a} to R¹⁷ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, phenyl (which latter group is optionally substituted by halo) and Het¹⁰),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het¹¹),
(d) phenyl (which latter group is optionally substituted by halo) or
(e) Het¹²,
provided that R^{17b} does not represent H when p is 1 or 2;
B¹ to B⁸ independently represent a direct bond, O, S, NH or N—C₁₋₄ alkyl;
Het¹ to Het¹² independently represent 4- to 14-membered heterocyclic groups containing one or more heteroatoms selected from oxygen, nitrogen and/or sulfur, which heterocyclic groups may comprise one, two or three rings and may be substituted by one or more substituents selected from
(a) halo,
(b) CN,
(c) C₁₋₁₀ alkyl, C₂₋₁₀ alkenyl, C₂₋₁₀ alkynyl (which latter four groups are optionally substituted by one or more substituents selected from halo, OH, C₁₋₆ alkoxy, C(O)OH, C(O)O—C₁₋₆ alkyl, phenyl (which latter group is optionally substituted by halo) and Het^{a}),
(d) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het^{b}),
(e) ═O,
(f) OR^{19a},
(g) S(O)_{q}R^{19b},
(h) S(O)₂N(R^{19c})(R^{19d}),
(i) N(R^{19e})S(O)₂R^{19f},
(j) N(R^{19g})(R^{19h}, )
(k) B⁷—C(O)—B⁸—R¹⁹ⁱ,
(l) phenyl (which latter group is optionally substituted by halo),
(m) Het^{c} and
(n) Si(R^{20a})(R^{20b})(R^{20c});
R^{19a} to R¹⁹ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, phenyl (which latter group is optionally substituted by halo) and Het^{d}),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het^{e}),
(d) phenyl (which latter group is optionally substituted by halo) or
(e) Het^{f},
provided that R^{19b} does not represent H when q is 1 or 2;
Het^{a} to Het^{f} independently represent 5- or 6-membered heterocyclic groups containing one to four heteroatoms selected from oxygen, nitrogen and/or sulfur, which heterocyclic groups may be substituted by one or more substituents selected from halo, ═O and C₁₋₆ alkyl;
R^{18a}, R^{18b}, R^{18c}, R^{20a}, R^{20b} and R^{20c} independently represent C₁₋₆ alkyl or phenyl (which latter group is optionally substituted by halo or C₁₋₄ alkyl;
unless otherwise specified
(i) alkyl, alkenyl, alkynyl, cycloalkyl, cycloalkenyl, alkylene and alkenylene groups, as well as the alkyl part of alkoxy groups, may be substituted by one or more halo atoms, and
(ii) cycloalkyl and cycloalkenyl groups may comprise one or two rings and may additionally be ring-fused to one or two phenyl groups;
or a protected derivative thereof.

13. A compound of formula VI, 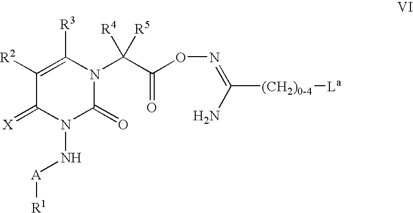
wherein
L^{a} represents
(a) C₀₋₆ alkylene-R^{a}, except that it does not represent C₀ alkylene-R^{a},
(b) C₀₋₂ alkylene-CH═CH—C₀₋₂ alkylene-R^{a},
(c) C₀₋₂ alkylene-C≡C—C₀₋₂ alkylene-R^{a}, 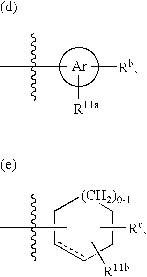
wherein the dashed line represents an optional double bond, or 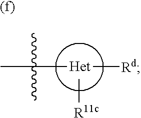
Ar represents phenyl or naphthyl;
Het represents a 5- to 10-membered heterocyclic group comprising one or two rings and containing, as heteroatom(s), one sulfur or oxygen atom and/or one or more nitrogen atoms;
R^{11a} represents H or one or more substituents selected from halo, OH, CN, C₁₋₆ alkyl, C₁₋₆ alkoxy (which latter two groups are optionally substituted by one or more substituents selected from halo, OH, C₁₋₄ alkoxy, C(O)OR^{12a} and C(O)N(R^{12b})R^{12c} and S(O)₀₋₂R^{12d};
R^{11b} and R^{11c} independently represent H or one or more substituents selected from halo, OH, CN, C₁₋₆ alkyl, C₁₋₆ alkoxy (which latter two groups are optionally substituted by one or more substituents selected from halo, OH, C₁₋₄ alkoxy, C(O)OR^{12a} and C(O)N(R^{12b})R^{12c}, S(O)₀₋₂R^{12d}, ═O, ═NH, ═NOH and ═N—CN;
R^{12a} to R^{12c} independently represent H, C₁₋₆ alkyl or C₃₋₇ cycloalkyl (which latter two groups are optionally substituted by one OH or N(R^{12e})R^{12f} group or by one or more halo atoms);
R^{12d} represents independently at each occurrence, C₁₋₆ alkyl optionally substituted by one OH or N(R^{12e})R^{12f} group or by one or more halo atoms;
R^{12e} and R^{12f} represent, independently at each occurrence, H or C₁₋₄ alkyl optionally substituted by one or more halo atoms;
R^{a} to R^{d} independently represent 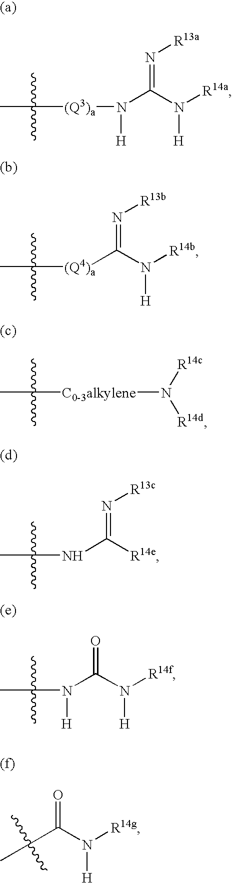
(g) Het^{x}
or R^{b} to R^{d} may also represent H;
Q³ represents O, N(R^{10c}), S(O)₂, S(O)₂NH, C(O) or —CH═N—;
Q⁴ represents O, S or CH₂;
a represents 0 or 1;
Het^{x} represents a 5- or 6-membered heterocyclic group containing one to four heteroatoms selected from oxygen, nitrogen and/or sulfur, which heterocyclic group may be substituted by one or more substituents selected from halo, ═O, C₁₋₆ alkyl and C₁₋₆ alkoxy (which latter two groups are optionally substituted by one or more halo atoms);
R^{13a} to R^{13c} independently represent
(a) H,
(b) CN,
(c) NH₂,
(d) OR¹⁵ or
(e) C(O)OR¹⁶;
R¹⁵ represents
(a) H,
(b) C₁₋₁₀ alkyl, C₃₋₁₀ alkenyl, or C₃₋₁₀ alkynyl,
(c) C₃₋₁₀ cycloalkyl, C₄₋₁₀ cycloalkenyl, which latter two groups are optionally substituted by one or more substituents selected from halo and C₁₋₆ alkyl, or
(d) C₁₋₃ alkyl, which latter group is optionally interrupted by oxygen and is substituted by aryl or —O-aryl;
R¹⁶ represents
(a) C₁₋₁₀ alkyl, C₃₋₁₀ alkenyl, C₃₋₁₀ alkynyl, which latter three groups are optionally interrupted by one or more oxygen atoms, or
(b)C₃₋₁₀ cycloalkyl, C₄₋₁₀ cycloalkenyl, which latter two groups are optionally substituted by one or more substituents selected from halo and C₁₋₆ alkyl, or
(c) C₁₋₃ alkyl, which latter group is optionally interrupted by oxygen and is substituted by aryl or —O-aryl;
R^{10c} and R^{14a} to R^{14g} independently represent
(a) H or
(b) C₁₋₄ alkyl (which latter group is optionally substituted by one or more substituents selected from halo and OH),
or R^{14a} and R^{14b} independently represent C(O)O—C₁₋₆ alkyl (the alkyl part of which latter group is optionally substituted by aryl and/or one or more halo atoms),
or R^{14c} represents
(a) C₁₋₄ alkyl substituted by C₃₋₇ cycloalkyl or aryl,
(b) C₃₋₇ cycloalkyl,
(c) C(O)O—C₁₋₆ alkyl (the alkyl part of which latter group is optionally substituted by aryl and/or one or more halo atoms),
(d) C(O)C₁₋₆ alkyl,
(e) C(O)N(H)—C₁₋₆ alkyl (the alkyl part of which latter group is optionally substituted by aryl and/or one or more halo atoms) or
(f) S(O)₂—C₁₋₆ alkyl (the alkyl part of which latter group is optionally substituted by aryl and/or one or more halo atoms),
or R^{14c} and R^{14d} together represent C₃₋₆ n-alkylene optionally interrupted by O, S, N(H) or N(C₁₋₄ alkyl) and/or substituted by one or more C₁₋₄ alkyl groups;
X represents O or S;
A represents C(O), S(O)₂, C(O)O (in which latter group the O moiety is attached to R¹), C(O)NH, S(O)₂NH (in which latter two groups the NH moiety is attached to R¹), a direct bond or C₁₋₆ alkylene (which latter group is optionally substituted, at the C-atom to which the NH moiety is attached, by C(O)OR^{A} or C(O)N(H)R^{A});
R^{A} represents H or C₁₋₄ alkyl;
R¹ represents
(a) C₁₋₁₀ alkyl, C₂₋₁₀ alkenyl, C₂₋₁₀ alkynyl (which latter three groups are optionally substituted by one or more substituents selected from halo, CN, C₃₋₁₀ cycloalkyl (optionally substituted by one or more substituents selected from halo, OH, ═O, C₁₋₆ alkyl, C₁₋₆ alkoxy and aryl) OR^{6a}, S(O)ₙR^{6b}, S(O)₂N(R^{6c})(R^{6d}), N(R^{6e})S(O)₂R^{6f}, N(R^{6g})(R^{6h}), B¹—C(O)—B²—R⁶ⁱ, aryl and Het¹),
(b) C₃₋₁₀ cycloalkyl or C₄₋₁₀ cycloalkenyl, which latter two groups are optionally substituted by one or more substituents selected from halo, ═O, CN, C₁₋₁₀ alkyl, C₃₋₁₀ cycloalkyl (optionally substituted by one or more substituents selected from halo, OH, ═O, C₁₋₆ alkyl, C₁₋₆ alkoxy and aryl), OR^{6a}, S(O)ₙR^{6b}, S(O)₂N(R^{6c})(R^{6d}), N(R^{6e})S(O)₂R^{6f}, N(R^{6g})(R^{6h}), B³—C(O)—B⁴—R⁶ⁱ, aryl and Het²,
(c) aryl, or
(d) Het³;
R^{6a} to R⁶ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, aryl and Het⁴),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, aryl and Het⁵),
(d) aryl or
(e) Het⁶,
provided that R^{6b} does not represent H when n is 1 or 2;
R² represents H or halo;
R³ represents
(a) H,
(b) halo,
(c) CN,
(d) C₁₋₆ alkyl, C₂₋₆ alkenyl, C₂₋₆ alkynyl, C₁₋₆ alkoxy (which latter four groups are optionally substituted by one or more substituents selected from halo, OH, CN, C₁₋₄ alkoxy, C(O)OH, C(O)O—C₁₋₄ alkyl and OC(O)—C₁₋₄ alkyl) or
(e) together with R⁴, R³ represents C₂₋₃ n-alkylene, T¹-(C₁₋₂ n-alkylene) or (C₁₋₂ n-alkylene)-T¹, which latter three groups are optionally substituted by halo, or
(f) together with R⁴ and R⁵, R³ represents T²-[C(H)═], wherein T² is bonded to the C-atom to which the group R³ is attached;
R⁴ and R⁵ independently represent H, F or methyl (which latter group is optionally substituted by one or more F atoms), or
(a) together with R³, R⁴ represents C₂₋₃ n-alkylene, T¹-(C₁₋₂ n-alkylene) or (C₁₋₂ n-alkylene)-T¹, which latter three groups are optionally substituted by halo, or
(b) together with R³, R⁴ and R⁵ represent T²-[C(H)═], wherein T² is bonded to the C-atom to which the group R³ is attached;
T¹ and T² independently represent O, S, or NR⁷;
R⁷ represents H or C₁₋₄ alkyl;
n, p, and g, independently, represent 0, 1 or 2;
each aryl independently represents a C₆₋₁₀ carbocyclic aromatic group, which group may comprise either one or two rings and may be substituted by one or more substituents selected from
(a) halo,
(b) CN,
(c) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, C(O)OH, C(O)O—C₁₋₆ alkyl, phenyl (which latter group is optionally substituted by halo) and Het⁷),
(d) C₁₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het⁸),
(e) OR^{17a},
(f) S(O)ₚR^{17b},
(g) S(O)₂N(R^{17c})(R^{17d}),
(h) N(R^{17e})S(O)₂R^{17f},
(i) N(R^{17g})(R^{17h}),
(j) B⁵—C(O)—B⁶—R¹⁷ⁱ,
(k) phenyl (which latter group is optionally substituted by halo),
(l) Het⁹ and
(m) Si(R^{18a})(R^{18b})(R^{18c}),
R^{17a} to R¹⁷ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, phenyl (which latter group is optionally substituted by halo) and Het¹⁰),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het¹¹),
(d) phenyl (which latter group is optionally substituted by halo) or
(e) Het¹²,
provided that R^{17b} does not represent H when p is 1 or 2;
B¹ to B⁸ independently represent a direct bond, O, S, NH or N—C₁₋₄ alkyl;
Het¹ to Het¹² independently represent 4- to 14-membered heterocyclic groups containing one or more heteroatoms selected from oxygen, nitrogen and/or sulfur, which heterocyclic groups may comprise one, two or three rings and may be substituted by one or more substituents selected from
(a) halo,
(b) CN,
(c) C₁₋₁₀ alkyl, C₂₋₁₀ alkenyl, C₂₋₁₀ alkynyl (which latter four groups are optionally substituted by one or more substituents selected from halo, OH, C₁₋₆ alkoxy, C(O)OH, C(O)O—C₁₋₆ alkyl, phenyl (which latter group is optionally substituted by halo) and Het^{a}),
(d) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het^{b}),
(e) ═O,
(f) OR^{19a},
(g) S(O)_{q}R^{19b},
(h) S(O)₂N(R^{19c})(R^{19d}),
(i) N(R^{19e})S(O)₂R^{19f},
(j) N(R^{19g})(R^{19h}),
(k) B⁷—C(O)—B⁸—R¹⁹ⁱ,
(l) phenyl (which latter group is optionally substituted by halo),
(m) Het^{c} and
(n) Si(R^{20a})(R^{20b})(R^{20c});
R^{19a} to R¹⁹ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, phenyl (which latter group is optionally substituted by halo) and Het^{d}),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het^{e}),
(d) phenyl (which latter group is optionally substituted by halo) or
(e) Het^{f},
provided that R^{19b} does not represent H when q is 1 or 2;
Het^{a} to Het^{f} independently represent 5- or 6-membered heterocyclic groups containing one to four heteroatoms selected from oxygen, nitrogen and/or sulfur, which heterocyclic groups may be substituted by one or more substituents selected from halo, ═O and C₁₋₆ alkyl;
R^{18a}, R^{18b}, R^{18c}, R^{20a}, R^{20b} and R^{20c} independently represent C₁₋₆ alkyl or phenyl (which latter group is optionally substituted by halo or C₁₋₄ alkyl);
unless otherwise specified
(i) alkyl, alkenyl, alkynyl, cycloalkyl, cycloalkenyl, alkylene and alkenylene groups as well as the alkyl part of alkoxy groups, may be substituted by one or more halo atoms, and
(ii) cycloalkyl and cycloalkenyl groups may comprise one or two rings and may additionally be ring-fused to one or two phenyl groups;
or a protected derivative thereof.

14. A compound of formula VII, 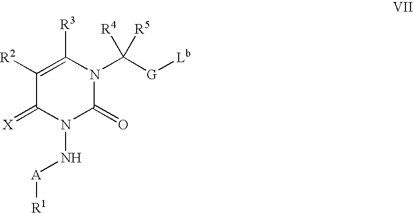
wherein
L^{b} represents
(a) C₀₋₆ alkylene-R^{a},
(b) C₀₋₂ alkylene-CH═CH—C₀₋₂ alkylene-R^{a},
(c) C₀₋₂ alkylene-C≡C—C₀₋₂ alkylene-R^{a}, 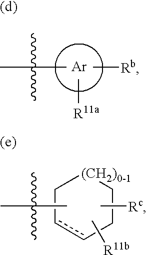
wherein the dashed line represents an optional double bond, or 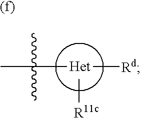
Ar represents phenyl or naphthyl;
Het represents a 5- to 10-membered heterocyclic group comprising one or two rings and containing, as heteroatom(s), one sulfur or oxygen atom and/or one or more nitrogen atoms;
R^{11a} represents H or one or more substituents selected from halo, OH, CN, C₁₋₆ alkyl, C₁₋₆ alkoxy (which latter two groups are optionally substituted by one or more substituents selected from halo, OH, C₁₋₄ alkoxy, C(O)OR^{12a} and C(O)N(R^{12b})R^{12c} and S(O)₀₋₂R^{12d};
R^{11b} and R^{11c} independently represent H or one or more substituents selected from halo, OH, CN, C₁₋₆ alkyl, C₁₋₆ alkoxy (which latter two groups are optionally substituted by one or more substituents selected from halo, OH, C₁₋₄ alkoxy, C(O)OR^{12a} and C(O)N(R^{12b})R^{12c}, S(O)₀₋₂R^{12d}, ═O, ═NH, ═NOH and ═N—CN;
R^{12a} to R^{12c} independently represent H, C₁₋₆ alkyl or C₃₋₇ cycloalkyl (which latter two groups are optionally substituted by one OH or N(R^{12e})R^{12f} group or by one or more halo atoms);
R^{12d} represents, independently at each occurrence, C₁₋₆ alkyl optionally substituted by one OH or N(R^{12e})R^{12f} group or by one or more halo atoms;
R^{12e} and R^{12f} represent, independently at each occurrence, H or C₁₋₄ alkyl optionally substituted by one or more halo atoms;
R^{a} to R^{d} independently represent a cyano or —C(═NH)O—C₁₋₄ alkyl group,
G represents
(a) —C(R^{7a})(R^{7b})N(R^{8a})—[CH(C(O)R⁹)]₀₋₁—C₀₋₃ alkylene-(Q¹)ₐ-,
(b) —C(R^{7a})(R^{7b})(O)N(R^{8b})—C₂₋₃ alkenylene-(Q¹)ₐ-, 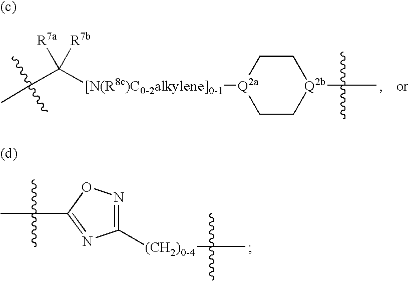
R^{7a} and R^{7b} independently represent H or methyl, or R^{7a} and R^{7b} together represent ═O;
R⁹ represents H or a 5- to 10-membered aromatic heterocyclic group comprising one or two rings and containing, as heteroatom(s), one sulfur or oxygen atom and/or one or more nitrogen atoms, which heterocyclic group is optionally substituted by one or more substituents selected from halo and C₁₋₆ alkyl;
Q¹ represents O, NR^{10a}, [N(H)]₀₋₁C(O)—C₀₋₂ alkylene, C(O)NHNHC(O), or —N═C(R^{10b})—;
a represents 0 or 1;
Q^{2a} represents 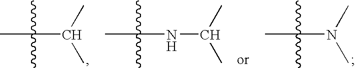
Q^{2b} represents 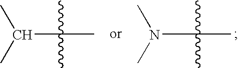
R^{8a} to R^{8c}, R^{10a}, and R^{11b} independently represent
(a) H or
(b) C₁₋₄ alkyl (which latter group is optionally substituted by one or more substituents selected from halo and OH),
X represents O or S;
A represents C(O), S(O)₂, C(O)O (in which latter group the O moiety is attached to R¹), C(O)NH, S(O)₂NH (in which latter two groups the NH moiety is attached to R¹), a direct bond or C₁₋₆ alkylene (which latter group is optionally substituted, at the C-atom to which the NH moiety is attached, by C(O)OR^{A} or C(O)N(H)R^{A});
R^{A} represents H or C₁₋₄ alkyl;
R¹ represents
(a) C₁₋₁₀ alkyl, C₂₋₁₀ alkenyl, C₂₋₁₀ alkynyl (which latter three groups are optionally substituted by one or more substituents selected from halo, CN, C₃₋₁₀ cycloalkyl (optionally substituted by one or more substituents selected from halo, OH, ═O, C₁₋₆ alkyl, C₁₋₆ alkoxy and aryl), OR^{6a}, S(O)ₙR^{6b}, S(O)₂N(R^{6c})(R^{6d}), N(R^{6e})S(O)₂R^{6f}, N(R^{6g})(R^{6h}), B¹—C(O)—B²—R⁶ⁱ, aryl and Het¹),
(b)C₃₋₁₀ cycloalkyl or C₄₋₁₀ cycloalkenyl, which latter two groups are optionally substituted by one or more substituents selected from halo, ═O, CN, C₁₋₁₀ alkyl, C₃₋₁₀ cycloalkyl (optionally substituted by one or more substituents selected from halo, OH, ═O, C₁₋₆ alkyl, C₁₋₆ alkoxy and aryl), OR^{6a}, S(O)ₙR^{6b}, S(O)₂N(R^{6c})(R^{6d}), N(R^{6e})S(O)₂R^{6f}, N(R^{6g})(R^{6h}), B³—C(O)—B⁴—R⁶ⁱ, aryl and Het²,
(c) aryl, or
(d) Het³;
R^{6a} to R⁶ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, aryl and Het⁴),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, aryl and Het⁵),
(d) aryl or
(e) Het⁶,
provided that R^{6b} does not represent H when n is 1 or 2;
R² represents H or halo;
R³ represents
(a) H,
(b) halo,
(c) CN,
(d) C₁₋₆ alkyl, C₂₋₆ alkenyl, C₂₋₆ alkynyl, C₁₋₆ alkoxy (which latter four groups are optionally substituted by one or more substituents selected from halo, OH, CN, C₁₋₄ alkoxy, C(O)OH, C(O)O—C₁₋₄ alkyl and OC(O)—C₁₋₄ alkyl) or
(e) together with R⁴, R³ represents C₂₋₃ n-alkylene, T¹-(C₁₋₂ n-alkylene) or (C₁₋₂ n-alkylene)-T¹, which latter three groups are optionally substituted by halo, or
(f) together with R⁴ and R⁵, R³ represents T²-[C(H)═], wherein T² is bonded to the C-atom to which the group R³ is attached;
R⁴ and R⁵ independently represent H, F or methyl (which latter group is optionally substituted by one or more F atoms), or
(a) together with R³, R⁴ represents C₂₋₃ n-alkylene, T¹-(C₁₋₂ n-alkylene) or (C₁₋₂ n-alkylene)-T¹, which latter three groups are optionally substituted by halo, or
(b) together with R³, R⁴ and R⁵ represent T²-[C(H)═], wherein T² is bonded to the C-atom to which the group R³ is attached;
T¹ and T² independently represent O, S, or NR⁷;
R⁷ represents H or C₁₋₄ alkyl;
n, p, and q, independently represent 0, 1 or 2;
each aryl independently represents a C₆₋₁₀ carbocyclic aromatic group, which group may comprise either one or two rings and may be substituted by one or more substituents selected from
(a) halo,
(b) CN,
(c) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, C(O)OH, C(O)O—C₁₋₆ alkyl, phenyl (which latter group is optionally substituted by halo) and Het⁷),
(d) C₃₋₁₀ cycloalkyl C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het⁸),
(e) OR^{17a},
(f) S(O)ₚR^{17b},
(g) S(O)₂N(R^{17c})(R^{17d}),
(h) N(R^{17e})S(O)₂R^{17f},
(i) N(R^{17g})(R^{17h}),
(j) B⁵—C(O)—B⁶—R¹⁷ⁱ,
(k) phenyl (which latter group is optionally substituted by halo),
(l) Het⁹ and
(m) Si(R^{18a})(R^{18b})(R^{18c}),
R^{17a} to R¹⁷ⁱ independently represent at each occurrence
(a) H,
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, phenyl (which latter group is optionally substituted by halo) and Het¹⁰),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het¹¹),
(d) phenyl (which latter group is optionally substituted by halo) or
(e) Het¹²,
provided that R^{17b} does not represent H when p is 1 or 2;
B¹ to B⁸ independently represent a direct bond, O, S, NH or N—C₁₋₄ alkyl;
Het¹ to Het¹² independently represent 4- to 14-membered heterocyclic groups containing one or more heteroatoms selected from oxygen, nitrogen and/or sulfur, which heterocyclic groups may comprise one, two or three rings and may be substituted by one or more substituents selected from
(a) halo,
(b) CN,
(c) C₁₋₁₀ alkyl, C₂₋₁₀ alkenyl, C₂₋₁₀ alkynyl (which latter four groups are optionally substituted by one or more substituents selected from halo, OH, C₁₋₆ alkoxy, C(O)OH, C(O)O—C₁₋₆ alkyl, phenyl (which latter group is optionally substituted by halo) and Het^{a}),
(d) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het^{b}),
(e) ═O,
(f) OR^{19a},
(g) S(O)_{q}R^{19b},
(h) S(O)₂N(R^{19c})(R^{19d}),
(i) N(R^{19e})S(O)₂R^{19f},
(j) N(R^{19g})(R^{19h}),
(k) B⁷—C(O)—B⁸—R¹⁹ⁱ,
(l) phenyl (which latter group is optionally substituted by halo),
(m) Het^{c} and
(n) Si(R^{20a})(R^{20b})(R^{20c});
R^{19a} to R¹⁹ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, phenyl (which latter group is optionally substituted by halo) and Het^{d}),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het^{e}),
(d) phenyl (which latter group is optionally substituted by halo) or
(e) Het^{f},
provided that R^{19b} does not represent H when q is 1 or 2;
Het^{a} to Het^{f} independently represent 5- or 6-membered heterocyclic groups containing one to four heteroatoms selected from oxygen, nitrogen and/or sulfur, which heterocyclic groups may be substituted by one or more substituents selected from halo, ═O and C₁₋₆ alkyl
R^{18a}, R^{18b}, R^{18c}, R^{20a}, R^{20b} and R^{20c} independently represent C₁₋₆ alkyl or phenyl (which latter group is optionally substituted by halo or C₁₋₄ alkyl;
unless otherwise specified
(i) alkyl, alkenyl, alkynyl, cycloalkyl, cycloalkenyl, alkylene and alkenylene groups, as well as the alkyl part of alkoxy groups, may be substituted by one or more halo atoms, and
(ii) cycloalkyl and cycloalkenyl groups may comprise one or two rings and may additionally be ring-fused to one or two phenyl groups;
or a protected derivative thereof.

15. A compound of formula VIII, 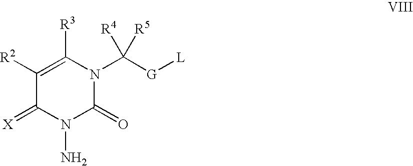
G represents
(a) —C(R^{7a})(R^{7b})N(R^{8a})—[CH(C(O)R⁹)]₀₋₁—C₀₋₃ alkylene-(Q¹)ₐ-,
(b) —C(R^{7a})(R^{7b})(O)N(R^{8b})—C₂₋₃ alkenylene-(Q¹)ₐ-, 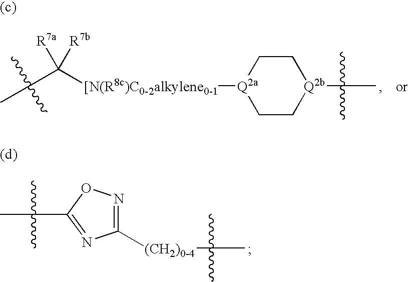
R^{7a} and R^{7b} independently represent H or methyl, or R^{7a} and R^{7b} together represent ═O;
R⁹ represents H or a 5- to 10-membered aromatic heterocyclic group comprising one or two rings and containing, as heteroatom(s), one sulfur or oxygen atom and/or one or more nitrogen atoms, which heterocyclic group is optionally substituted by one or more substituents selected from halo and C₁₋₆ alkyl;
Q¹ represents O, NR^{10a}, [N(H)]₀₋₁C(O)—C₀₋₂ alkylene, C(O)NHNHC(O), or —N═C(R^{10b})—;
a represents 0 or 1;
Q^{2a} represents 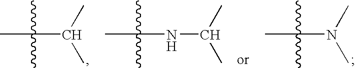
Q^{2b} represents 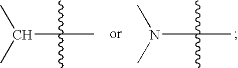
R^{8a} to R^{8c}, R^{10a} and R^{10b} independently represent
(a) H or
(b) C₁₋₄ alkyl (which latter group is optionally substituted by one or more substituents selected from halo and OH),
L represents
(a) C₀₋₆ alkylene-R^{a},
(b) C₀₋₂ alkylene-CH═CH—C₀₋₂ alkylene-R^{a},
(c) C₀₋₂ alkylene-C≡C—C₀₋₂ alkylene-R^{a}, 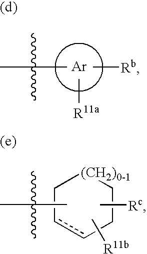
wherein the dashed line represents an optional double bond, or 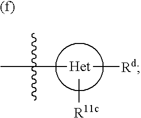
Ar represents phenyl or naphthyl;
Het represents a 5- to 10-membered heterocyclic group comprising one or two rings and containing, as heteroatom(s), one sulfur or oxygen atom and/or one or more nitrogen atoms;
R^{11a} represents H or one or more substituents selected from halo, OH, CN, C₁₋₆ alkyl, C₁₋₆ alkoxy (which latter two groups are optionally substituted by one or more substituents selected from halo, OH, C₁₋₄ alkoxy, C(O)OR^{12a} and C(O)N(R^{12b})R^{12c} and S(O)₀₋₂R^{12d};
R^{11b} and R^{11c} independently represent H or one or more substituents selected from halo, OH, CN, C₁₋₆ alkyl, C₁₋₆ alkoxy (which latter two groups are optionally substituted by one or more substituents selected from halo, OH, C₁₋₄ alkoxy, C(O)OR^{12a} and C(O)N(R^{12b})R^{12c}, S(O)₀₋₂R^{12d}, ═O, ═NH, ═NOH and ═N—CN;
R^{12a} to R^{12c} independently represent H, C₁₋₆ alkyl or C₃₋₇ cycloalkyl (which latter two groups are optionally substituted by one OH or N(R^{12e})R^{12f} group or by one or more halo atoms); R^{12d} represents, independently at each occurrence, C₁₋₆ alkyl optionally substituted by one OH or N(R^{12e})R^{12f} group or by one or more halo atoms;
R^{12e} and R^{12f} represent, independently at each occurrence, H or C₁₋₄ alkyl optionally substituted by one or more halo atoms;
R^{a} to R^{d} independently represent 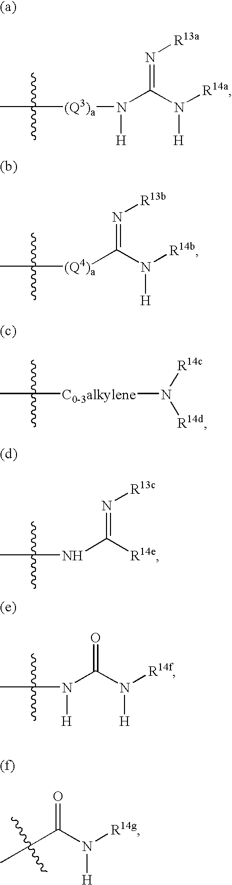
(g) Het^{x}
or R^{b} to R^{d} may also represent H;
Q³ represents O, N(R^{10c}), S(O)₂, S(O)₂NH, C(O) or —CH═N—;
Q⁴ represents O, S or CH₂;
Het^{x} represents a 5- or 6-membered heterocyclic group containing one to four heteroatoms selected from oxygen, nitrogen and/or sulfur, which heterocyclic group may be substituted by one or more substituents selected from halo, ═O, C₁₋₆ alkyl and C₁₋₆ alkoxy (which latter two groups are optionally substituted by one or more halo atoms);
R^{13a} to R^{13c} independently represent
(a) H,
(b) CN,
(c) NH₂,
(d) OR¹⁵ or
(e) C(O)OR¹⁶;
R¹⁵ represents
(a) H,
(b) C₁₋₁₀ alkyl, C₃₋₁₀ alkenyl, or C₃₋₁₀ alkynyl,
(c) C₃₋₁₀ cycloalkyl, C₄₋₁₀ cycloalkenyl, which latter two groups are optionally substituted by one or more substituents selected from halo and C₁₋₆ alkyl, or
(d) C₁₋₃ alkyl, which latter group is optionally interrupted by oxygen and is substituted by aryl or —O-aryl;
R¹⁶ represents
(a) C₁₋₁₀ alkyl, C₃₋₁₀ alkenyl, C₃₋₁₀ alkynyl, which latter three groups are optionally interrupted by one or more oxygen atoms, or
(b) C₃₋₁₀ cycloalkyl, C₄₋₁₀ cycloalkenyl, which latter two groups are optionally substituted by one or more substituents selected from halo and C₁₋₆ alkyl, or
(c) C₁₋₃ alkyl, which latter group is optionally interrupted by oxygen and is substituted by aryl or —O-aryl;
R^{10c} and R^{14a} to R^{14g} independently represent
(a) H or
(b) C₁₋₄ alkyl (which latter group is optionally substituted by one or more substituents selected from halo and OH),
or R^{14a} and R^{14b} independently represent C(O)O—C₁₋₆ alkyl (the alkyl part of which latter group is optionally substituted by aryl and/or one or more halo atoms),
or R^{14c} represents
(a) C₁₋₄ alkyl substituted by C₃₋₇ cycloalkyl or aryl,
(b) C₁₋₄ cycloalkyl,
(c) C(O)O—C₁₋₆ alkyl (the alkyl part of which latter group is optionally substituted by aryl and/or one or more halo atoms),
(d) C(O)C₁₋₆ alkyl,
(e) C(O)N(H)—C₁₋₆ alkyl (the alkyl part of which latter group is optionally substituted by aryl and/or one or more halo atoms) or
(f) S(O)₂—C₁₋₆ alkyl (the alkyl part of which latter group is optionally substituted by aryl and/or one or more halo atoms),
or R^{14c} and R^{14d} together represent C₃₋₆ n-alkylene optionally interrupted by O, S, N(H) or N(C₁₋₄ alkyl) and/or substituted by one or more C₁₋₄ alkyl groups;
X represents O or S;
R² represents H or halo;
R³ represents
(a) H,
(b) halo,
(c) CN,
(d) C₁₋₆ alkyl, C₂₋₆ alkenyl, C₂₋₆ alkynyl, C₁₋₆ alkoxy (which latter four groups are optionally substituted by one or more substituents selected from halo, OH, CN, C₁₋₄ alkoxy, C(O)OH, C(O)O—C₁₋₄ alkyl and OC(O)—C₁₋₄ alkyl) or
(e) together with R⁴, R³ represents C₂₋₃ n-alkylene, T¹-(C₁₋₂ n-alkylene) or (C₁₋₂ n-alkylene)-T¹, which latter three groups are optionally substituted by halo, or
(f) together with R⁴ and R⁵, R³ represents T²-[C(H)═], wherein T² is bonded to the C-atom to which the group R³ is attached;
R⁴ and R⁵ independently represent H, F or methyl (which latter group is optionally substituted by one or more F atoms), or
(a) together with R³, R⁴ represents C₂₋₃ n-alkylene, T¹-(C₁₋₂ n-alkylene) or (C₁₋₂ n-alkylene)-T¹, which latter three groups are optionally substituted by halo, or
(b) together with R³, R⁴ and R⁵ represent T²-[C(H)═], wherein T² is bonded to the C-atom to which the group R³ is attached;
T¹ and T² independently represent O, S, or NR⁷;
R⁷ represents H or C₁₋₄ alkyl;
p and q, independently, represent 0, 1 or 2;
each aryl independently represents a C₆₋₁₀ carbocyclic aromatic group, which group may comprise either one or two rings and may be substituted by one or more substituents selected from
(a) halo,
(b) CN,
(c) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, C(O)OH, C(O)O—C₁₋₆ alkyl, phenyl (which latter group is optionally substituted by halo) and Het⁷),
(d) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het⁸),
(e) OR^{17a},
(f) S(O)ₚR^{17b},
(g) S(O)₂N(R^{17c})(R^{17d}),
(h) N(R^{17e})S(O)₂R^{17f},
(i) N(R^{17g})(R^{17h}),
(j) B⁵—C(O)—B⁶—R¹⁷ⁱ,
(k) phenyl (which latter group is optionally substituted by halo),
(l) Het⁹ and
(m) Si(R^{18a})(R^{18b})(R^{18c}),
R^{17a} to R¹⁷ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, phenyl (which latter group is optionally substituted by halo) and Het¹⁰),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het¹¹),
(d) phenyl (which latter group is optionally substituted by halo) or
(e) Het¹²,
provided that R^{17b} does not represent H when p is 1 or 2;
B⁵ to B⁸ independently represent a direct bond, O, S, NH or N—C₁₋₄ alkyl;
Het⁷ to Het¹² independently represent 4- to 14-membered heterocyclic groups containing one or more heteroatoms selected from oxygen, nitrogen and/or sulfur, which heterocyclic groups may comprise one, two or three rings and may be substituted by one or more substituents selected from
(a) halo,
(b) CN,
(c) C₁₋₁₀ alkyl, C₂₋₁₀ alkenyl, C₂₋₁₀ alkynyl (which latter four groups are optionally substituted by one or more substituents selected from halo, OH, C₁₋₆ alkoxy, C(O)OH, C(O)O—C₁₋₆ alkyl, phenyl (which latter group is optionally substituted by halo) and Het^{a}),
(d) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het^{b}),
(e) ═O,
(f) OR^{19a},
(g) S(O)_{q}R^{19b},
(h) S(O)₂N(R^{19c})(R^{19d}),
(i) N(R^{19e})S(O)₂R^{19f},
(j) N(R^{19g})(R^{19h}),
(k) B⁷—C(O)—B⁸—R¹⁹ⁱ,
(l) phenyl (which latter group is optionally substituted by halo),
(m) Het^{c} and
(n) Si(R^{20a})(R^{20b})(R^{20c});
R^{19a} to R¹⁹ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, phenyl (which latter group is optionally substituted by halo) and Het^{d}),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het^{e}),
(d) phenyl (which latter group is optionally substituted by halo) or
(e) Het^{f},
provided that R^{19b} does not represent H when q is 1 or 2;
Het^{a} to Het^{f} independently represent 5- or 6-membered heterocyclic groups containing one to four heteroatoms selected from oxygen, nitrogen and/or sulfur, which heterocyclic groups may be substituted by one or more substituents selected from halo, ═O and C₁₋₆ alkyl;
R^{18a}, R^{18b}, R^{18c}, R^{20a}, R^{20b} and R^{20c} independently represent C₁₋₆ alkyl or phenyl (which latter group is optionally substituted by halo or C₁₋₄ alkyl;
unless otherwise specified
(i) alkyl, alkenyl, alkynyl, cycloalkyl, cycloalkenyl, alkylene and alkenylene groups, as well as the alkyl part of alkoxy groups, may be substituted by one or more halo atoms, and
(ii) cycloalkyl and cycloalkenyl groups may comprise one or two rings and may additionally be ring-fused to one or two phenyl groups;
or a protected derivative thereof.

16. A compound of formula XII, 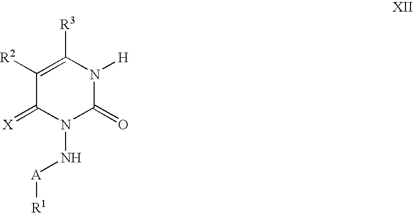
wherein
X represents O or S;
A represents C(O), S(O)₂, C(O)O (in which latter group the O moiety is attached to R¹), C(O)NH, S(O)NH (in which latter two groups the NH moiety is attached to R¹), a direct bond or C₁₋₆ alkylene (which latter group is optionally substituted, at the C-atom to which the NH moiety is attached, by C(O)OR^{A} or C(O)N(H)R^{A});
R^{A} represents H or C₁₋₄ alkyl;
R¹ represents
(a) C₁₋₁₀ alkyl, C₂₋₁₀ alkenyl, C₂₋₁₀ alkynyl (which latter three groups are optionally substituted by one or more substituents selected from halo, CN, C₃₋₁₀ cycloalkyl (optionally substituted by one or more substituents selected from halo, OH, ═O, C₁₋₆ alkyl, C₁₋₆ alkoxy and aryl), OR^{6a}, S(O)ₙR^{6b}, S(O)₂N(R^{6c})(R^{6d}), N(R^{6e})S(O)₂R^{6f}, N(R^{6g})(R^{6h}), B¹—C(O)—B²—R⁶ⁱ, aryl and Het¹),
(b)C₃₋₁₀ cycloalkyl or C₄₋₁₀ cycloalkenyl, which latter two groups are optionally substituted by one or more substituents selected from halo, ═O, CN, C₁₋₁₀ alkyl, C₃₋₁₀ cycloalkyl (optionally substituted by one or more substituents selected from halo, OH, ═O, C₁₋₆ alkyl, C₁₋₆ alkoxy and aryl), OR^{6a}, S(O)ₙR^{6b}, S(O)N(R^{6c})(R^{6d}), N(R^{6e})S(O)₂R^{6f}, N(R^{6g})(R^{6h}), B³—C(O)—B⁴—R⁶ⁱ, aryl and Het²,
(c) aryl, or
(d) Het³;
R^{6a} to R⁶ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, aryl and Het⁴),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, aryl and Het⁵),
(d) aryl or
(e) Het⁶,
provided that R^{6b} does not represent H when n is 1 or 2;
R² represents H or halo;
R³ represents
(a) H,
(b) halo,
(c) CN,
(d) C₁₋₆ alkyl, C₂₋₆ alkenyl, C₂₋₆ alkynyl C₁₋₆ alkoxy (which latter four groups are optionally substituted by one or more substituents selected from halo, OH, CN, C₁₋₄ alkoxy, C(O)OH, C(O)O—C₁₋₄ alkyl and OC(O)—C₁₋₄ alky) or
(e) together with R⁴, R³ represents C₂₋₃ n-alkylene, T¹-(C₁₋₂ n-alkylene) or (C₁₋₂ n-alkylene)-T¹, which latter three groups are optionally substituted by halo, or
(f) together with R⁴ and R⁵, R³ represents T²-[C(H)═], wherein T² is bonded to the C-atom to which the group R³ is attached;
T¹ and T² independently represent O, S, or NR⁷;
R⁷ represents H or C₁₋₄ alkyl;
n, p, and q, independently, represent 0, 1 or 2;
each aryl independently represents a C₆₋₁₀ carbocyclic aromatic group, which group may comprise either one or two rings and may be substituted by one or more substituents selected from
(a) halo,
(b) CN,
(c) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, C(O)OH, C(O)O—C₁₋₆ alkyl, phenyl (which latter group is optionally substituted by halo) and Het⁷),
(d) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het⁸),
(e) OR^{17a},
(f) S(O)ₚR^{17b},
(g) S(O)₂N(R^{17c})(R^{17d}),
(h) N(R^{17e})S(O)₂R^{17f},
(i) N(R^{17g})(R^{17h}),
(j) B⁵—C(O)—B⁶—R¹⁷ⁱ,
(k) phenyl (which latter group is optionally substituted by halo),
(l) Het⁹ and
(m) Si(R^{18a})(R^{18b})(R^{18c}),
R^{17a} to R¹⁷ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, phenyl (which latter group is optionally substituted by halo) and Het¹⁰),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het¹¹),
(d) phenyl (which latter group is optionally substituted by halo) or
(e) Het¹²,
provided that R^{17b} does not represent H when p is 1 or 2;
B¹ to B⁸ independently represent a direct bond, O, S, NH or N—C₁₋₄ alkyl;
Het¹ to Het¹² independently represent 4- to 14-membered heterocyclic groups containing one or more heteroatoms selected from oxygen, nitrogen and/or sulfur, which heterocyclic groups may comprise one, two or three rings and may be substituted by one or more substituents selected from
(a) halo,
(b) CN,
(c) C₁₋₁₀ alkyl, C₂₋₁₀ alkenyl, C₂₋₁₀ alkynyl (which latter four groups are optionally substituted by one or more substituents selected from halo, OH, C₁₋₆ alkoxy, C(O)OH, C(O)O—C₁₋₆ alkyl, phenyl (which latter group is optionally substituted by halo) and Het^{a}),
(d) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het^{b}),
(e) ═O,
(f) OR^{19a},
(g) S(O)_{q}R^{19b},
(h) S(O)₂N(R^{19c})(R^{19d}),
(i) N(R^{19e})S(O)₂R^{19f},
(j) N(R^{19g})(R^{19h}),
(k) B⁷—C(O)—B⁸—R¹⁹ⁱ,
(l) phenyl (which latter group is optionally substituted by halo),
(m) Het^{c} and
(n) Si(R^{20a})(R^{20b})(R^{20c});
R^{19a} to R¹⁹ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, phenyl (which latter group is optionally substituted by halo) and Het^{d}),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, phenyl (which latter group is optionally substituted by halo) and Het^{e}),
(d) phenyl (which latter group is optionally substituted by halo) or
(e) Het^{f},
provided that R^{19b} does not represent H when q is 1 or 2;
Het^{a} to Het^{f} independently represent 5- or 6-membered heterocyclic groups containing one to four heteroatoms selected from oxygen, nitrogen and/or sulfur, which heterocyclic groups may be substituted by one or more substituents selected from halo, ═O and C₁₋₆ alkyl;
R^{18a}, R^{18b}, R^{18c}, R^{20a}, R^{20b} and R^{20c} independently represent C₁₋₆ alkyl or phenyl (which latter group is optionally substituted by halo or C₁₋₄ alkyl);
unless otherwise specified
(i) alkyl, alkenyl, alkynyl, cycloalkyl, cycloalkenyl, alkylene and alkenylene groups, as well as the alkyl part of alkoxy groups, may be substituted by one or more halo atoms, and
(ii) cycloalkyl and cycloalkenyl groups may comprise one or two rings and may additionally be ring-fused to one or two phenyl groups;
or a protected derivative thereof.

17. A compound as claimed in claim 2 wherein X represents S and R³ represents CN or C₁₋₄ alkyl substituted by one or more fluoro atoms.
